SEQUENCE LISTING (32 sequences)

SEQ ID NO: 1 - DNA, 429 bp, synthetic construct
  misc_feature 1..429: note = 3'UTR-WT
  source 1..429: mol_type = other DNA
gcaccaatcttagtgttgtcaggcctgctagtcagccacagcttggggaaagctgtgcagcctgtgacccccccaggagaagctgggaaaccaagcccatagtcaggccgagaacgccatggcacggaagaagccatgctgcctgtgagcccctcagaggacactgagtcaaaaaaccccacgcgcttggaggcgcaggatgggaaaagaaggtggcgaccttccccaccctttaatctggggcctgaactggagatcagctgtggatctccagaagagggactagtggttagaggagaccccccggaaaacgcaaaacagcatattgacgctgggaaagaccagagactccatgagtttccaccacgctggccgccaggcacagatcgccgaatagcggcggccggtgtggggaaatccatggtttct

SEQ ID NO: 2 - DNA, 419 bp, synthetic construct
  misc_feature 1..419: note = 3'UTR-10-del
  source 1..419: mol_type = other DNA
gcaccaatcttagtgttgtcaggcctgctagtcagccacagcttggggaaagctgtgcagcctgtgacccccccaggagaagctgggaaaccaagcccatagtcaggccgagaacgccatggcacggaagaagccatgctgcctgtgagcccctcagaggacactgagtcaaaaaaccccacgcgcttggaggcgcaggatgggaaaagaaggtggcgaccttccccaccctttaatctggggcctgaactggagatcagctgtggatctgactagtggttagaggagaccccccggaaaacgcaaaacagcatattgacgctgggaaagaccagagactccatgagtttccaccacgctggccgccaggcacagatcgccgaatagcggcggccggtgtggggaaatccatggtttct

SEQ ID NO: 3 - DNA, 409 bp, synthetic construct
  misc_feature 1..409: note = 3'UTR-20-del
  source 1..409: mol_type = other DNA
gcaccaatcttagtgttgtcaggcctgctagtcagccacagcttggggaaagctgtgcagcctgtgacccccccaggagaagctgggaaaccaagcccatagtcaggccgagaacgccatggcacggaagaagccatgctgcctgtgagcccctcagaggacactgagtcaaaaaaccccacgcgcttggaggcgcaggatgggaaaagaaggtggcgaccttccccaccctttaatctggggcctgaactggagatcaggactagtggttagaggagaccccccggaaaacgcaaaacagcatattgacgctgggaaagaccagagactccatgagtttccaccacgctggccgccaggcacagatcgccgaatagcggcggccggtgtggggaaatccatggtttct

SEQ ID NO: 4 - DNA, 399 bp, synthetic construct
  misc_feature 1..399: note = 3'UTR-30-del-a
  source 1..399: mol_type = other DNA
gcaccaatcttagtgttgtcaggcctgctagtcagccacagcttggggaaagctgtgcagcctgtgacccccccaggagaagctgggaaaccaagcccatagtcaggccgagaacgccatggcacggaagaagccatgctgcctgtgagcccctcagaggacactgagtcaaaaaaccccacgcgcttggaggcgcaggatgggaaaagaaggtggcgaccttccccaccctttaatctggggcctgaacgactagtggttagaggagaccccccggaaaacgcaaaacagcatattgacgctgggaaagaccagagactccatgagtttccaccacgctggccgccaggcacagatcgccgaatagcggcggccggtgtggggaaatccatggtttct

SEQ ID NO: 5 - DNA, 399 bp, synthetic construct
  misc_feature 1..399: note = 3'UTR-30-del-b
  source 1..399: mol_type = other DNA
gcaccaatcttagtgttgtcaggcctgctagtcagccacagcttggggaaagctgtgcagcctgtgacccccccaggagaagctgggaaaccaagcccatagtcaggccgagaacgccatggcacggaagaagccatgctgcctgtgagcccctcagaggacactgagtcaaaaaaccccacgcgcttggaggcgcaggatgggaaaagaaggtggcgaccttccccaccctttaatctggggcctgaactggagatcagctgtggagaccccccggaaaacgcaaaacagcatattgacgctgggaaagaccagagactccatgagtttccaccacgctggccgccaggcacagatcgccgaatagcggcggccggtgtggggaaatccatggtttct

SEQ ID NO: 6 - DNA, 18611 bp, synthetic construct
  misc_feature 1..18611: note = CMV ZIKV FL sequence
  source 1..18611: mol_type = other DNA
agttgttgatctgtgtgaatcagactgcgacagttcgagtttgaagcgaaagctagcaacagtatcaacaggttttattttggatttggaaacgagagtttctggtcatgaaaaacccaaagaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagtggggaaaaaagaggctatggaaataataaagaagtttaagaaagatctggctgccatgctgagaataatcaatgctaggaaggagaagaagagacgaggcacagatactagtgtcggaattgttggcctcctgctgaccacagccatggcagtggaggtcactagacgtgggaatgcatactatatgtacttggacagaagcgatgctggggaggccatatcttttccaaccacaatggggatgaataagtgttatatacagatcatggatcttggacacatgtgtgatgccaccatgagctatgaatgccctatgctggatgagggggtagaaccagatgacgtcgattgttggtgcaacacgacgtcaacttgggttgtgtacggaacctgccaccacaaaaaaggtgaagcacggagatctagaagagctgtgacgctcccctcccattccactaggaagctgcaaacgcggtcgcagacctggttggaatcaagagaatacacaaagcacctgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgttaccgtaatggcacaggacaaaccgactgtcgacatagagctggttacaacaacagtcagcaacatggcggaggtaagatcctactgctatgaggcatcaatatcggacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcttgctctaagaaaatgaccgggaagagcatccagccagagaatctggagtaccggataatgctgtcagttcatggctcccagcacagtgggatgatcgttaatgatacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcagactgtcgtggttctagggagtcaagaaggagcagttcacacggcccttgctggagctctggaggctgagatggatggtgcaaagggaaggctgtcctctggccacttgaaatgtcgcctgaaaatggacaaacttagattgaagggcgtgtcatactccttgtgtaccgcagcgttcacattcactaagatcccggctgaaacactgcacgggacagtcacagtggaggtacagtacgcagggacagatggaccttgcaaggttccagctcagatggcggtggacatgcaaactctgaccccagttgggaggttgataaccgctaaccctgtaatcactgaaagcactgagaactccaagatgatgctggaactggatccaccatttggggactcttacattgtcataggagtcggggaaaagaagatcacccaccactggcacaggagtggcagcaccattggaaaagcatttgaagccactgtgagaggtgccaagagaatggcagtcttgggagacacagcctgggactttggatcagttgggggtgctctcaactcactgggcaagggcatccatcaaatttttggagcagctttcaaatcattgtttggaggaatgtcctggttctcacaaattctcattggaacgttgctggtgtggttgggtctgaatacaaagaatggatctatttcccttatgtgcttggccttagggggagtgttgatcttcttatccacagccgtctctgctgatgtggggtgctcggtggacttctcaaagaaggaaacgagatgcggtacaggggtgttcgtctataacgacgttgaagcttggagggacaggtacaagtaccatcctgactcccctcgtagattggcagcagcagtcaagcaagcctgggaagatgggatctgtgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccatggctggaaggcttgggggaaatcgtacttcgtcagggcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcactcgagtgtgatccagccgtcattggaacagccgctaagggaaaggaggctgtgcacagtgatctaggctactggattgagagtgagaagaacgacacatggaggctgaagagggcccacctgatcgagatgaaaacatgtgaatggccaaagtcccacacattgtggacagatggaatagaagaaagtgatctgatcatacccaagtctttagctgggccactcagccatcacaacaccagagagggctacaggacccaaatgaaagggccatggcatagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggttgttggtatggaatggagataaggcccaggaaagaaccagaaagtaacttagtaaggtcaatggtgactgcaggatcaactgatcacatggatcacttctcccttggagtgcttgtgattctgctcatggtacaggaagggctaaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgttgctcatctggcgctgatagcggcattcaaagtcagacctgcgttgctggtatctttcattttcagagctaattggacaccccgtgagagcatgctgctggccttggcctcgtgtcttctgcaaactgcgatctccgccttggaaggcgacctgatggttcccatcaatggttttgctttggcctggttggcaatacgagcgatggttgttccacgcactgacaacatcaccttggcaatcctggctgctctgacaccactggcccggggcacactgcttgtggcgtggagagcaggccttgctacttgcggggggttcatgctcctttctctgaaggggaaaggcagtgtgaagaagaacttaccatttgtcatggccctgggactaaccgctgtgaggctggtcgaccccatcaacgtggtgggactgctgttgctcacaaggagtgggaagcggagctggccccctagtgaagtactcacagctgttggcctgatatgcgcattggctggagggttcgccaaggcggatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcactagatgagagtggtgatttctccctagtggaggatgatggtccccccatgagagagatcatactcaaagtggtcctgatggccatctgtggcatgaacccaatagccataccctttgcagctggagcgtggtacgtgtatgtgaagactggaaaaaggagtggtgctctatgggatgtgcctgctcccaaggaagtaaaaaagggggagaccacagatggagtgtacagagtaatgactcgtagactgctaggttcaacacaagttggagtgggagtcatgcaagagggggtcttccacactatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcagttgcgctggactacccagcaggtacctcaggatctccaatcctagataagtgtgggagagtgataggactctatggtaatggggtcgtgatcaaaaatgggagttacgttagtgccatcacccaagggaggagggaggaagagactcctgttgagtgcttcgagccttcgatgctgaagaagaagcagctaactgtcttagacttgcatcctggagctgggaaaaccaggagagttcttcctgaaatagtccgtgaagccataaaaacaagactccgcactgtgatcttagctccaaccagggttgtcgctgctgaaatggaggaagcccttagagggcttccagtgcgttatatgacaacagcagtcaatgtcacccattctgggacagaaatcgttgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgactgccacgccaccaggaacccgtgacgcattcccggactccaactcaccaattatggacaccgaagtggaagtcccagagagagcctggagctcaggctttgattgggtgacggatcattctggaaaaacagtttggtttgttccaagcgtgaggaatggcaatgagatcgcagcttgtctgacaaaggctggaaaacgggtcatacagctcagcagaaagacttttgagacagagttccagaaaacaaaacatcaagagtgggacttcgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaaccccaacaaacctggagatgagtatctgtatggaggtgggtgcgcagagactgatgaagaccatgcacactggcttgaagcaagaatgcttcttgacaacatttacctccaagatggcctcatagcctcgctctatcgacctgaggccgacaaagtagcagctattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagatacggagagaaaagagtgctcaaaccgaggtggatggacgccagagtttgttcagatcatgcggccctgaagtcattcaaagagtttgccgctgggaaaagaggagcggcctttggagtgatggaagccctgggaacactgccaggacatatgacagagagattccaggaggccattgacaacctcgctgtgctcatgcgggcagagactggaagcaggccctacaaagccgcggcggcccaattaccggagaccctagagactatcatgcttttggggttgctgggaacagtctcgctgggaatctttttcgtcttgatgcggaacaagggcatagggaagatgggctttggaatggtgactcttggggccagcgcatggcttatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtgggtcttctgggcttgattaccgccaatgaactcggatggttggagagaacaaagagtgacctaagccatctaatgggaaggagagaggagggggcaactataggattctcaatggacattgacctgcggccagcctcagcttgggctatctatgctgctctgacaactttcattaccccagccgtccaacatgcagtgaccacttcatacaacaactactccttaatggcgatggccacgcaagctggagtgttgttcggtatgggtaaagggatgccattctatgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagctgtctccagcgccatactgtcgcggaccgcctgggggtggggtgaggctggggccctgatcacagctgcaacttccactttgtgggagggctctccgaacaagtactggaactcctccacagccacctcactgtgtaacatttttaggggaagctacttggctggagcttctctaatctacacagtaacaagaaacgctggcttggtcaagagacgtgggggtggaacgggagagaccctgggagagaaatggaaggcccgcctgaaccagatgtcggccctggagttctactcctacaaaaagtcaggcatcaccgaggtgtgcagagaagaggcccgccgcgccctcaaggacggtgtggcaacgggaggccacgctgtgtcccgaggaagtgcaaagctgagatggttggtggagaggggatacctgcagccctatggaaaggtcattgatcttggatgtggcagagggggctggagttactatgccgccaccatccgcaaagttcaagaagtgaaaggatacacaaaaggaggccctggtcatgaagaacccatgttggtgcaaagctatgggtggaacatagtccgtcttaagagtggggtggacgtctttcatatggcggctgagccgtgtgacacgttgctgtgtgatataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctccttttggggcgcatggacgggcccaggaggccagtgaaatatgaagaggatgtgaatctcggctctggcacgcgggctgtggtaagctgcgctgaagctcccaacatgaagatcattggtaaccgcattgagaggatccgcagtgagcacgcggaaacgtggttctttgacgagaaccacccatataggacatgggcttaccatggaagctacgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagttaggcaaacacaaacggccacgagtctgtaccaaagaagagttcatcaacaaggttcgtagcaacgcagcattaggggcaatatttgaagaggaaaaagagtggaagactgcagtggaagctgtgaacgatccaaggttctgggctctagtggacaaggaaagagagcaccacctgagaggagagtgccagagctgtgtgtacaacatgatgggaaaaagagaaaagaaacaaggggaatttggaaaggccaagggcagccgcgccatctggtacatgtggctaggggctagatttctagagttcgaagcccttggattcttgaacgaggatcactggatggggagagagaattcaggaggtggtgttgaagggctaggattacaaagactcggatatgtcttagaagagatgagtcgcataccaggaggaaggatgtatgcagatgatactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaagacagttatggacattatttcaagacaagaccaaagggggagcggacaagttgtcacttacgctcttaatacatttaccaacctagtggtgcagctcattcggaatatggaggctgaggaagttctagagatgcaagacttgtggctgctgcggaggtcagagaaagtgaccaactggttgcagagcaatggatgggataggctcaaacgaatggcagtcagtggagatgattgcgttgtgaaaccaattgatgataggtttgcacatgctctcaggttcttgaatgatatgggaaaagttaggaaggacacacaagagtggaagccctcaactggatgggacaactgggaagaagttccgttttgctcccaccacttcaacaagctccatctcaaggacgggaggtccattgtggttccctgccgccaccaagatgaactgattggccgagctcgcgtctcaccgggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtgggtctctcatagggcacagaccgcgcaccacctgggctgagaacattaaaaacacagtcaacatgatgcgtaggatcataggtgatgaagaaaagtacgtggactacctatccacccaagttcgctacttgggcgaagaagggtccacacctggagtgctataagcaccaatcttagtgttgtcaggcctgctagtcagccacagcttggggaaagctgtgcagcctgtgacccccccaggagaagctgggaaaccaagcccatagtcaggccgagaacgccatggcacggaagaagccatgctgcctgtgagcccctcagaggacactgagtcaaaaaaccccacgcgcttggaggcgcaggatgggaaaagaaggtggcgaccttccccaccctttaatctggggcctgaactggagatcagctgtggatctccagaagagggactagtggttagaggagaccccccggaaaacgcaaaacagcatattgacgctgggaaagaccagagactccatgagtttccaccacgctggccgccaggcacagatcgccgaatagcggcggccggtgtggggaaatccatgggtctgggtcggcatggcatctccacctcctcgcggtccgacctgggctacttcggtaggctaagggagaagaatcgatgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaac

SEQ ID NO: 7 - DNA, 18258 bp, synthetic construct
  misc_feature 1..18258: note = SV40 ZIKV FL sequence
  source 1..18258: mol_type = other DNA
agttgttgatctgtgtgaatcagactgcgacagttcgagtttgaagcgaaagctagcaacagtatcaacaggttttattttggatttggaaacgagagtttctggtcatgaaaaacccaaagaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagtggggaaaaaagaggctatggaaataataaagaagtttaagaaagatctggctgccatgctgagaataatcaatgctaggaaggagaagaagagacgaggcacagatactagtgtcggaattgttggcctcctgctgaccacagccatggcagtggaggtcactagacgtgggaatgcatactatatgtacttggacagaagcgatgctggggaggccatatcttttccaaccacaatggggatgaataagtgttatatacagatcatggatcttggacacatgtgtgatgccaccatgagctatgaatgccctatgctggatgagggggtagaaccagatgacgtcgattgttggtgcaacacgacgtcaacttgggttgtgtacggaacctgccaccacaaaaaaggtgaagcacggagatctagaagagctgtgacgctcccctcccattccactaggaagctgcaaacgcggtcgcagacctggttggaatcaagagaatacacaaagcacctgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgttaccgtaatggcacaggacaaaccgactgtcgacatagagctggttacaacaacagtcagcaacatggcggaggtaagatcctactgctatgaggcatcaatatcggacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcttgctctaagaaaatgaccgggaagagcatccagccagagaatctggagtaccggataatgctgtcagttcatggctcccagcacagtgggatgatcgttaatgatacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcagactgtcgtggttctagggagtcaagaaggagcagttcacacggcccttgctggagctctggaggctgagatggatggtgcaaagggaaggctgtcctctggccacttgaaatgtcgcctgaaaatggacaaacttagattgaagggcgtgtcatactccttgtgtaccgcagcgttcacattcactaagatcccggctgaaacactgcacgggacagtcacagtggaggtacagtacgcagggacagatggaccttgcaaggttccagctcagatggcggtggacatgcaaactctgaccccagttgggaggttgataaccgctaaccctgtaatcactgaaagcactgagaactccaagatgatgctggaactggatccaccatttggggactcttacattgtcataggagtcggggaaaagaagatcacccaccactggcacaggagtggcagcaccattggaaaagcatttgaagccactgtgagaggtgccaagagaatggcagtcttgggagacacagcctgggactttggatcagttgggggtgctctcaactcactgggcaagggcatccatcaaatttttggagcagctttcaaatcattgtttggaggaatgtcctggttctcacaaattctcattggaacgttgctggtgtggttgggtctgaatacaaagaatggatctatttcccttatgtgcttggccttagggggagtgttgatcttcttatccacagccgtctctgctgatgtggggtgctcggtggacttctcaaagaaggaaacgagatgcggtacaggggtgttcgtctataacgacgttgaagcttggagggacaggtacaagtaccatcctgactcccctcgtagattggcagcagcagtcaagcaagcctgggaagatgggatctgtgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccatggctggaaggcttgggggaaatcgtacttcgtcagggcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcactcgagtgtgatccagccgtcattggaacagccgctaagggaaaggaggctgtgcacagtgatctaggctactggattgagagtgagaagaacgacacatggaggctgaagagggcccacctgatcgagatgaaaacatgtgaatggccaaagtcccacacattgtggacagatggaatagaagaaagtgatctgatcatacccaagtctttagctgggccactcagccatcacaacaccagagagggctacaggacccaaatgaaagggccatggcatagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggttgttggtatggaatggagataaggcccaggaaagaaccagaaagtaacttagtaaggtcaatggtgactgcaggatcaactgatcacatggatcacttctcccttggagtgcttgtgattctgctcatggtacaggaagggctaaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgttgctcatctggcgctgatagcggcattcaaagtcagacctgcgttgctggtatctttcattttcagagctaattggacaccccgtgagagcatgctgctggccttggcctcgtgtcttctgcaaactgcgatctccgccttggaaggcgacctgatggttcccatcaatggttttgctttggcctggttggcaatacgagcgatggttgttccacgcactgacaacatcaccttggcaatcctggctgctctgacaccactggcccggggcacactgcttgtggcgtggagagcaggccttgctacttgcggggggttcatgctcctttctctgaaggggaaaggcagtgtgaagaagaacttaccatttgtcatggccctgggactaaccgctgtgaggctggtcgaccccatcaacgtggtgggactgctgttgctcacaaggagtgggaagcggagctggccccctagtgaagtactcacagctgttggcctgatatgcgcattggctggagggttcgccaaggcggatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcactagatgagagtggtgatttctccctagtggaggatgatggtccccccatgagagagatcatactcaaagtggtcctgatggccatctgtggcatgaacccaatagccataccctttgcagctggagcgtggtacgtgtatgtgaagactggaaaaaggagtggtgctctatgggatgtgcctgctcccaaggaagtaaaaaagggggagaccacagatggagtgtacagagtaatgactcgtagactgctaggttcaacacaagttggagtgggagtcatgcaagagggggtcttccacactatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcagttgcgctggactacccagcaggtacctcaggatctccaatcctagataagtgtgggagagtgataggactctatggtaatggggtcgtgatcaaaaatgggagttacgttagtgccatcacccaagggaggagggaggaagagactcctgttgagtgcttcgagccttcgatgctgaagaagaagcagctaactgtcttagacttgcatcctggagctgggaaaaccaggagagttcttcctgaaatagtccgtgaagccataaaaacaagactccgcactgtgatcttagctccaaccagggttgtcgctgctgaaatggaggaagcccttagagggcttccagtgcgttatatgacaacagcagtcaatgtcacccattctgggacagaaatcgttgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgactgccacgccaccaggaacccgtgacgcattcccggactccaactcaccaattatggacaccgaagtggaagtcccagagagagcctggagctcaggctttgattgggtgacggatcattctggaaaaacagtttggtttgttccaagcgtgaggaatggcaatgagatcgcagcttgtctgacaaaggctggaaaacgggtcatacagctcagcagaaagacttttgagacagagttccagaaaacaaaacatcaagagtgggacttcgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaaccccaacaaacctggagatgagtatctgtatggaggtgggtgcgcagagactgatgaagaccatgcacactggcttgaagcaagaatgcttcttgacaacatttacctccaagatggcctcatagcctcgctctatcgacctgaggccgacaaagtagcagctattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagatacggagagaaaagagtgctcaaaccgaggtggatggacgccagagtttgttcagatcatgcggccctgaagtcattcaaagagtttgccgctgggaaaagaggagcggcctttggagtgatggaagccctgggaacactgccaggacatatgacagagagattccaggaggccattgacaacctcgctgtgctcatgcgggcagagactggaagcaggccctacaaagccgcggcggcccaattaccggagaccctagagactatcatgcttttggggttgctgggaacagtctcgctgggaatctttttcgtcttgatgcggaacaagggcatagggaagatgggctttggaatggtgactcttggggccagcgcatggcttatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtgggtcttctgggcttgattaccgccaatgaactcggatggttggagagaacaaagagtgacctaagccatctaatgggaaggagagaggagggggcaactataggattctcaatggacattgacctgcggccagcctcagcttgggctatctatgctgctctgacaactttcattaccccagccgtccaacatgcagtgaccacttcatacaacaactactccttaatggcgatggccacgcaagctggagtgttgttcggtatgggtaaagggatgccattctatgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagctgtctccagcgccatactgtcgcggaccgcctgggggtggggtgaggctggggccctgatcacagctgcaacttccactttgtgggagggctctccgaacaagtactggaactcctccacagccacctcactgtgtaacatttttaggggaagctacttggctggagcttctctaatctacacagtaacaagaaacgctggcttggtcaagagacgtgggggtggaacgggagagaccctgggagagaaatggaaggcccgcctgaaccagatgtcggccctggagttctactcctacaaaaagtcaggcatcaccgaggtgtgcagagaagaggcccgccgcgccctcaaggacggtgtggcaacgggaggccacgctgtgtcccgaggaagtgcaaagctgagatggttggtggagaggggatacctgcagccctatggaaaggtcattgatcttggatgtggcagagggggctggagttactatgccgccaccatccgcaaagttcaagaagtgaaaggatacacaaaaggaggccctggtcatgaagaacccatgttggtgcaaagctatgggtggaacatagtccgtcttaagagtggggtggacgtctttcatatggcggctgagccgtgtgacacgttgctgtgtgatataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctccttttggggcgcatggacgggcccaggaggccagtgaaatatgaagaggatgtgaatctcggctctggcacgcgggctgtggtaagctgcgctgaagctcccaacatgaagatcattggtaaccgcattgagaggatccgcagtgagcacgcggaaacgtggttctttgacgagaaccacccatataggacatgggcttaccatggaagctacgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagttaggcaaacacaaacggccacgagtctgtaccaaagaagagttcatcaacaaggttcgtagcaacgcagcattaggggcaatatttgaagaggaaaaagagtggaagactgcagtggaagctgtgaacgatccaaggttctgggctctagtggacaaggaaagagagcaccacctgagaggagagtgccagagctgtgtgtacaacatgatgggaaaaagagaaaagaaacaaggggaatttggaaaggccaagggcagccgcgccatctggtacatgtggctaggggctagatttctagagttcgaagcccttggattcttgaacgaggatcactggatggggagagagaattcaggaggtggtgttgaagggctaggattacaaagactcggatatgtcttagaagagatgagtcgcataccaggaggaaggatgtatgcagatgatactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaagacagttatggacattatttcaagacaagaccaaagggggagcggacaagttgtcacttacgctcttaatacatttaccaacctagtggtgcagctcattcggaatatggaggctgaggaagttctagagatgcaagacttgtggctgctgcggaggtcagagaaagtgaccaactggttgcagagcaatggatgggataggctcaaacgaatggcagtcagtggagatgattgcgttgtgaaaccaattgatgataggtttgcacatgctctcaggttcttgaatgatatgggaaaagttaggaaggacacacaagagtggaagccctcaactggatgggacaactgggaagaagttccgttttgctcccaccacttcaacaagctccatctcaaggacgggaggtccattgtggttccctgccgccaccaagatgaactgattggccgagctcgcgtctcaccgggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtgggtctctcatagggcacagaccgcgcaccacctgggctgagaacattaaaaacacagtcaacatgatgcgtaggatcataggtgatgaagaaaagtacgtggactacctatccacccaagttcgctacttgggcgaagaagggtccacacctggagtgctataagcaccaatcttagtgttgtcaggcctgctagtcagccacagcttggggaaagctgtgcagcctgtgacccccccaggagaagctgggaaaccaagcccatagtcaggccgagaacgccatggcacggaagaagccatgctgcctgtgagcccctcagaggacactgagtcaaaaaaccccacgcgcttggaggcgcaggatgggaaaagaaggtggcgaccttccccaccctttaatctggggcctgaactggagatcagctgtggatctccagaagagggactagtggttagaggagaccccccggaaaacgcaaaacagcatattgacgctgggaaagaccagagactccatgagtttccaccacgctggccgccaggcacagatcgccgaatagcggcggccggtgtggggaaatccatgggtctgggtcggcatggcatctccacctcctcgcggtccgacctgggctacttcggtaggctaagggagaagaatcgatgaacttgtttattgcagcttataatggttacaaataaagcaatagcatcacaaatttcacaaataaagcatttttttcactgcattctagttgtggtttgtccaaactcatcaatgtatcttatgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaactgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctc

SEQ ID NO: 8 - DNA, 10 bp, synthetic construct
  misc_feature 1..10: note = 3' UTR Zika 10 Nucleotide Deletion
  source 1..10: mol_type = other DNA
ccagaagagg

SEQ ID NO: 9 - DNA, 20 bp, synthetic construct
  misc_feature 1..20: note = 3' UTR Zika 20 Nucleotide Deletion
  source 1..20: mol_type = other DNA
ctgtggatctccagaagagg

SEQ ID NO: 10 - DNA, 17 bp, synthetic construct
  misc_feature 1..17: note = forward primer - 1193F
  source 1..17: mol_type = other DNA
ccgctgcccaacacaag

SEQ ID NO: 11 - DNA, 24 bp, synthetic construct
  misc_feature 1..24: note = reverse primer - 1269R
  source 1..24: mol_type = other DNA
ccactaacgttcttttgcagacat

SEQ ID NO: 12 - DNA, 31 bp, synthetic construct
  misc_feature 1..31: note = probe
  misc_feature 1: note = MOD_RES - linked to FAM
  misc_feature 10: note = MOD_RES - linked to Zen
  source 1..31: mol_type = other DNA
agcctaccttgacaagcaatcagacactcaa

SEQ ID NO: 13 - DNA, 28 bp, synthetic construct
  misc_feature 1..28: note = forward primer - 1183F
  source 1..28: mol_type = other DNA
ccaccaatgttctcttgcagacatattg

SEQ ID NO: 14 - DNA, 26 bp, synthetic construct
  misc_feature 1..26: note = reverse primer - 1268R
  source 1..26: mol_type = other DNA
ttcggacagccgttgtccaacacaag

SEQ ID NO: 15 - DNA, 21 bp, synthetic construct
  misc_feature 1..21: note = probe
  misc_feature 1: note = MOD_RES - linked to FAM
  misc_feature 10: note = MOD_RES - linked to Zen
  source 1..21: mol_type = other DNA
agcctaccttgacaagcagtc

SEQ ID NO: 16 - DNA, 17 bp, synthetic construct
  misc_feature 1..17: note = forward primer - 1193F
  source 1..17: mol_type = other DNA
ccgctgcccaacacaag

SEQ ID NO: 17 - DNA, 24 bp, synthetic construct
  misc_feature 1..24: note = reverse primer - 1269R
  source 1..24: mol_type = other DNA
ccactaacgttcttttgcagacat

SEQ ID NO: 18 - DNA, 31 bp, synthetic construct; identical to SEQ ID NO: 12
  misc_feature 1..31: note = probe
  misc_feature 1: note = MOD_RES - linked to FAM
  misc_feature 10: note = MOD_RES - linked to Zen
  source 1..31: mol_type = other DNA

SEQ ID NO: 19 - DNA, 22 bp, synthetic construct
  misc_feature 1..22: note = probe - 3IABkFQ-3'
  source 1..22: mol_type = other DNA
tgacaagcaatcagacactcaa

SEQ ID NO: 20 - DNA, 45 bp, synthetic construct
  misc_feature 1..45: note = WT
  source 1..45: mol_type = other DNA
tggagatcagctgtggatctccagaagagggactagtggttagag

SEQ ID NO: 21 - DNA, 34 bp, synthetic construct
  misc_feature 1..34: note = 10-del
  source 1..34: mol_type = other DNA
tggagatcagctgtggatcgactagtggttagag

SEQ ID NO: 22 - DNA, 25 bp, synthetic construct
  misc_feature 1..25: note = 20-del
  source 1..25: mol_type = other DNA
tggagatcaggactagtggttagag

SEQ ID NO: 23 - DNA, 15 bp, synthetic construct
  misc_feature 1..15: note = 30-del-a
  source 1..15: mol_type = other DNA
gactagtggttagag

SEQ ID NO: 24 - DNA, 15 bp, synthetic construct
  misc_feature 1..15: note = 30-del-b
  source 1..15: mol_type = other DNA
tggagatcagctgtg

SEQ ID NO: 25 - DNA, 45 bp, synthetic construct; identical to SEQ ID NO: 20
  misc_feature 1..45: note = FSS13025/KU955593
  source 1..45: mol_type = other DNA

SEQ ID NO: 26 - DNA, 45 bp, synthetic construct; identical to SEQ ID NO: 20
  misc_feature 1..45: note = H/PF2013/KJ776791
  source 1..45: mol_type = other DNA

SEQ ID NO: 27 - DNA, 45 bp, synthetic construct; identical to SEQ ID NO: 20
  misc_feature 1..45: note = PRVABC 59/KU501215
  source 1..45: mol_type = other DNA

SEQ ID NO: 28 - DNA, 45 bp, synthetic construct; identical to SEQ ID NO: 20
  misc_feature 1..45: note = Natal RGN/KU527068
  source 1..45: mol_type = other DNA

SEQ ID NO: 29 - DNA, 45 bp, synthetic construct; identical to SEQ ID NO: 20
  misc_feature 1..45: note = ZKV2015/KU497555
  source 1..45: mol_type = other DNA

SEQ ID NO: 30 - DNA, 45 bp, synthetic construct
  misc_feature 1..45: note = P6-740/KX377336
  source 1..45: mol_type = other DNA
tggagatcagctgtggatctccaggagagggactagcggttagag

SEQ ID NO: 31 - DNA, 45 bp, synthetic construct
  misc_feature 1..45: note = MR 766/ AY632535
  source 1..45: mol_type = other DNA
tggagactagctgtgaatctccagcagagggactagtggttagag

SEQ ID NO: 32 - DNA, 45 bp, synthetic construct
  misc_feature 1..45: note = DAK-41525/KU955591
  source 1..45: mol_type = other DNA
tggagattagctgtgaatctccggcagaaggactagtggttagag

(19) United States
(12) Patent Application Publication
(10) Pub. No.: US 2025/0000963 A1
(43) Pub. Date: Jan. 2, 2025

(54) LIVE ATTENUATED ZIKA VIRUS WITH 3'UTR DELETION, VACCINE CONTAINING AND USE THEREOF

(71) Applicant: Board of Regents, The University of Texas System, Austin, TX (US)

(72) Inventors: Pei-Yong SHI, Galveston, TX (US); Xuping XIE, Galveston, TX (US); Chao SHAN, Galveston, TX (US)

(21) Appl. No.: 18/210,829

(22) Filed: Jun. 16, 2023

Related U.S. Application Data
(63) Continuation of application No. 16/485,818, filed on Aug. 14, 2019, now Pat. No. 11,730,801.
(60) Provisional application No. 62/458,839, filed on Feb. 14, 2017.

(51) Int. Cl. A61K 39/12; A61K 39/00; A61P 31/14

(52) U.S. Cl. CPC A61K 39/12; A61P 31/14; A61K 2039/53; A61K 2039/555; A61K 2039/57

(57) ABSTRACT

The present invention discloses a live attenuated strain of Zika virus (ZIKV) having a deletion in the 3′ untranslated region (3′UTR) of the viral genome, which may affect viral RNA synthesis and sensitivity to type I interferon inhibition, but may not affect viral RNA translation. The present invention also discloses the use of these live attenuated ZIKV strains in the preparation of ZIKV vaccines and for providing immunoprotection against ZIKV infection and congenital ZIKV syndrome, particularly in pregnant females.

### RELATED APPLICATIONS

This Application is a continuation of U.S. application Ser. No. 16/485,818 filed Aug. 14, 2019, which is a 371 application of PCT/US2018/018114 filed Feb. 14, 2018, which claims priority to U.S. Provisional Application No. 62/458,839, filed on Feb. 14, 2017, the contents of each are incorporated by reference in their entirety herein.

### STATEMENT REGARDING FEDERALLY SPONSORED RESEARCH OR DEVELOPMENT

This invention was made with government support under AI120942 awarded by the National Institutes of Health. The government has certain rights in the invention.

### REFERENCE TO SEQUENCE LISTING

A sequence listing required by 37 CFR 1.821-1.825 is being submitted electronically with this application. The sequence listing is incorporated herein by reference. The sequence listing that is contained in the file named “UTMB-P0395USC1” which is 81 KB and was created on Jun. 5, 2024.

### FIELD OF THE INVENTION

The invention generally relates to the development of a live attenuated strain of Zika virus (ZIKV) and vaccine compositions comprising this strain. The strain and vaccines comprising it may be used in humans and animals for treating or providing immunoprotection against ZIKV, which may cause congenital ZIKV syndrome and Guillan-Barré syndrome. The invention specifically discloses methods of protecting against congenital ZIKV syndrome, including microcephaly.

### BACKGROUND OF THE INVENTION

The mosquito-borne ZIKV has recently caused a global threat to public health. The most devastating disease associated with Zika virus (ZIKV) infection is the wide range of congenital abnormalities (including microcephaly) now collectively known as congenital ZIKV syndrome (Reference 1). Prevention of congenital ZIKV syndrome is the most pressing task to reduce the burden of epidemics on family and society (Reference 2). In particular, pregnant women without ZIKV immunity in endemic countries are at risk for fetal infection and congenital defects. Since ZIKV could also be sexually transmitted, women living in non-endemic regions can also be at risk when exposed to men who have traveled to endemic countries.

ZIKV is spread to people primarily through the bite of an infected Aedes species mosquito. The most common symptoms of ZIKV are fever, rash, joint pain, and conjunctivitis. The illness is usually mild, with symptoms appearing 2 to 7 days after being bitten by an infected mosquito and lasting for several days to a week. However, there have been reports of congenital ZIKV syndrome, e.g., serious birth defects, especially microcephaly, and other poor pregnancy outcomes in babies of mothers who were infected with ZIKV while pregnant. There have also been cases of Guillain-Barré syndrome (GBS) reported in patients following suspected ZIKV infection. GBS is a rare disorder where a person's own immune system damages the nerve cells, causing muscle weakness and sometimes, paralysis. These symptoms can last anywhere from a few weeks to several months, although some people have permanent damage and, in rare cases, GBS may result in death.

ZIKV is a member of the flavivirus genus (in the family Flaviviridae), which also includes other important human pathogens, e.g., yellow fever (YFV), West Nile (WNV), Japanese encephalitis (JEV), tick-borne encephalitis (TBEV), and Dengue viruses (DENV). Like other members of the flavivirus genus, Zika contains a positive single-stranded genomic RNA, encoding a polyprotein that is processed into three structural proteins (capsid [C], premembrane [prM], and envelope [E] proteins) and seven nonstructural proteins (NS1, NS2A, NS2B, NS3, NS4A, NS4B, and NS5). Structural proteins form virions, whereas nonstructural proteins participate in viral RNA synthesis, virion assembly, and evasion of immune response.

Both inactivated and live-attenuated vaccines have been developed for flaviviruses, including YFV, JEV, TBEV, and DENV (Reference 3). Rapid and promising progress has been made toward ZIKV vaccine development (References 4 and 5). Inactivated ZIKV and subunit vaccines (expressing viral prM/E proteins) have shown efficacy in mice and nonhuman primates (References 6-8). A successful vaccine requires a fine balance between immunogenicity and safety. Live-attenuated vaccines generally offer fast and durable immunity, but sometimes with the trade-off of reduced safety; whereas inactivated and subunit vaccines provide enhanced safety at the cost of reduced immunogenicity, and often require multiple doses and periodic boosters.

The present invention addresses the need for novel ZIKV vaccines that serve at-risk populations in order to treat and/or provide immunoprotection against infections elicited by ZIKV and to prevent congenital ZIKV syndrome, especially microcephaly.

### BRIEF SUMMARY OF THE INVENTION

The invention in general relates to a live attenuated Zika virus (ZIKV) strain, comprising a deletion in the 3′ untranslated region (3′UTR) of the ZIKV genome.

The invention more specifically relates to a live attenuated Zika virus (ZIKV) strain, wherein the 3′UTR deletion ranges from a 10-nucleotide deletion to a 50-nucleotide deletion (i.e., Δ10, Δ11, Δ12, Δ13, Δ14, Δ15, Δ16, Δ17, Δ18, Δ19, Δ20, Δ21, Δ22, Δ23, Δ24, Δ25, Δ26, Δ27, Δ28, Δ29, Δ30, Δ31, Δ32, Δ33, Δ34, Δ35, Δ36, Δ37, Δ38, Δ39, Δ40, Δ41, Δ42, Δ43, Δ44, Δ45, Δ46, Δ47, Δ48, Δ49 or Δ50 3′UTR deletion), in exemplary embodiments the 3′UTR deletion is a 10-nucleotide deletion, a 20-nucleotide deletion, or a 30-nucleotide deletion.

The invention more specifically relates to a live attenuated Zika virus (ZIKV) strain comprising a 3′UTR having at least 80%, at least 85%, at least 90%, at least 95%, at least 98%, at least 99%, or 100% identity to the nucleic acid sequence of SEQ ID NO: 2, 3, 4, or 5.

The invention also specifically relates to a live attenuated Zika virus (ZIKV) strain as above-described which is incompetent in infecting mosquitoes.

The invention also specifically relates to a live attenuated Zika virus (ZIKV) strain as above-described which exhibits decreased viral RNA synthesis compared to wildtype ZIKV strains.

The invention also specifically relates to a live attenuated Zika virus (ZIKV) strain as above-described which exhibits increased sensitivity to type-I interferon inhibition compared to wildtype ZIKV strains.

The invention also specifically relates to a live attenuated Zika virus (ZIKV) strain as above-described wherein the deletion does not affect viral RNA translation.

The invention also specifically relates to a live attenuated Zika virus (ZIKV) strain as above-described which is an mCherry ZIKV strain.

The invention also specifically relates to a live attenuated Zika virus (ZIKV) strain as above-described which comprises or consists of a deletion variant of SEQ ID NO:6 wherein the sequence “CCAGAAGAGG” (3′UTR 10-nucleotide deletion) (SEQ ID NO:8) is deleted therefrom.

The invention also specifically relates to a live attenuated Zika virus (ZIKV) strain as above-described which comprises or consists of a deletion variant of SEQ ID NO:6 wherein the sequence “CTGTGGATCTCCAGAAGAGG” (3′UTR 20-nucleotide deletion) (SEQ ID NO:9) is deleted therefrom.

The invention also specifically relates to a live attenuated Zika virus (ZIKV) strain as above-described which comprises or consists of a deletion variant of SEQ ID NO:7 wherein the sequence “CCAGAAGAGG” (3′UTR 10-nucleotide deletion) (SEQ ID NO:8) is deleted therefrom.

The invention also specifically relates to a live attenuated Zika virus (ZIKV) strain as above-described which comprises or consists of a deletion variant of SEQ ID NO:7 wherein the sequence “CTGTGGATCTCCAGAAGAGG” (3′UTR 20-nucleotide deletion) (SEQ ID NO:9) is deleted therefrom.

The invention also specifically relates to an immunogenic composition comprising a live attenuated ZIKV strain as above-described, which further comprises at least one pharmaceutically acceptable carrier or excipient.

The invention also specifically relates to an immunogenic composition comprising a live attenuated ZIKV strain as above-described, which is suitable for parenteral or enteral administration.

The invention also specifically relates to a method of eliciting an immune response in a subject in need thereof comprising administering a prophylactically or therapeutically effective amount of a live attenuated ZIKV strain or immunogenic composition as above-described.

The invention also specifically relates to a method of eliciting an immune response in a subject in need thereof comprising administering a prophylactically or therapeutically effective amount of a live attenuated ZIKV strain or immunogenic composition as above-described, which induces a CD8⁺ T cell response, an antibody response, and/or a cellular immune response against ZIKV.

The invention also specifically relates to a method of eliciting an immune response in a subject in need thereof comprising administering a prophylactically or therapeutically effective amount of a live attenuated ZIKV strain or immunogenic composition as above-described, which produces a neutralizing antibody titer equivalent to that of wildtype ZIKV infection.

The invention also specifically relates to a method of eliciting an immune response in a subject in need thereof comprising administering a prophylactically or therapeutically effective amount of a live attenuated ZIKV strain or immunogenic composition as above-described, wherein the subject is a pregnant female.

The invention also specifically relates to a method of eliciting an immune response in a subject in need thereof comprising administering a prophylactically or therapeutically effective amount of a live attenuated ZIKV strain or immunogenic composition as above-described, in order to prevent congenital ZIKV syndrome.

The invention also specifically relates to a method of eliciting an immune response in a subject in need thereof comprising administering a prophylactically or therapeutically effective amount of a live attenuated ZIKV strain or immunogenic composition as above-described, in order to prevent microcephaly.

The invention also specifically relates to a method of eliciting an immune response in a subject in need thereof comprising administering a prophylactically or therapeutically effective amount of a live attenuated ZIKV strain or immunogenic composition as above-described, wherein at least 1.0×10¹, 1.0×10², 1.0×10³, 1.0×10⁴, 1.0×10⁵, or 1.0×10⁶ IFUs of the live attenuated ZIKV strain is administered to the subject.

The invention also specifically relates to a method of eliciting an immune response in a subject in need thereof comprising administering a prophylactically or therapeutically effective amount of a live attenuated ZIKV strain or immunogenic composition as above-described, wherein the administration of said composition prevents viremia in said subject after subsequent challenge with a wildtype ZIKV strain.

The invention also specifically relates to a method of eliciting an immune response in a subject in need thereof comprising administering a prophylactically or therapeutically effective amount of a live attenuated ZIKV strain or immunogenic composition as above-described, wherein the subject is a human.

### BRIEF DESCRIPTION OF THE SEVERAL VIEWS OF THE DRAWINGS

FIG. 1A-1F contain characterizations of the 3′UTR deletion mutants in cell culture. FIG. 1A provides sequences of the ZIKV 3′UTR deletionsWT (SEQ ID NO:20), 10-del (SEQ ID NO:21), 20-del (SEQ ID NO:22), 30-del-a (SEQ ID NO:23), and 30-del-b (SEQ ID NO: 24). FIG. 1B shows an immunostaining focus assay of mutant viruses. Equal amounts of RNAs (10 μg) transcribed from their corresponding infectious cDNA clones were electroporated into Vero cells. On day 4 or 5 post-transfection, culture fluids from the transfected cells were harvested and quantified for infectious viruses (defined as P0 virus) using an immunostaining focus assay on Vero cells. FIG. 1C demonstrates the replication kinetics of WT and mutant viruses. Vero cells in 24-well plates (2×10⁵ cells per well) were infected with WT and mutant viruses at an MOI of 0.01. Culture fluids were quantified for infectious viruses on days 1 to 5 using the immunostaining focus assay. From left to right for each day, the bars correspond to: WT, 10-del, 20-del, 30-del-a, and 30-del-b. FIG. 1D illustrates a Renilla luciferase reporter replicon construct. FIG. 1E contains a replicon analysis of the 3′UTR deletions. A Renilla luciferase reporter replicon of ZIKV (FIG. 1D) was engineered with various 3′UTR deletions. Equal amounts of replicon WT and mutant RNAs (10 μg) were electroporated into Vero cells. Luciferase signals were measured at the indicated time points. A non-replicative replicon containing an NS5 polymerase-inactive GDD mutation was included as a negative control. The averages of three replicates are presented. Error bars represent standard deviations. RLU, relative light units. The top curve corresponds to WT and the bottom curve corresponds to the GDD control. FIG. 1F shows the interferon-β inhibition of WT and mutant ZIKVs. Vero cells were seeded in 96-well plate (1.5×10⁴ cell per well) one day before interferon treatment and viral infection. The cells were infected at an MOI 0.05 in the presence of IFN-β (55, 167, 500, or 1,500 IU/ml). Viral infection and interferon treatment were initiated at the same time. At 48 h post-infection and interferon-β treatment, viral titers were quantified using the immunostaining focus assay on Vero cells. Percentages of viral titer inhibition are presented in log₁₀ scale. Viral titers without interferon-treatment are set as 100%. Average results of three independent experiments are shown. Error bars represent standard deviations. Symbols ** and *** indicate P values <0.01 and <0.001, respectively. From left to right for each day, the bars correspond to: WT, 10-del, 20-del, 30-del-a, and 30-del-b.

FIG. 2A-2B shows the sequence information of the 3′UTR and the deletion mutants. FIG. 2A depicts the predicted RNA secondary structure of the ZIKV 3′UTR. The stem-loop structure of the 3′UTR of the ZIKV genome is presented as previously reported (References 33, 34). The nucleotide sequence of the shaded stem-loop is shown (representing nucletoides 238 to 307 of SEQ ID NO:1). The deleted sequences for 10-del, 20-del, 30-del-a, and 30-del-b mutants are displayed in blue, magenta, green, and orange, respectively. FIG. 2B shows a sequence alignment of the deleted region (nucleotide position 10,630-10,674) in the 3′UTR. The 10-del nucleotides are indicated. Within the 10-del region, sequence variations are observed for early isolates (P6-740 (SEQ ID NO:30), MR766 (SEQ ID NO:31), and DAK-41525 (SEQ ID NO:32) were isolated in 1966, 1947, and 1984, respectively), while an identical sequence is observed for the strains isolated after 2010 (FSS13025 (SEQ ID NO:25), H/PF2013 (SEQ ID NO:26), PRVABC 59 (SEQ ID NO:27), Natal RGN (SEQ ID NO:28), and ZKV2015 (SEQ ID NO:29)).

FIG. 3 shows an immunofluorescence assay (IFA) of viral protein expression in cells transfected with WT or 3′UTR deletion ZIKV RNA. Vero cells were electroporated with 10 μg of genomic WT or 3′UTR deletion RNA of ZIKV. On day 2 and 3 post-transfection, IFA was performed to examine viral E protein expression using a mouse mAb (4G2) and Alexa Fluor® 488 goat anti-mouse IgG as the primary and secondary antibodies, respectively. Green and blue represent E protein and nuclei (stained with DAPI), respectively. Viral E protein staining is visible for WT and all mutant groups on both days.

FIG. 4A-4C contain a stability analysis of the 3′UTR deletion ZIKVs in cell culture. P0 viruses (derived from the culture fluids of RNA-transfected cells from FIG. 1) were continuously cultured on Vero cells for five rounds (5 days for each round of culture), resulting in P5 viruses. The P5 viruses were then characterized. FIG. 4A shows the results of an immunostaining focus assay. WT and P5 mutant viruses were analyzed using an immunostaining focus assay on Vero cells. For each mutant virus, three independent selections were performed on Vero cells. Representative images of infectious foci for each P5 mutant virus are presented. FIG. 4B shows replication kinetics. Vero cells in 24-well plates (2×10⁵ cells per well) were infected with WT and P5 mutant viruses at an MOI of 0.01. Culture fluids were quantified for infectious viruses on days 1 to 5 using the immunostaining focus assay on Vero cells. From left to right for each day, the bars correspond to: WT, 10-del, 20-del, 30-del-a, and 30-del-b. FIG. 4C shows adaptive mutations in P5 mutant viruses. The complete genomes of P5 mutant viruses were sequenced for each of the three independent selections. The adaptive mutations are indicated by their amino acid positions of indicated genes based on ZIKV FSS13025 strain (GenBank number KU955593.1).

FIG. 5A-5G show a characterization of 3′UTR mutants in the A129 mouse model. FIG. 5A contains an experimental scheme. In two separate experiments, three-week old A129 mice (n=8) were immunized via the S.C. route with 1×10⁴ IFU WT and mutant viruses. The immunized mice were monitored for weight loss, survival, and viremia. FIG. 5B shows the results for weight loss. Weight loss is indicated by percentage using the weight on the day before immunization as 100%. The lowest curve corresponds to WT. FIG. 5C shows the results for survival. The lowest curve corresponds to WT. FIG. 5D shows the results for viremia. Viremias were quantified by an immunostaining focus assay from day 2 to 4 post-infection. From left to right for each day, the bars correspond to: WT, 10-del, 20-del, 30-del-a, and 30-del-b. FIG. 5E shows the pre-challenge neutralization antibody titers. On day 28post-immunization, mouse sera were measured for neutralizing titers using an mCherry ZIKV infection assay (FIG. 6A-6B). FIG. 5F shows the post-challenge viremia. On day 28 post-immunization, mice were challenged with 1×10⁵ PFU parental virus (ZIKV strain FSS13025) via the I.P. route. Viremia on day 2 post-challenge was quantified using the immunostaining focus assay. FIG. 5G shows the post-challenge neutralization antibody titer. On day 28 post-challenge, mouse sera were quantified for neutralizing titers using the mCherry ZIKV infection assay. L.O.D.: limit of detection.

FIG. 6A-6B show the construction of mCherry ZIKV. FIG. 6A shows a schematic genome of an mCherry ZIKV. A DNA fragment (encoding the first 25 amino acids of C gene, the mCherry gene, and the foot-and-mouth virus 2A protein) was in-frame fused with the open-reading-frame of ZIKV genome. FIG. 6B shows the mCherry expression in Vero cells transfected with mCherry ZIKV RNA. The expression of mCherry in transfected Vero cells was analyzed by fluorescent microscopy at the indicated days post-transfection. The mCherry ZIKV was used to estimate antibody neutralization titers of mouse sera, as described in Methods.

FIG. 7A-7C show the efficacy of immunization with 100 IFU 10-del virus. FIG. 7A shows the viremia after immunization with 100 IFU of WT (left bar) or 10-del ZIKV (right bar). Three-week-old A129 mice (n=5) were immunized with 100 IFU WT or 10-del virus via the S.C. route. Viremia was quantified by immunostaining focus assay from day 2 to 6. L.O.D., limit of detection. FIG. 7B shows the pre-challenge neutralization antibody titers. On day 28 post-immunization, mouse sera were quantified for ZIKV neutralizing antibody titers. FIG. 7C shows the viremia after challenge with ZIKV (Puerto Rico strain PRVABC59). On day 28 post-immunization, the mice were challenged with 1×10⁶ IFU of ZIKV via the I.P. route. Viremias were quantified by immunostaining focus assay on day 2 post-challenge.

FIG. 8A-8C show the efficacy of immunization with 10 IFU 10-del virus. FIG. 8A shows the viremia after immunization with 10 IFU of WT or 10-del ZIKV. Three-week-old A129 mice (n=5) were immunized with 10 IFU WT or 10-del virus via the S.C. route. Viremia were quantified by immunostaining focus assay from day 4 to 7. L.O.D., limit of detection. FIG. 8B shows the pre-challenge neutralization antibody titers. Three-week-old A129 mice (n=5) were immunized with 10 IFU 10-del ZIKV and PBS via the S.C. route. On day 28 post-immunization, mouse sera were quantified for ZIKV neutralizing antibody titers. On the same day, the mice were challenged with 1×10⁶ IFU of ZIKV (Puerto Rico strain PRVABC59) via the I.P. route. FIG. 8B shows the viremia after challenge with epidemic ZIKV (Puerto Rico strain PRVABC59). On day 2 post-challenge, viremias were quantified using an immunostaining focus assay. L.O.D.: limit of detection.

FIG. 9A-9D show the T cell responses after primary infection with ZIKV WT or 10-del mutant. A129 mice were infected with 1×10⁴ IFU WT and 10-del viruses. On day 28 post-infection, mouse spleens were harvested. Splenocytes were counted, cultured ex vivo with WT ZIKV for 24 h, and stained for markers (IFN-γ, CD3, and CD4 or CD8). The T cells were gated based on staining for these markers. FIG. 9A shows percentages of CD4⁺IFN-γ⁺ cells and CD8⁺IFN-γ⁺ cells. FIG. 9B shows the average total number of T cell subsets per spleen. Supernatants from the ex vivo culture were harvested on day 2 after WT ZIKV re-stimulation, and measured for IFN-γ and IL-2 production. FIG. 9C shows IFN-γ production. FIG. 9D shows IL-2 production. Data are presented as means ±SEM, n=2−4 per group. *P<0.05 or **P<0.01 difference between the virus-and mock-infected mice.

FIG. 10A-10C show the safety evaluation of 10-del virus. FIG. 10A shows the viral loads in organs of infected A129 mice. Three-week-old A129 mice were immunized with 1×10⁴ IFU of WT and 10-del viruses. Organs from infected mice were collected and homogenized on day 6 and 10 post-infection. The amounts of viruses were quantified on Vero cells using an immunostaining focus assay. The mean results from three animals are presented. Bars denote standard errors. “*” denotes no detectable virus. From left to right on each day, the bars correspond to: heart, lung, liver, spleen, kidney, muscle, brain, testis, and eye. FIG. 10B shows a comparison of neurovirulence of WT and 10-del viruses in CD1 newborn mouse. Groups of one-day-old CD1 mice (n=7−10) were injected via the I.C. route with 10 to 1×10⁴ IFU of WT or 10-del virus. All 10-del virus curves show 100% survival, while WT curves show less than 100% survival. FIG. 10C shows the results of a mosquito infectivity assay. Aedes aegypti were fed with WT or 10-del virus on artificial blood-meals. On day 7 post-feeding, individual engorged, incubated mosquitoes were homogenized and infection was assayed by immunostaining of viral protein expression on inoculated Vero cells (see Methods for details). The number of infected mosquitos and total number of engorged mosquitoes are indicated.

FIG. 11A-11C shows the comparison of viremia and efficacy of P0 and P5 10-del viruses. FIG. 11A shows the viremia after immunization with 100 IFU of P0 or P5 10-del ZIKV. Three-week-old A129 mice (n=5) were immunized with 100 IFU P0 or P5 10-del virus via the S.C. route. Viremia was quantified by immunostaining focus assay from day 4 to 6. L.O.D., limit of detection. FIG. 11B shows pre-challenge neutralization antibody titers. On day 28 post-immunization, mouse sera were quantified for ZIKV neutralizing antibody titers. FIG. 11C shows viremia after challenge with wild-type ZIKV. On day 28 post-immunization, the mice were challenged with 1×10⁶ IFU of an epidemic strain of ZIKV (Puerto Rico strain PRVABC59) via the I.P. route. On day 2 post-challenge, viremias were quantified using an immunostaining focus assay.

FIG. 12A-12B show a sperm count analysis of A129 mice infected with WT or 10-del mutant virus. Male A129 mice were infected with 1×10⁴ IFU of WT and 10-del viruses (n=4 per group). On day 16 p.i., epididymis was harvested for sperm count analysis. FIG. 12A shows total sperm counts. FIG. 12B shows motile sperm counts. One-way ANOVA test was performed to indicate statistical significance among different infection groups. n.s., not significant; ***very significant (p value <0.001); ****extremely significant (p value <0.0001).

FIG. 13A-G shows that ZIKV-3′UTR-Δ10-LAV protects pregnant C57BL/6 mice and their developing fetuses. FIG. 13A shows the scheme of immunization of wild-type (WT) C57BL/6 female mice with 10⁵ FFU of ZIKV-3′UTR-Δ10-LAV (Δ10; n=12) or PBS sham (n=16). FIG. 13B shows experiments wherein serum was collected at day 28 post-immunization and analyzed for neutralizing activity using an mCherry infectious ZIKV. Representative neutralization curves are shown. Error bars denote the standard deviation (SD) of duplicate technical replicates. FIG. 13C shows NT₅₀ values of neutralizing antibodies were measured for individual animals. The dashed lines indicate the limit of detection (L.O.D.) of the assay. FIG. 13D-G shows that at day 35 post-immunization, vaccinated female mice were mated with WT C57BL/6 males. A subset of the female mice developed vaginal plugs. Pregnant mice (n=8 pooled from two independent experiments) were administered 2 mg of anti-Ifnar1 blocking antibody on E5, and one day later (E6), challenged with 10⁵ FFU of a pathogenic, mouse adapted ZIKV Dakar 41519 strain. On E13, animals were euthanized; maternal spleen (FIG. 13D), maternal brain (FIG. 13E), placenta (FIG. 13F), and fetal heads (FIG. 13G) were harvested and quantified for viral RNA levels. Median viral RNA levels are indicated for each group. Asterisks indicate significant differences (Mann-Whitney test: ****, P value <0.0001). All negative samples are plotted at the half value of L.O.D. The results in the Figure are pooled from two independent experiments.

FIG. 14A-I show that ZIKV-3′-UTR-Δ10-LAV protects young A129 male mice against testis infection and injury. FIG. 13A contains the scheme of immunization of 3-week-old A129 male mice with 10⁴ FFU of ZIKV-3′-UTR-Δ10-LAV (Δ10; n=6) or PBS sham (n=4 or 6). At day 28 post-immunization, mice were measured for neutralization antibody titers. On the same day, mice from one sham group and mice from Δ10-immunized group were challenged with 10⁶ FFU of ZIKV-PRVABC59, and viremia was measured at day 2 post-challenge (day 30 post-immunization). At day 49 post-immunization, mice were analyzed for sperm counts and viral load in the testis. FIG. 14A-B shows viremia after immunization with Δ10 vaccine candidate. FIG. 14C contains NT50 values of antibody neutralization at day 28 post-immunization were measured for individual animals in each group. The dashed lines indicate the limit of detection (L.O.D.) of the assay. FIG. 14D shows viremia at day 2 post-challenge (day 30 post-immunization) with ZIKV PRVABC59. FIG. 14E shows viral load in the testis at day 21 post-challenge (day 49 post-immunization). FIG. 14F-G show total (F) and motile (G) sperm counts at day 21 post-challenge. (H-I) Testis weight (H) and representative images of testis (i) from animals from sham, sham with challenge, and Δ10-immunized and challenged groups at day 21 post-challenge. Scale bar, 1 mm. Asterisks indicate significant differences (One-way ANOVA: ****, P value <0.0001; ***, P value <0.001). Non-significant (n.s.), P value >0.5. All negative samples are plotted at the half value of L.O.D. Error bars represent standard deviations.

FIG. 15A-D shows that ZIKV-3′UTR-Δ10-LAV and ZIKV-3′UTR-Δ20-LAV protect rhesus macaques (RM) from ZIKV infection. FIG. 15A shows the scheme of immunization of RM with 10³ FFU of WT ZIKV strain FSS13025 (n=4), ZIKV-3′UTR-Δ10-LAV (Δ10; n=4), ZIKV-3′UTR-Δ20-LAV (Δ20; n=3), or PBS sham (n=2) via the subcutaneous route. FIG. 15B shows viremia measured at day 2, 3, 4, 5, 7, and 10 post-immunization by qRT-PCR. Each colored line represents data from different animals in each group. The dashed line indicates the limit of detection (L.O.D.) of the assay. FIG. 15C shows pre-and post-challenge antibody neutralization titers. On various days post-immunization, sera were measured for neutralizing titers using an mCherry ZIKV infection assay. Red arrows indicate challenge with 10³ FFU of epidemic ZIKV strain PRVABC59 via the subcutaneous route at day 56 post-immunization. The number of animals whose antibody neutralization titers increased by ≥4-fold after challenge is indicated by symbol “↑” for each experimental group. FIG. 15D shows post-challenge viremia. Viremia was measured by qRT-PCR at day 2, 3, 4, 5, 7, and 10 post-challenge. All negative samples are plotted at the half value of L.O.D. Error bars represent standard deviations.

FIG. 16A-H shows a safety evaluation of ZIKV-3′-UTR-Δ20-LAV (Δ20) vaccine candidate. FIG. 16A shows viral loads in organs of infected A129 mice. Three-week-old A129 mice (n=7) were subcutaneously immunized with 10³ FFU of WT ZIKV FSS13025 (left panel) and its derivative Δ20 vaccine candidate (right panel). Organs from infected mice were collected and homogenized at day 6 and 10 post-infection. The amounts of viruses were quantified on Vero cells using a focus forming assay. The mean results from seven animals are presented. Bars denote standard errors. The dashed lines indicate the limit of detection (L.O.D.) of the assay. FIG. 16D-F shows the effect of Δ20 vaccination on the testis. Three-week-old A129 mice (n=5) were subcutaneously infected with 1×10³ FFU of WT ZIKV FSS13025 or Δ20 vaccine candidate. At day 28 post-infection, animals from each group were analyzed for testis weight (FIG. 16B), testis size (FIG. 16C), total sperm counts (FIG. 16D), motile sperm counts (FIG. 16E), and viral RNA load (FIG. 16F). Scale bar, 1 mm. (FIG. 16G) Comparison of neurovirulence of WT ZIKV FSS13025 and Δ20 vaccine candidate in outbred CD-1 mice. One-day-old CD-1 mice (n=7-8 per group) were injected intracranially with 10 to 10⁴ FFU of WT ZIKV or 10³ to 10⁴ FFU of Δ20 vaccine candidate. Survival mice and total infected animals are indicated. (FIG. 16H) Analysis of vector competency. Aedes aegypti were fed on artificial blood-meals spiked with 10⁶ FFU/ml of WT ZIKV FSS13025 or Δ20 vaccine virus. At day 7 post-feeding, individual engorged mosquitoes were assayed for infection by immunostaining of viral protein expression on inoculated Vero cells. The number of infected mosquitos and total number of engorged mosquitoes are indicated. Asterisks indicate significant differences (One-way ANOVA: ***, P value <0.001; **, P value <0.01; *, P value <0.05). Non-significant (n.s.) with P value >0.5. All negative samples are plotted at the half value of L.O.D. Error bars represent standard deviations.

FIG. 17A-C shows the infectious ZIKV burden in placentas and fetal heads from sham or ZIKV-3′UTR-Δ10-LAV-immunized dams. In the pregnancy protection experiment (see details in FIG. 13), at day 7 post-challenge (equivalent to E13), placenta (FIG. 17A) and fetal heads (FIG. 17B) were collected from PBS sham and ZIKV-3′UTR-Δ10-LAV-immunized dams, and quantified for infectious ZIKV using a focus forming assay. Dashed lines indicate limit of detection (L.O.D.) of the assays. Results are pooled from two independent biological experiments, and each symbol represents data from an individual placenta (n=23) or fetus (n=30). (FIG. 17C) Correlation of E13 placenta viral burden with antibody neutralizing NT₅₀ values of ZIKV-3′UTR-Δ10-LAV. P and R² values reflect Pearson correlation tests. All negative samples are plotted at the half value of L.O.D. Error bars represent standard deviations.

FIG. 18A-I contains experiments which show that ZIKV-3′UTR-Δ10-LAV protects adult A129 male mice against testis infection and injury. (A) Scheme of immunization of 15-week-old A129 male mice with 1×10⁴ FFU of ZIKV-3′UTR-Δ10-LAV (Δ10; n=5) or PBS sham (n=5). At day 28 post-immunization, mice were measured for neutralizing antibody titers. On the same day, the mice were challenged with 10⁶ FFU of ZIKV-PRVABC59. Peak viremia was measured at day 2 post-challenge (day 30 post-immunization). At day 49 post-immunization, mice were euthanized and measured for total and motile sperm counts and viral loads in the testis. FIG. 18B shows viremia after ZIKV-3′UTR-Δ10-LAV immunization. FIG. 18C shows NT₅₀ values of antibody neutralization at day 28 post-immunization. Antibody neutralizing titers were measured for individual animals in each group by an mCherry ZIKV. The dashed lines indicate the limit of detection (L.O.D.) of the assay. FIG. 18D shows day 2 post-challenge (day 30 post-immunization) viremia. At day 21 post-challenge, animals from each group were analyzed for testis viral load FIG. 18E shows testis viral load, FIG. 18F shows total sperm counts, FIG. 18G shows motile sperm counts, FIG. 18H shows testis weight, and FIG. 18I shows testis size. Representative images of testis are presented in (i). Scale bar, 1 mm. Asterisks indicate significant differences (One-way ANOVA: *, P value <0.05; **, P value <0.01; ****, P value <0.0001). Non-significant (n.s.), P value >0.5. All negative samples are plotted at the half value of L.O.D. Error bars represent standard deviations.

FIG. 19A-B shows infectious virus in serum of challenged rhesus macaque. Infectious virus in RM serum (viremia) was collected at days 2, 3, 4, 5, 7, and 10 post-immunization (FIG. 19A) or post-challenge (FIG. 19B) was quantified by a focus forming assay. (See detailed experimental scheme in FIG. 15A-D). Dashed lines indicate limit of detection (L.O.D.) of the assays. All negative samples are plotted at the half value of L.O.D.

FIG. 20A-I shows that ZIKV-3′-UTR-Δ20-LAV protects young A129 male mice against testis infection and injury. FIG. 20A shows the scheme of immunization of 3-week-old A129 male mice with 10³ FFU of ZIKV-3′-UTR-Δ20-LAV (Δ20; n=6) or PBS sham (n=4 or 6). FIG. 20B shows viremia post immunization. At day 28 post-immunization, immunized mice were measured for neutralization antibody titers. At the same day, mice from one sham group and mice from Δ20-immunized group were challenged with 10⁶ FFU of ZIKV-PRVABC59. FIG. 20D shows viremia measured at day 2 post-challenge (day 30 post-immunization). FIGS. 20E&F show that at day 49 post-immunization, mice were analyzed for sperm counts and viral loads in testis. FIG. 20C shows NT₅₀ values of antibody neutralization at day 28 post-immunization were measured for individual animals in each group. The dashed lines indicate the limit of detection (L.O.D.) of the assay. FIG. 20D shows day 2 post-challenge (day 30 post-immunization) viremia. FIGS. 20E & F respectively show total (E) and motile (F) sperm counts at day 21 post-challenge (equivalent to day 49 post-immunization). FIG. 20G shows testis weight from animals from sham, sham with challenge, and Δ20-immunized and challenged groups at day 21 post-challenge. FIG. 20H shows viral load in testis at day 21 post-challenge. FIG. 20I contains representative images of testis harvested at day 21 post-challenge. Scale bar, 1 mm. Asterisks indicate significant differences (One-way ANOVA: ****, P value <0.0001). Non-significant (n.s.) with P value >0.5. All negative samples are plotted at the half value of L.O.D. Error bars represent standard deviations.

FIG. 21 contains a summary of experiments which evaluated the stability of ZIKV-3′UTR-Δ20-LAV in cell culture. P0 viruses (derived from the culture fluids of RNA-transfected cells) were continuously cultured on Vero cells for five rounds (5 days for each round of culture), resulting in P5 viruses. The complete genomes of P5 mutant viruses were sequenced. All P5 viruses retained the 20-nucleotide deletion in the 3′UTR. In addition, several adaptive mutations are recovered; these mutations are presented by their amino acid positions of indicated genes based on ZIKV FSS13025 strain (GenBank number KU955593.1). Results from three independent passages are presented.

FIG. 22 depicts the results of experiments wherein 5 micrograms of a Zika DNA plasmid according to the invention was transfected into Vero cells through electroporation. Culture fluids were collected from day 1 to 5. Infectious viral titers were measured by plaque assay on Vero cells.

FIG. 23 shows the yield of ZIKV DNA vaccine candidates on Vero cells. Five micrograms of indicated DNA plasmid was transfected into Vero cells through electroporation. Culture fluids were collected from day 1 to 5. Infectious viral titers were measured by plaque assay on Vero cells.

### DETAILED DESCRIPTION OF THE INVENTION

The present invention in general relates to the construction and characterization of a novel live attenuated Zika virus (ZIKV) strain having one or more deletions in the 3′ untranslated region (3′UTR). These ZIKV deletion mutants may have reduced RNA production and increased susceptibility to interferon-β inhibition, and thus can be utilized as effective live attenuated vaccines against ZIKV. Particularly we show herein that live-attenuated ZIKV vaccine candidates containing deletions in the 3′ untranslated region of the ZIKV genome (ZIKV-3′UTR-LAV) prevent viral transmission during pregnancy and testis damage in mice, as well as inhibiting infection in non-human primates. We also demonstrate a desirable safety profile of the vaccine candidates. Our results suggest that ZIKV-3′UTR-LAV potentially may be used to vaccinate humans against Zika virus infection.

Moreover, as evidenced by the results disclosed herein mutated live attenuated Zika virus strains according to the invention and compositions containing same may be used in treating or providing immunoprotection against infections elicited by ZIKV, including congenital ZIKV syndrome, microcephaly, and Guillan-Barré syndrome (GBS).

The present invention provides a vaccine which may be used to prevent viremia in pregnant women and travelers to epidemic/endemic regions to avert congenital ZIKV syndrome and which may also be useful to suppress epidemic transmission. The ZIKV strain of the invention is a live-attenuated vaccine candidate that contains a deletion or “Δ” in the 3′ untranslated region of ZIKV genome, preferably a 10-nucleotide deletion (10-del ZIKV) or a 20-nucleotide deletion (20-del ZIKV) and more preferably comprising or consisting of the Zika strains having the sequences in Appendix A modified as set forth in Appendix B. The 10-del ZIKV is highly attenuated, immunogenic, and protective in the A129 mouse model. A single dose of 10 IFU of 10-del ZIKV elicited a high level of neutralizing antibodies and completely prevented viremia after challenge. Besides the antibody response, the immunized mice also developed a robust T cell response. Intracranial inoculation of one-day-old CD1 mice with 1×10⁴ IFU of 10-del ZIKV caused no detectable disease, whereas infections with 10 IFU of wild-type ZIKV were lethal. Mechanistically, the 10-del ZIKV attenuated its virulence through decreased viral RNA synthesis and increased sensitivity to type-I interferon inhibition. The attenuated 10-del ZIKV was incompetent in infecting mosquitoes, representing an additional safety feature for use in non-endemic regions. Collectively, the safety and efficacy results warrant further development of this promising live-attenuated ZIKV vaccine candidate.

The live attenuated ZIKV strains of the invention may further comprise additional mutations to the ZIKV genome. A mutation can be, but is not limited to, a deletion of non-coding or coding nucleotides, a deletion of one or more amino acids, an addition of one or more amino acids, a substitution (conserved or non-conserved) of one or more amino acids or a combination thereof. ZIKV can be mutated, e.g., using deletions to the 3′UTR, such that the infectivity of ZIKV is reduced. In certain embodiments, the infectivity of ZIKV is reduced by a factor of at least 5, 10, 50, 100, 500, 10, 5×10³, 10₄, 5×10⁴, 10⁵, 5×10⁵, or at least 10⁶.

Additionally, ZIKV can be mutated, e.g., having deletions to the 3′UTR and/or using point mutations, such that the rate of replication of the recombinant virus is reduced or increased. The rate of replication can be determined by any standard technique known to the skilled artisan. The rate of replication is represented by the growth rate of the virus and can be determined by plotting the viral titer over the time post infection. The viral titer can be measured by any technique known to the skilled artisan. In certain embodiments, a suspension containing the virus is incubated with cells that are susceptible to infection by the virus including, but not limited to, Vero cells, LLC-MK-2 cells, Hep-2 cells, LF 1043 (HEL) cells, MRC-5 cells, WI-38 cells, tMK cells, 293 T cells, QT 6 cells, QT 35 cells, or chicken embryo fibroblasts (CEF). Subsequent to the incubation of the virus with the cells, the number of infected cells is determined. In certain specific embodiments, the virus comprises a reporter gene. Thus, the number of cells expressing the reporter gene is representative of the number of infected cells. In a specific embodiment, the virus comprises a heterologous nucleotide sequence encoding mCherry, and the number of cells expressing mCherry, i.e., the number of cells infected with the virus, is determined using FACS.

The assays described herein may be used to assay viral titre over time to determine the growth characteristics of the virus. In a specific embodiment, the viral titre is determined by obtaining a sample from the infected cells or the infected subject, preparing a serial dilution of the sample and infecting a monolayer of cells that are susceptible to infection with the virus at a dilution of the virus that allows for the emergence of single plaques. The plaques can then be counted and the viral titre express as plaque forming units per milliliter of sample. In a specific embodiment of the invention, the growth rate of a virus of the invention in a subject is estimated by the titer of antibodies against the virus in the subject. Without being bound by theory, the antibody titer in the subject reflects not only the viral titer in the subject but also the antigenicity. If the antigenicity of the virus is constant, the increase of the antibody titer in the subject can be used to determine the growth curve of the virus in the subject. In a preferred embodiment, the growth rate of the virus in animals or humans is best tested by sampling biological fluids of a host at multiple time points post-infection and measuring viral titer.

The expression of heterologous gene sequence in a cell culture system or in a subject can be determined by any technique known to the skilled artisan. In certain embodiments, the expression of the heterologous gene is measured by quantifying the level of the transcript. The level of the transcript can be measured by Northern blot analysis or by RT-PCR using probes or primers, respectively that are specific for the transcript. The transcript can be distinguished from the genome of the virus because the virus is in the antisense orientation whereas the transcript is in the sense orientation. In certain embodiments, the expression of the heterologous gene is measured by quantifying the level of the protein product of the heterologous gene. The level of the protein can be measured by Western blot analysis using antibodies that are specific to the protein.

The invention provides a live attenuated ZIKV strain comprising a deletion of one or more nucleotides in the 3′UTR of the ZIKV genome. In some embodiments, the ZIKV strain of the invention may comprise a 1-nucleotide deletion, a 2-nucleotide deletion, a 3-nucleotide deletion, a 4-nucleotide deletion, a 5-nucleotide deletion, a 6-nucleotide deletion, a 7-nucleotide deletion, an 8-nucleotide deletion, a 9-nucleotide deletion, a 10-nucleotide deletion, an 11-nucleotide deletion, a 12-nucleotide deletion, a 13-nucleotide deletion, a 14-nucleotide deletion, a 15-nucleotide deletion, a 16-nucleotide deletion, a 17-nucleotide deletion, an 18-nucleotide deletion, a 19-nucleotide deletion, a 20-nucleotide deletion, 21-nucleotide deletion, a 22-nucleotide deletion, a 23-nucleotide deletion, a 24-nucleotide deletion, a 25-nucleotide deletion, a 26-nucleotide deletion, a 27-nucleotide deletion, a 28-nucleotide deletion, a 29-nucleotide deletion, a 30-nucleotide deletion, 31-nucleotide deletion, a 32-nucleotide deletion, a 33-nucleotide deletion, a 34-nucleotide deletion, a 35-nucleotide deletion, a 36-nucleotide deletion, a 37-nucleotide deletion, a 38-nucleotide deletion, a 39-nucleotide deletion, a 40-nucleotide deletion, a 41-nucleotide deletion, a 42-nucleotide deletion, a 43-nucleotide deletion, a 44-nucleotide deletion, a 45-nucleotide deletion, a 46-nucleotide deletion, a 47-nucleotide deletion, a 48-nucleotide deletion, a 49-nucleotide deletion, or a 50-nucleotide deletion in the 3′UTR of the ZIKV genome.

The live attenuated ZIKV strains of the present invention, nucleotide sequences encoding the same, vectors encoding the same, and cells comprising nucleotide sequences encoding said strains may be further modified, engineered, optimized, or appended in order to provide or select for various features. In addition to deletions within the 3′UTR, the attenuated virus may also contain other mutations including, but not limited to, replacing a gene of the human virus with the analogous gene of a virus of a different species, of a different subgroup, or of a different variant.

In some embodiments, other mutations may be introduced into the virus (e.g., missense mutations) can be introduced into the C, prM, E, NS1, NS2A, NS2B, NS3, NS4A, NS4B, or NS5 proteins of the recombinant virus. Also, the mutations may include additions, substitutions, deletions, or combinations thereof. For example a deletion mutation in any of the C, prM, E, NS1, NS2A, NS2B, NS3, NS4A, NS4B, or NS5 proteins may be introduced. In other embodiments, a missense mutation may be introduced which results in a cold-sensitive mutation or a heat-sensitive mutation. In some embodiments, major phosphorylation sites of viral protein may be removed.

In other embodiments, deletions are introduced into the genome of the recombinant virus. In more specific embodiments, a deletion can be introduced into the C, prM, E, NS1, NS2A, NS2B, NS3, NS4A, NS4B, or NS5 proteins of the recombinant virus.

In certain embodiments, the intergenic region of the recombinant virus is altered. In one embodiment, the length of the intergenic region is altered. In another embodiment, the intergenic regions may be shuffled from 5′ to 3′ end of the viral genome. In other embodiments, the genome position of a gene or genes of the recombinant virus can be changed.

In certain embodiments, attenuation of the virus is further enhanced by replacing a gene of the wild type virus with a gene of a virus of a different species, of a different subgroup, or of a different variant.

The attenuated phenotypes of a recombinant virus of the invention can be tested by any method known to the artisan. A candidate virus can, for example, be tested for its ability to infect a host or for the rate of replication in a cell culture system. In certain embodiments, growth curves at different temperatures are used to test the attenuated phenotype of the virus. For example, an attenuated virus is able to grow at 35° C., but not at 39° C. or 40° C. In certain embodiments, different cell lines can be used to evaluate the attenuated phenotype of the virus. For example, an attenuated virus may only be able to grow in monkey cell lines but not the human cell lines, or the achievable virus titers in different cell lines are different for the attenuated virus. In certain embodiments, viral replication in the respiratory tract of a small animal model, including but not limited to, hamsters, cotton rats, mice and guinea pigs, is used to evaluate the attenuated phenotypes of the virus. In other embodiments, the immune response induced by the virus, including but not limited to, the antibody titers (e.g., assayed by plaque reduction neutralization assay or ELISA) is used to evaluate the attenuated phenotypes of the virus. In certain embodiments, the ability of the recombinant virus to elicit pathological symptoms in an animal model can be tested. A reduced ability of the virus to elicit pathological symptoms in an animal model system is indicative of its attenuated phenotype. In a specific embodiment, the candidate viruses are tested in a monkey model for nasal infection, indicated by mucous production.

Various assays can be used to test the safety of a vaccine. For example, sucrose gradients and neutralization assays can be used to test the safety. A sucrose gradient assay can be used to determine whether a heterologous protein is inserted in a virion. If the heterologous protein is inserted in the virion, the virion should be tested for its ability to cause symptoms even if the parental strain does not cause symptoms. Without being bound by theory, if the heterologous protein is incorporated in the virion, the virus may have acquired new, possibly pathological, properties.

Attenuated virus produced according to the invention will be used to confer prophylactic or therapeutic protection in susceptible hosts against ZIKV infection, e.g., to treat or prevent ZIKV infection and/or to prevent congenital ZIKV syndrome or GBS. The attenuated ZIKV strain may be formulated using known techniques for formulating attenuated viral vaccines or immunogenic compositions of viral vaccines.

In one embodiment, the 3′UTR of the ZIKV strain of the invention comprises the nucleic acid sequence of the 3′UTR of the 10-del mutant ZIKV strain, SEQ ID NO: 2.

In one embodiment, the 3′UTR of the ZIKV strain of the invention comprises the nucleic acid sequence of the 3′UTR of the 20-del mutant ZIKV strain, SEQ ID NO: 3.

In one embodiment, the 3′UTR of the ZIKV strain of the invention comprises the nucleic acid sequence of the 3′UTR of the 30-del-a mutant ZIKV strain, SEQ ID NO: 4.

In one embodiment, the 3′UTR of the ZIKV strain of the invention comprises the nucleic acid sequence of the 3′UTR of the 30-del-b mutant ZIKV strain, SEQ ID NO: 5.

In some exemplary embodiments the ZIKV strain of the invention comprises or consists of the sequences set forth in Appendix A modified as set forth in Appendix B.

In some exemplary embodiments immunogenic compositions are provided containing a therapeutically or prophylactically effective amount of a ZIKV strain which comprises or consists of the sequences set forth in Appendix A modified as set forth in Appendix B.

In some exemplary embodiments individuals in need thereof are administered therapeutically or prophylactically effective amount of a ZIKV strain which comprises or consists of the sequences set forth in Appendix A modified as set forth in Appendix B.

### Administration

The immunogenic compositions of the present invention may be administered in a number of ways depending upon whether local or systemic treatment is desired. In exemplary embodiments administration may be topical, parenteral, or enteral.

The pharmaceutical compositions of the invention are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intrasynovial injection or infusions; and kidney dialytic infusion techniques.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

The terms “oral”, “enteral”, “enterally”, “orally”, “non-parenteral”, “non-parenterally”, and the like, refer to administration of a compound or composition to an individual by a route or mode along the alimentary canal. Examples of “oral” routes of administration of a vaccine composition include, without limitation, swallowing liquid or solid forms of a vaccine composition from the mouth, administration of a vaccine composition through a nasojejunal or gastrostomy tube, intraduodenal administration of a vaccine composition, and rectal administration, e.g., using suppositories for the lower intestinal tract of the alimentary canal.

Preferably, the formulated virus containing composition is suitable for intranasal, injection, topical or oral administration, for example as a dried stabilized powder for reconstitution in a suitable buffer prior to administration or in an aerosol composition. In a preferred embodiment, the composition is intranasally administered.

Pharmaceutical compositions and formulations for topical administration may include transdermal patches, ointments, lotions, creams, gels, drops, suppositories, sprays, liquids, semi-solids, monophasic compositions, multiphasic compositions (e.g., oil-in-water, water-in-oil), foams microsponges, liposomes, nanoemulsions, aerosol foams, polymers, fullerenes, and powders (see, e.g., Reference 35, Taglietti et al. (2008) Skin Ther. Lett. 13:6-8). Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal, or intraventricular administration may include sterile aqueous solutions that may also contain buffers, diluents and other suitable additives such as, but not limited to, penetration enhancers, carder compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, aerosols, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. Agents that enhance uptake of oligonucleotides at the cellular level may also be added to the pharmaceutical and other compositions of the present invention. For example, cationic lipids, such as lipofectin, cationic glycerol derivatives, and polycationic molecules, such as polylysine, also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

The compositions of the present invention may include excipients known in the art. Examples of excipients used for vaccine formulation such as adjuvants, stabilizers, preservatives, and trace products derived from vaccine manufacturing processes include but are not limited to: Aluminum Hydroxide, Amino Acids, Benzethonium Chloride, Formaldehyde or Formalin, Inorganic Salts and Sugars, Vitamins, Asparagine, Citric Acid, Lactose, Glycerin, Iron Ammonium Citrate, Magnesium Sulfate, Potassium Phosphate, Aluminum Phosphate, Ammonium Sulfate, Casamino Acid, Dimethyl-betacyclodextrin, 2-Phenoxyethanol, Bovine Extract, Polysorbate 80, Aluminum Potassium Sulfate, Gelatin, Sodium Phosphate, Thimerosal, Sucrose, Bovine Protein, Lactalbumin Hydrolysate, Formaldehyde or Formalin, Monkey Kidney Tissue, Neomycin, Polymyxin B, Yeast Protein, Aluminum Hydroxyphosphate Sulfate, Dextrose, Mineral Salts, Sodium Borate, Soy Peptone, MRC-5 Cellular Protein, Neomycin Sulfate, Phosphate Buffers, Polysorbate, Bovine Albumin or Serum, DNA, Potassium Aluminum Sulfate, Amorphous Aluminum Hydroxyphosphate Sulfate, Carbohydrates, L-histidine, Beta-Propiolactone, Calcium Chloride, Neomycin, Ovalbumin, Potassium Chloride, Potassium Phosphate, Sodium Phosphate, Sodium Taurodeoxychoalate, Egg Protein, Gentamicin, Hydrocortisone, Octoxynol-10,.α-Tocopheryl Hydrogen Succinate, Sodium Deoxycholate, Sodium Phosphate, Beta-Propiolactone, Polyoxyethylene 910, Nonyl Phenol (Triton N-101, Octoxynol 9), Octoxinol-9 (Triton X-100), Chick Kidney Cells, Egg Protein, Gentamicin Sulfate, Monosodium Glutamate, Sucrose Phosphate Glutamate Buffer Calf Serum Protein, Streptomycin, Mouse Serum Protein, Chick Embryo Fibroblasts, Human Albumin, Sorbitol, Sodium Phosphate Dibasic, Sodium Bicarbonate, Sorbitol, Sucrose, Potassium Phosphate Monobasic, Potassium Chloride, Potassium Phosphate Dibasic, Phenol, Phenol Red (Phenolsulfonphthalein), Amphotericin B, Chicken Protein, Chlortetracycline, Ethylenediamine-Tetraacetic Acid Sodium (EDTA), Potassium Glutamate, Cell Culture Media, Sodium Citrate, Sodium Phosphate Monobasic Monohydrate, Sodium Hydroxide, Calcium Carbonate, D-glucose, Dextran, Ferric (III) Nitrate, L-cystine, L-tyrosine, Magnesium Sulfate, Sodium Hydrogenocarbonate, Sodium Pyruvate, Xanthan, Peptone, Disodium Phosphate, Monosodium Phosphate, Polydimethylsilozone, Hexadecyltrimethylammonium Bromide Ascorbic Acid, Casein, Galactose, Magnesium Stearate, Mannitol, Hydrolyzed Porcine Gelatin, Freund's emulsified oil adjuvants (complete and incomplete), Arlacel A, Mineral oil, Emulsified peanut oil adjuvant (adjuvant 65), Corynebacterium granulosum-derived P40 component, Lipopolysaccharide, Mycobacterium and its components, Cholera toxin, Liposomes, Immunostimulating complexes (ISCOMs), Squalene, and Sodium Chloride.

The vaccine or immunogenic composition may be used in the vaccination of a mammalian host, particularly a human, nonhuman primate, ape, monkey, horse, cow, carabao, goat, duck, bat, or other suitable non-human host. A dosage may comprise at least 10 IFU, 10¹ IFU, 10² IFU, 10³ IFU, 10⁴ IFU, 5×10⁴ IFU, 10⁵ IFU, 5×10⁵ IFU, 10⁶ IFU, 5×10⁶IFU, 10⁷ IFU, 5×10⁷ IFU, 10⁸ IFU, or 5×10⁸ IFU of said live attenuated ZIKV strain. In some instances the subject may be immunocompromised or may have another condition, e.g., may be pregnant.

### Definitions

The “3′UTR” or “3′ untranslated region” or “three prime untranslated region” of the ZIKV genome corresponds to the section of RNA that immediately follows the translation termination codon of the genomic polyprotein.

An “adjuvant” refers to a substance that enhances an immune response, e.g., an antibody or cell-mediated immune response against a specific agent, e.g., an antigen, or an infectious agent.

An “attenuated” or “live attenuated” virus strain refers a mutated or modified or recombinant virus having reduced or no virulence or propensity to cause a disease or infection normally associated with the “wild-type” or “unmodified” (or in this case “non-mutated”) virus.

An “attenuated” or “live attenuated” ZIKV strain, in particular, refers to a ZIKV strain that has been modified to have reduced or no virulence or propensity to cause a disease or infection which is normally associated with a “wild-type” or “unmodified” or “non-mutated” virus, in particular congenital ZIKV syndrome or GBS. More particularly, this includes “attenuated” ZIKV strains that are “modified” or “altered” or “mutated” to have one or more deletions in the 3′UTR of the ZIKV genome, e.g., a 10-nucleotide, 20-nucleotide, or 30-nucleotide deletion in the 3′UTR, preferably a 10-nucleotide deletion. The deletions may not disrupt RNA translation. The deletions may slow RNA production and increase interferon-β susceptibility. Such live attenuated ZIKV strain elicits immunoprotection against the virus, i.e., maintains an important immunogenic epitope.

“Heterologous” means derived from a genetically distinct entity from the rest of the entity to which it is being compared. For example, a polynucleotide may be placed by genetic engineering techniques into a plasmid or vector derived from a different source, and is a heterologous polynucleotide. A promoter removed from its native coding sequence and operatively linked to a coding sequence other than the native sequence is a heterologous promoter. The polynucleotides of the invention may comprise additional sequences, such as additional encoding sequences within the same transcription unit, controlling elements such as promoters, ribosome binding sites, 5′UTR, 3′UTR, transcription terminators, polyadenylation sites, additional transcription units under control of the same or a different promoter, sequences that permit cloning, expression, homologous recombination, and transformation of a host cell, and any such construct as may be desirable to provide embodiments of this invention.

An “immunogenic composition” herein refers to a composition containing a live attenuated ZIKV strain according to the invention which elicits an immune response in a susceptible host, e.g., an antibody, Th1 or cellular (e.g., T cell-mediated) immune response.

An “isolated” biological component (such as an isolated bacterium or nucleic acid) refers to a component that has been substantially separated or purified away from its environment or other biological components in the cell of the organism in which the component naturally occurs, for instance, other chromosomal and extra-chromosomal DNA and RNA, proteins, and organelles. Nucleic acids and proteins that have been “isolated” include nucleic acids and proteins purified by standard purification methods. The term also embraces nucleic acids and proteins prepared by recombinant technology as well as chemical synthesis.

The term “nucleic acid” and “polynucleotide” refer to RNA or DNA that is linear or branched, single or double stranded, or a hybrid thereof. The term also encompasses RNA/DNA hybrids. The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracyl, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support. The polynucleotides can be obtained by chemical synthesis or derived from a microorganism. The term “gene” is used broadly to refer to any segment of polynucleotide associated with a biological function. Thus, genes include introns and exons as in genomic sequence, or just the coding sequences as in cDNAs and/or the regulatory sequences required for their expression. For example, gene also refers to a nucleic acid fragment that expresses mRNA or functional RNA, or encodes a specific protein, and which includes regulatory sequences.

A “pharmaceutically acceptable carrier” or “excipient” refers to compounds or materials conventionally used in immunogenic or vaccine compositions during formulation and/or to permit storage.

“Prophylactically effective amount” of a live attenuated ZIKV strain according to the invention refers to an amount sufficient to prevent or reduce the incidence of infection in a susceptible host.

The term “recombinant” means a polynucleotide with semisynthetic, or synthetic origin which either does not occur in nature or is linked to another polynucleotide in an arrangement not found in nature.

A “susceptible host” herein refers to a host or animal that may be infected by ZIKV. Such hosts include humans or animals, e.g., a human, nonhuman primate, ape, monkey, horse, cow, carabao, goat, duck, bat, or other suitable non-human host.

“Therapeutically effective amount” of a live attenuated ZIKV strain according to the invention refers to an amount sufficient to treat ZIKV infection or a disease associated therewith in a susceptible host.

A “vaccine” composition herein refers to a composition containing a live attenuated ZIKV strain according to the invention which elicits a therapeutic or prophylactic immune response against ZIKV.

“ZIKV infection” or “infection elicted by ZIKV” herein refers to the infection of a susceptible host with ZIKV and diseases associated therewith, including congenital ZIKV syndrome and Guillan-Barré syndrome (GBS).

The following examples are offered to illustrate, but not to limit, the claimed invention.

### EXAMPLES

### Example 1: Generating Live Attenuated ZIKV Strains With 3′UTR Deletions

### Materials and Methods

Viruses: The ZIKV Cambodian strain FSS13025 (GenBank number KU955593.1) was generated from an infectious cDNA clone pFLZIKV as described previously (Reference 10). All the cell lines are tested negative for mycoplasma.

Plasmid construction. Standard molecular biology procedures were performed for all plasmid constructions. Standard overlap PCR was performed to amplify the DNA fragment between unique restriction enzyme sites EcoRI and ClaI using corresponding primer pairs. The DNA fragment containing 3′UTR deletion mutations were individually introduced into the pFLZIKV and pZIKV Rep (replicon cDNA plasmid, Reference 11) through EcoRI and ClaI. All the constructs were verified by DNA sequencing. Primer sequences are available upon request. All restriction enzymes were purchased from New England BioLabs (Ipswitch, MA).

### Results

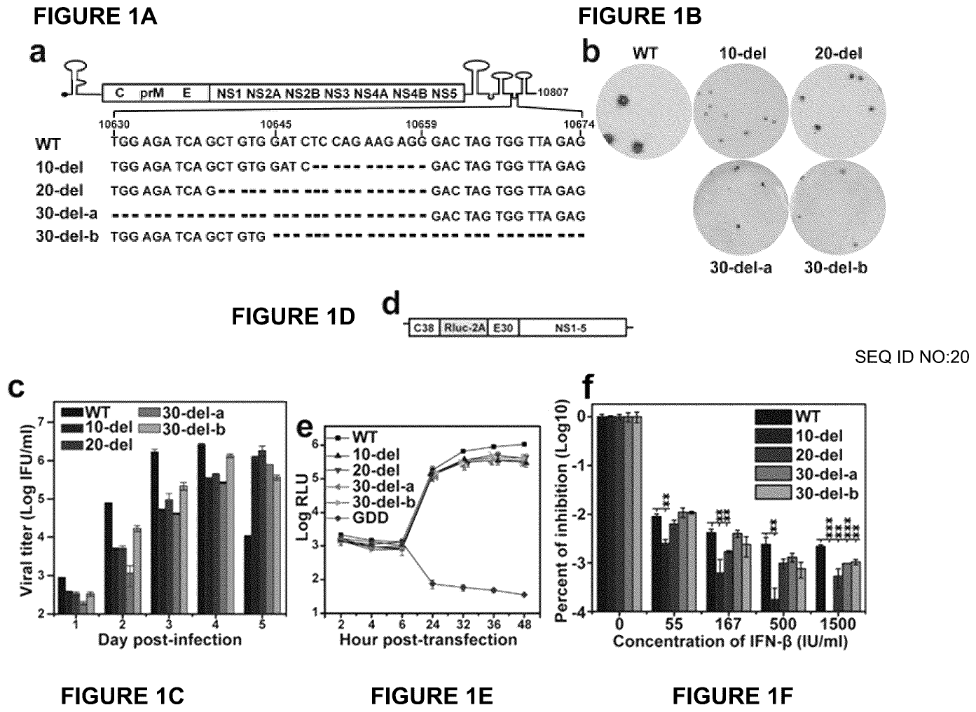

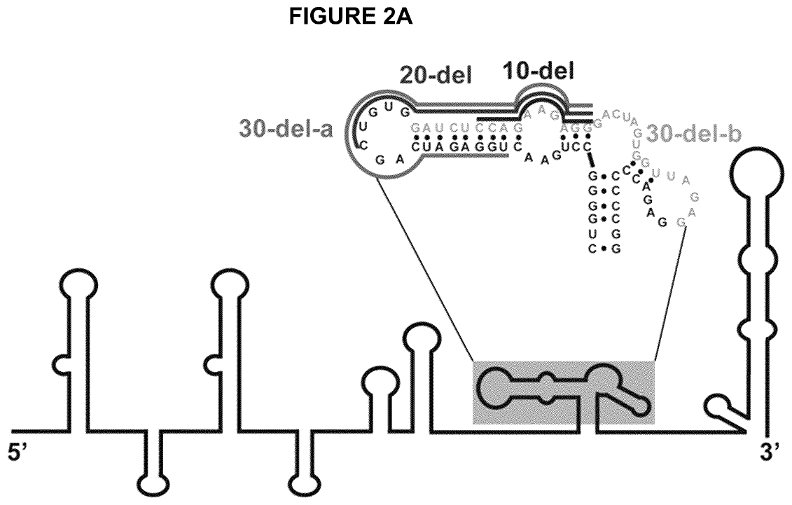

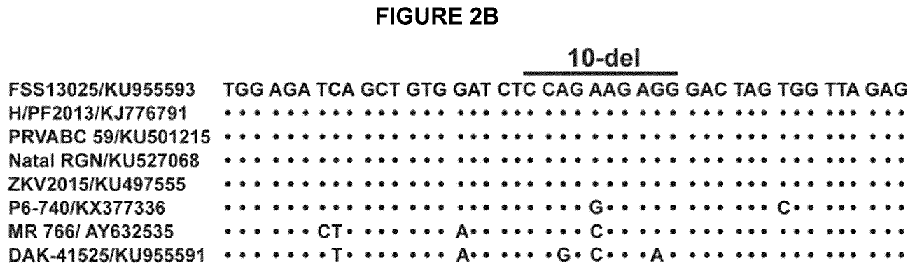

We chose to pursue a live-attenuated vaccine to capitalize on its advantages of single-dose immunization, a rapid and robust immune response, and long-lived protection. We attenuated wild-type (WT) ZIKV through deletion of a portion of the 3′ untranslated region (3′UTR) of the viral genome, as has been successfully used to develop a DENV vaccine currently in a phase III clinical trial (Reference 9). Using an infectious cDNA clone of the ZIKV Cambodian strain FSS13025 (Reference 10) (which is closely related to strains now circulating in the Americas), we prepared a panel of recombinant viruses containing distinct 3′UTR deletions (FIG. 1A). Mutants 10-del, 20-del, 30-del-a, and 30-del-b contained overlapping 10-to-30-nucleotide deletions, which were expected to change the local secondary structure of the viral 3′UTR (FIG. 2).

### Example 2: Replication and IFN-β Inhibition Analysis of ZIKV 3′UTR Deletion Mutants

### Materials and Methods

Cells and antibodies. Vero cells were purchased from the American Type Culture Collection (ATCC, Bethesda, MD), and maintained in a high glucose Dulbecco modified Eagle medium (DMEM) (Invitrogen, Carlsbad, CA) supplemented with 10% fetal bovine serum (FBS) (HyClone Laboratories, Logan, UT) and 1% penicillin/streptomycin (Invitrogen, Carlsbad, CA) at 37° C. with 5% CO₂. The following antibodies were used in this study: a mouse monoclonal antibody (mAb) 4G2 cross-reactive with flavivirus E protein (ATCC), ZIKV-specific HMAF (hyper-immune ascitic fluid), World Reference Center of Emerging Viruses and Arboviruses (WRCEVA) at the University of Texas Medical Branch], Anti-Mouse IgG (H+L) Antibody Horseradish Peroxidase-labeled (KPL, Gaithersburg, MD), and goat anti-mouse IgG conjugated with Alexa Fluor 488 (Thermo Fisher Scientific).

RNA transcription and transfection. Full-genome ZIKV, mCherry ZIKV, and replicon RNAs were in vitro transcribed using a T7 mMessage mMachine kit (Ambion, Austin, TX) from cDNA plasmids pre-linearized by ClaI. The RNA was precipitated with lithium chloride, washed with 70% ethanol, re-suspended in RNase-free water, quantitated by spectrophotometry, and stored at −80° C. in aliquots. The RNA transcripts (10 μg) were electroporated into Vero cells following a protocol described previously (Reference 31).

Indirect immunofluorescence assays (IFA). Vero cells were electroporated with 10 μg of genomic WT or 3′UTR deletion RNA of ZIKV and grown in an 8-well Lab-Tek chamber slide (Thermo Fisher Scientific, Waltham, MA). On day 2 and 3 post-transfection, the cells were fixed in 100% methanol at −20° C. for 15 min. After 1 h incubation in a blocking buffer containing 1% FBS and 0.05% Tween-20 in PBS, the cells were treated with a mouse monoclonal antibody 4G2 for 1 h and washed three times with PBS (5 min for each wash). The cells were then incubated with Alexa Fluor® 488 goat anti-mouse IgG for 1 h in blocking buffer, after which the cells were washed three times with PBS. The cells were mounted in a mounting medium with DAPI (4′, 6-diamidino-2-phenylindole; Vector Laboratories, Inc.). Fluorescence images were observed under a fluorescence microscope equipped with a video documentation system (Olympus).

Immunostaining focus assay of mutant viruses. Equal amounts of RNAs (10 μg) transcribed from their corresponding infectious cDNA clones were electroporated into Vero cells. On day 4 or 5 post-transfection, culture fluids form the transfected cells were harvested and quantified for infectious viruses (defined as P0 virus) using an immunostaining focus assay on Vero cells.

Immunostaining focus assay and immunostaining. Viral samples were ten-fold serially diluted six times in DMEM. For each dilution, 100 μl sample was added to a 24-well plate containing Vero cells at about 90% confluency. The infected cells were incubated for 1 h and swirled every 15 min to ensure complete coverage of the monolayer for even infection. After 1 h incubation, 0.5 ml of methyl cellulose overlay containing 2% FBS 1% penicillin/streptomycin was added to each well. The plate was incubated at 37° C. for four days. Following the incubation, methyl cellulose overlay was removed and 0.5 ml methanol-acetone (1:1) solution was added into each well and incubated at room temperature for 15 min. Fixation solution was aspirated and plates were allowed to air dry, then washed three times with PBS and incubated in blocking buffer (PBS supplemented with 3% FBS), followed by 1 h incubation with ZIKV-specific HMAF. Plates were washed three times with PBS followed by an hour-long incubation with a secondary antibody conjugated to horseradish peroxidase (KPL, Gaithersburg, MD). Detection proceeded with the addition of aminoethylcarbazole substrate (ENZO Life sciences, Farmingdale, MA) prepared according to the vendor's instructions.

Luciferase assay. The luciferase assay was performed as previously reported (Reference 11). Briefly, Vero cells transfected with WT or mutant ZIKV replicon RNAs (10 μg) were seeded in a 12-well plate. At various time points, the cells were washed once with phosphate-buffered saline (PBS) and lysed using cell lysis buffer (Promega, Madison, WI). The cells were scraped from plates and stored at −80° C. The luciferase signals were measured by Cytation 5 (Biotek) according to the manufacturer's instructions.

Replication curves. Subconfluent Vero cells in 24-well plates (2×10⁵ cells per well) were infected with WT or mutant P0 ZIKV at a multiplicity of infection (MOI) of 0.01 in triplicate wells. Virus stocks were diluted in DMEM containing 2% FBS and 1% penicillin/streptomycin. One hundred microliters of virus were added to each well of the 12-well plates. After 1 h attachment (5% CO₂ at 37° C.), the inocula were removed, monolayers were washed three times with PBS, and 1 ml DMEM medium containing 2% FBS and 1% penicillin/streptomycin was added to each well. Culture fluids were quantified for infectious viruses on days 1 to 5 using the immunostaining focus assay.

Replicon analysis of the 3′UTR deletions. A Renilla luciferase reporter replicon of ZIKV was engineered with various 3′UTR deletions. Equal amounts of replicon WT and mutant RNAs (10 μg) were electroporated into Vero cells. Luciferase signals were measured at several time points. A non-replicative replicon containing an NS5 polymerase-inactive GDD mutation was included as a negative control.

Interferon-β inhibition of WT and mutant ZIKVs. Vero cells were seeded in 96-well plate (1.5×10⁴ cell per well) one day before interferon treatment and viral infection. The cells were infected at an MOI 0.05 in the presence of IFN-β (55, 167, 500, or 1,500 IU/ml). Viral infection and interferon treatment were initiated at the same time. At 48 h post-infection and interferon-β treatment, viral titers were quantified using the immunostaining focus assay on Vero cells.

### Results

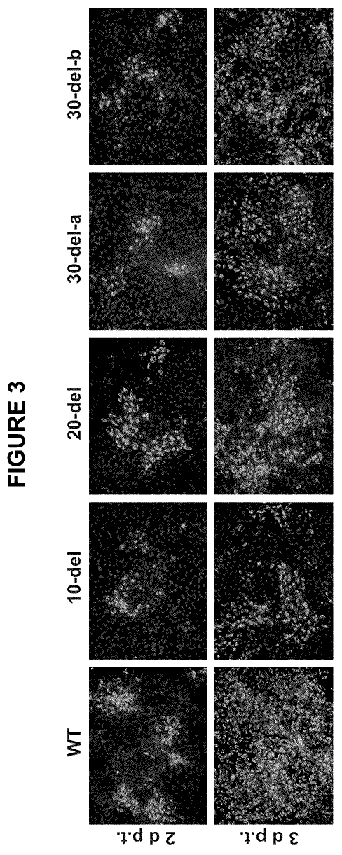

Upon transfection into Vero cells, all mutant genomic RNAs generated viral E protein-expression cells (FIG. 3) and infectious viruses (defined as P0 viruses). Compared with the WT, all mutants exhibited smaller infectious foci (FIG. 1B), slower replication kinetics, and lower peak titers (FIG. 1C). To examine the mutational effects on viral replication, we engineered the deletions into a luciferase ZIKV replicon (Reference 11). The replicon results showed that the 3′UTR deletions did not affect viral RNA translation (indicated by the luciferase signals at 2-6 h post-transfection), but decreased RNA synthesis (indicated by the luciferase activities at 24-48 h post-transfection; FIGS. 1D & 1E); a similar observation was previously reported for West Nile virus, a closely related flavivirus (Reference 12). Since the 3′UTR of flavivirus may also modulate host innate immune response (References 13, 14), we compared the susceptibility of the WT and mutant viruses to interferon inhibition. All four mutant viruses were much more sensitive to interferon-β inhibition than the WT virus, among which mutant 10-del exhibited the greatest inhibition (FIG. 1F). Collectively, these results indicate that 3′UTR deletions attenuate ZIKV replication through diminished viral RNA synthesis and increased vulnerability to type-I interferon inhibition.

### Example 3: Stability of Mutant Viruses

### Materials and Methods

Stability of 3′UTR mutants, RNA extraction, and RT-PCR. To examine the stability of 3′UTR mutants, we passaged them on Vero cells for five rounds (5 days for each round of culture),. Briefly, 1.5×10⁶ Vero cells were seeded into T-25 flask. The virus derived from RNA transfection, defined as P0 was used to infect the Vero cells. At 5 d p.i., culture fluid (100 μl) was transferred to a new T-25 flask containing Vero cells in 5 ml of culture medium. After five rounds of such passaging (P5), viral RNAs were extracted from the P5 culture fluids using QIAamp Viral RNA Kit (Qiagen). Viral RNAs were amplified by RT-PCR using SuperScript III one-step RT-PCR kits (Invitrogen). The P5 viruses were subjected to complete genome-length sequencing. Three independent passages were performed for each mutant virus.

Immunostaining focus assay. WT and P5 mutant viruses were analyzed using an immunostaining focus assay on Vero cells. For each mutant virus, three independent selections were performed on Vero cells.

Replication kinetics. Subconfluent Vero cells in 24-well plates (2×10⁵ cells per well) were infected with WT and P5 mutant viruses at a multiplicity of infection (MOI) of 0.01 in triplicate wells. Virus stocks were diluted in DMEM containing 2% FBS and 1% penicillin/streptomycin. One hundred microliters of virus were added to each well of the 12-well plates. After 1 h attachment (5% CO₂ at 37° C.), the inocula were removed, monolayers were washed three times with PBS, and 1 ml DMEM medium containing 2% FBS and 1% penicillin/streptomycin was added to each well. Culture fluids were quantified for infectious viruses on days 1 to 5 using the immunostaining focus assay on Vero cells.

### Results

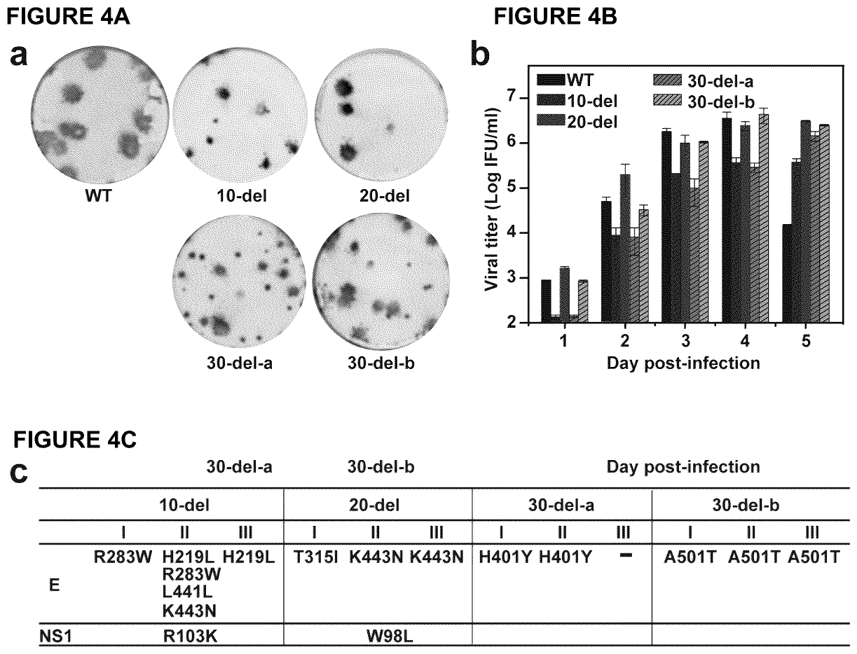

To test the stability of the mutant viruses, we passaged them five times on Vero cells (an approved cell line for vaccine production, see Reference 15). The passage 5 (P5) viruses developed larger infectious foci (FIG. 4A) and faster replication kinetics than the corresponding P0 viruses on Vero cells (Compare FIG. 4B with FIG. 1C). Complete genome sequencing of P0 and P5 viruses showed that all mutants retained the original deletions, but the P5 viruses had accumulated additional mutations in the E and/or NS1 genes, which presumably were Vero cell-adaptive mutation(s) and/or compensatory mutation(s) to 3′UTR deletions (FIG. 4C). In some embodiments, these mutations may be introduced into the infectious cDNA clone for vaccine production. Either way, the results indicated that the engineered deletions are stable when propagated on Vero cells, and further passaging of the mutant viruses on Vero cells to P20 did not change the engineered 3′UTR deletions.

### Example 4: Characterization of 3′UTR Mutants in the A129 Mouse Model

### Materials and Methods

Vaccination and challenge of mice. Three-week old A129 mice (n=8) were immunized by the subcutaneous (S.C.) route with 1×10⁴ IFU WT and mutant viruses. Mock-infected mice were given PBS by the same route. Mice were weighed and monitored daily for progression of disease. Mice were anesthetized and bled via the retro orbital sinus (R.O.) every two days. Viremias were quantified by an immunostaining focus assay from day 2 to 4 post-infection. On day 28 post-immunization, mice were anesthetized and bled to measure neutralization antibody titers using a mCherry ZIKV infection assay. The vaccinated mice were then challenged via the intraperitoneal (I.P.) route with 1×10⁵ PFU parental virus (ZIKV strain FSS13025). On day 2 post-challenge, the mice were bled to measure viremia. Blood was clarified post collection by centrifugation at 3,380×g for 5 min and immediately stored at −80° C. for storage. Viral titers of sera and inoculum were determined by an immunostaining focus assay on Vero cells, as described above. All animal testing was performed in accordance with UTMB policy as approved by the UTMB IACUC.

Construction of mCherry ZIKV. A DNA fragment encoding the first 25 amino acids of C gene, the mCherry gene, and the foot-and-mouth virus 2A protein was in-frame fused with the open-reading-frame of ZIKV genome. The expression of mCherry in transfected Vero cells was analyzed by a fluorescent microscopy at days 2-6 post-transfection. The mCherry ZIKV was used to estimate antibody neutralization titers of mouse sera.

Antibody neutralization assay. Neutralizing activity of mouse sera was assessed using a newly established mCherry ZIKV. The sera were 2-fold serially diluted starting at 1:100 in DMEM with 2% FBS and 1% penicillin/streptomycin. Serial dilution of mice sera was incubated with mCherry ZIKV at 37° C. for 2 h. Antibody-virus complexes were added to pre-seeded Vero cells in 96-well plates. After 48 h post-infection, cells were visualized by fluorescence microscopy using Cytation 5 Cell Imaging Multi-Mode Reader (Biotek) to quantify the mCherry fluorescence-positive cells. The percentage of fluorescence-positive cells in the non-treatment controls was set at 100%. The fluorescence-positive cells from serum-treated wells were normalized to those of non-treatment controls. A four-parameter sigmoidal (logistic) model in the software GraphPad Prism 7 was used to calculate the neutralization titers (NT₅₀).

### Results

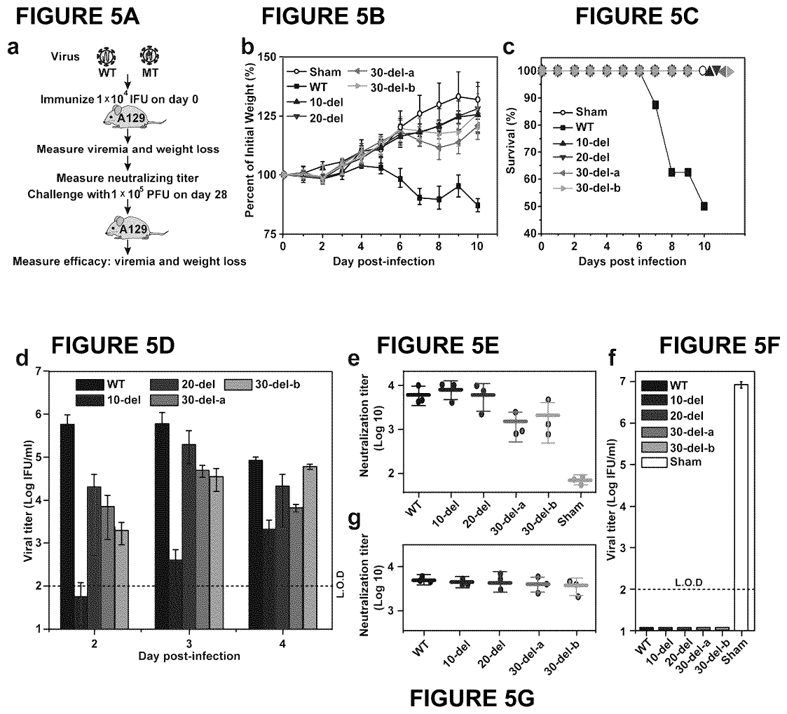

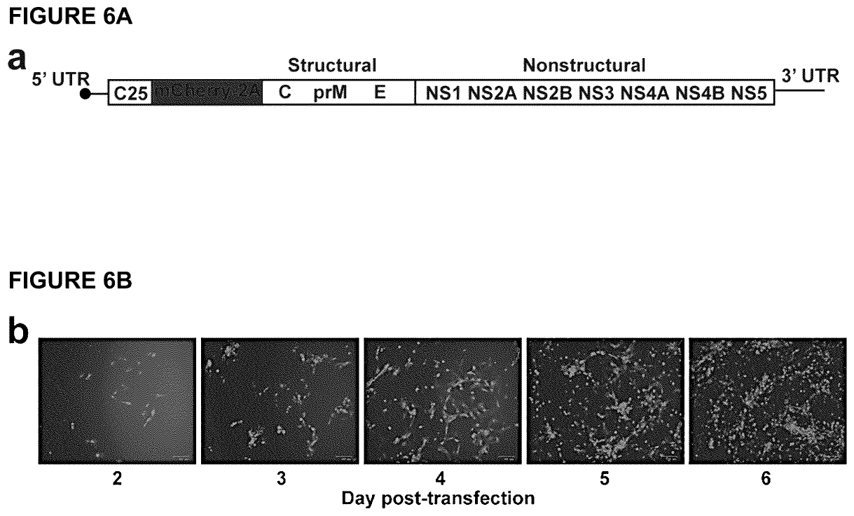

We evaluated the immunogenicity and efficacy of the mutant viruses in an A129 (interferon α/β receptor-deficient) mouse model (Reference 16) (FIG. 5A). After subcutaneous (S.C.) inoculation with 1×10⁴ IFU of virus, mice infected with the WT virus had significantly more weight loss than those infected with mutant viruses; whereas the differences in mean weight loss among the four mutant virus-infected groups were not statistically significant (FIG. 5B). About 50% of the mice succumbed to the WT virus infection, whereas no mortality was observed in the mutant virus-infected mice (FIG. 5C). The WT virus produced significantly higher peak viremia than the mutant viruses, among which the 10-del virus had the lowest viremic profile (FIG. 5D). The viremia for 10-del mutant dropped to 700 IFU, 350 IFU, and undetectable on days 5, 6, and 7 post-infection, respectively. Sequencing analysis confirmed that the engineered deletions were retained without other mutations in the mutant viruses recovered from the mouse sera. On day 28 post-infection, mouse sera were taken and quantified for pre-challenge neutralization titers using an mCherry ZIKV (FIG. 6). Comparable pre-challenge neutralization titers of (1.8±1.1)×10³ to (8.6±1.5)×10³ were observed among the WT and mutant virus-infected mice (FIG. 5E). After challenge with 1×10⁵ IFU of WT ZIKV (Cambodian strain FSS13025) on day 28 post-immunization, the immunized mice had no detectable peripheral viremia, whereas the mock-immunized group produced a mean viremia of (8.5±1.5)×10⁶ IFU/ml on day 2 post-challenge (FIG. 5F). On day 28 post-challenge, we measured the neutralization titers of the mouse sera again; remarkably, the post-challenge neutralization titers were equivalent to the pre-challenge neutralization titers (compare FIGS. 5E & 5G), suggesting that a sterilizing antibody response had been achieved by a single vaccination. Altogether, the results demonstrate that the mutant viruses are highly attenuated, immunogenic, and protective in A129 mice.

### Example 5: Further Characterization of 10-del Mutant ZIKV

### Materials and Methods

Viruses. The Puerto Rico strain PRVABC59 (GenBank number KU501215) was obtained from WRCEVA. All the cell lines are tested negative for mycoplasma.

Immunization with 100 IFU 10-del virus. Three-week-old A129 mice (n=5) were immunized with 100 IFU WT or 10-del virus via the S.C. route. Viremia was quantified by immunostaining focus assay from day 2 to 6. On day 28 post-immunization, mouse sera were quantified for ZIKV neutralizing antibody titers. Also on day 28 post-immunization, the mice were challenged with 1×10⁶ IFU of ZIKV (Puerto Rico strain PRVABC59) via the I.P. route. Viremias were quantified by immunostaining focus assay on day 2 post-challenge.

Immunization with 10 IFU 10-del virus. Three-week-old A129 mice (n=2 for each virus) were immunized with 10 IFU WT or 10-del ZIKV via the S.C. route. On day 28 post-immunization, mouse sera were quantified for ZIKV neutralizing antibody titers. On the same day, the mice were challenged with 1×10⁶ IFU of ZIKV (Puerto Rico strain PRVABC59) via the I.P. route. On day 2 post-challenge, viremias were quantified using an immunostaining focus assay.

### Results

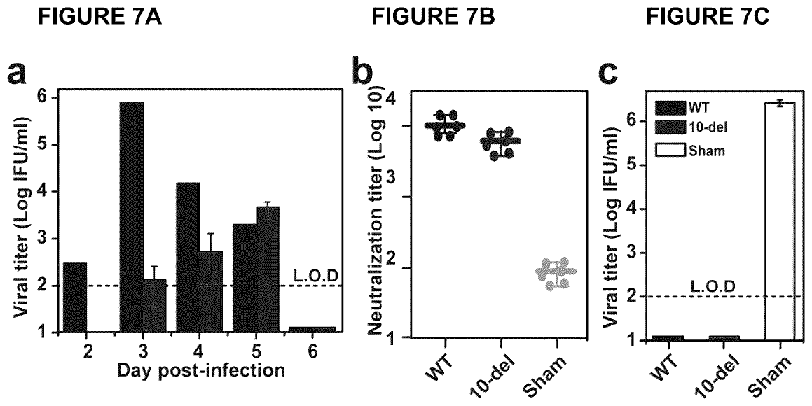

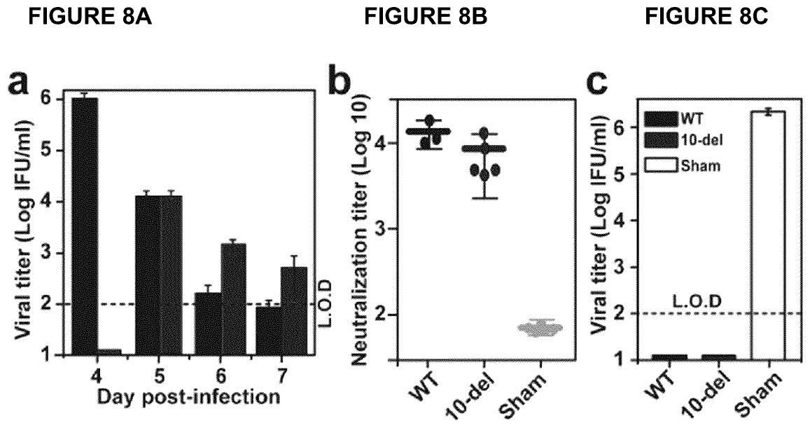

Since the 10-del virus produced the lowest viremia in mice (FIG. 5D), yet induced a neutralizing antibody response comparable to those of the WT and other mutants (FIG. 5E-5G), we prioritized this mutant for further characterization. At a dose of 100 IFU, 10-del virus-infected mice showed a delayed peak viremia that was >100-fold lower than that of the WT virus (FIG. 7A). Equivalent levels of pre-challenge neutralization titers were induced by the WT and 10-del viruses (FIG. 7B), leading to complete protection from viremia after challenge with 1×10⁶ IFU of Puerto Rico ZIKV strain PRVABC59 (FIG. 7C). Furthermore, even when immunized at a dose of only 10 IFU, 10-del virus-infected mice generated a neutralization titer of (9.7+6.8)×10³, and were fully protected from viremia after challenge (FIG. 8).

It is noteworthy that mice immunized with different doses of 10-del mutant (10, 10², and 10⁴ IFU) induced similar neutralization antibody titers and completely prevented viremia upon challenge (compare FIG. 5 and FIGS. 7 & 8).

Collectively, these results demonstrate that the 10-del virus is a potent vaccine candidate.

### Example 6: Comparison of P0 and P5 10-del Viruses

### Materials and Methods

Immunization with P0 or P5 10-del virus: Three-week-old A129 mice (n=5) were immunized with 100 IFU P0 or P5 10-del virus via the S.C. route. Viremia were quantified by immunostaining focus assay from day 4 to 6.

Pre-challenge neutralization antibody titers: On day 28 post-immunization, mouse sera were quantified for ZIKV neutralizing antibody titers.

Viremia after challenge with wild-type ZIKV: On day 28 post-immunization, the mice were challenged with 1×10⁶ IFU of an epidemic strain of ZIKV (Puerto Rico strain PRVABC59) via the I.P. route. On day 2 post-challenge, viremias were quantified using an immunostaining focus assay.

### Results

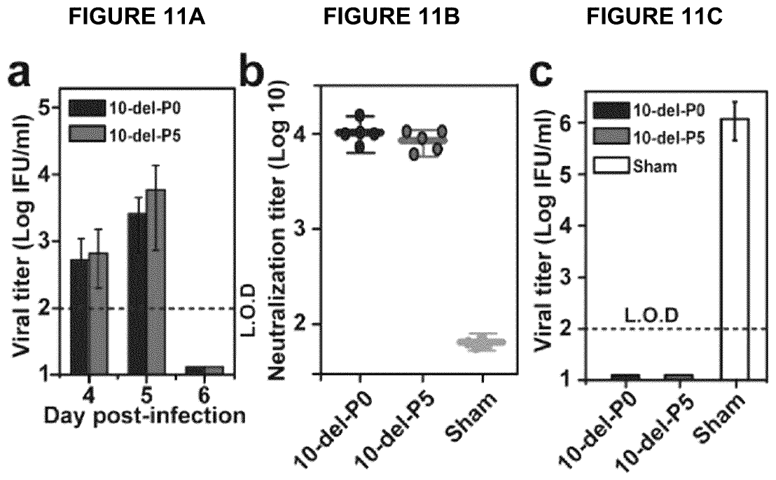

Since P5 virus accumulated Vero cell-adaptive mutations (FIG. 4C), we compared the virulence and immunogenicity between the P0 and P5 10-del viruses in the A129 mice. After immunization with 100 IFU virus via the S.C. route, the P0 and P5 viruses generated comparable viremia and induced equivalent neutralization titers (FIGS. 11A & 11B). After challenging with 1×10⁶ IFU of Puerto Rico strain PRVABC59 ZIKV via the I.P. route, no viremia was detected in the P0 or P5 virus-vaccinated mice; in contrast, robust viremia were detected in the sham group (FIG. 11C). These results indicate that the Vero cell-adaptive mutations recovered from the P5 virus do not significantly affect the virulence and immunogenicity of the 10-del virus.

### Example 7: T Cell Responses in A129 Mice Immunized With 10-del ZIKV

### Materials and Methods

Measuring T cell responses in A129 mice. A129 mice were infected with 1×10⁴ IFU WT and 10-del viruses. On day 28 post-infection, mouse spleens were harvested. Splenocytes were counted, cultured ex vivo with WT ZIKV for 24 h, and stained for markers (IFN-γ, CD3, and CD4 or CD8). The T cells were gated based on staining for these markers, percentages of CD4⁺IFN-γ⁺ cells and CD8⁺IFN-γ⁺ cells were counted, and average total number of T cell subsets per spleen was recorded. Supernatants from the ex vivo culture were harvested on day 2 after WT ZIKV re-stimulation, and measured for IFN-γ and IL-2 production.

Bio-Plex immuneassay. Approximately 3×10⁵ splenocytes were plated in 96-well plates and stimulated with 1.25×10⁴ IFU ZIKV strain FSS13025 for 48 h. Culture supernatants were harvested and cytokine production were measured using a Bio-Plex Pro Mouse Cytokine Assay (Bio-Rad, Hercules, CA).

Intracellular cytokine staining (ICS). Approximately 2.5×10⁶ splenocytes were stimulated with 1×10⁵ IFU live ZIKV (strain FSS13025) for 24 h. During the final 5 h of stimulation, BD GolgiPlug (BD Bioscience) was added to block protein transport. Cells were stained with antibodies for CD3, CD4, or CD8; fixed in 2% paraformaldehyde, and permeabilized with 0.5% saponin before addition of anti-IFN-γ, or control rat IgG1 (e-Biosciences). Samples were processed with a C6 Flow Cytometer instrument. Dead cells were excluded on the basis of forward and side light scatter. Data were analyzed with a CFlow Plus Flow Cytometer (BD Biosciences).

### Results

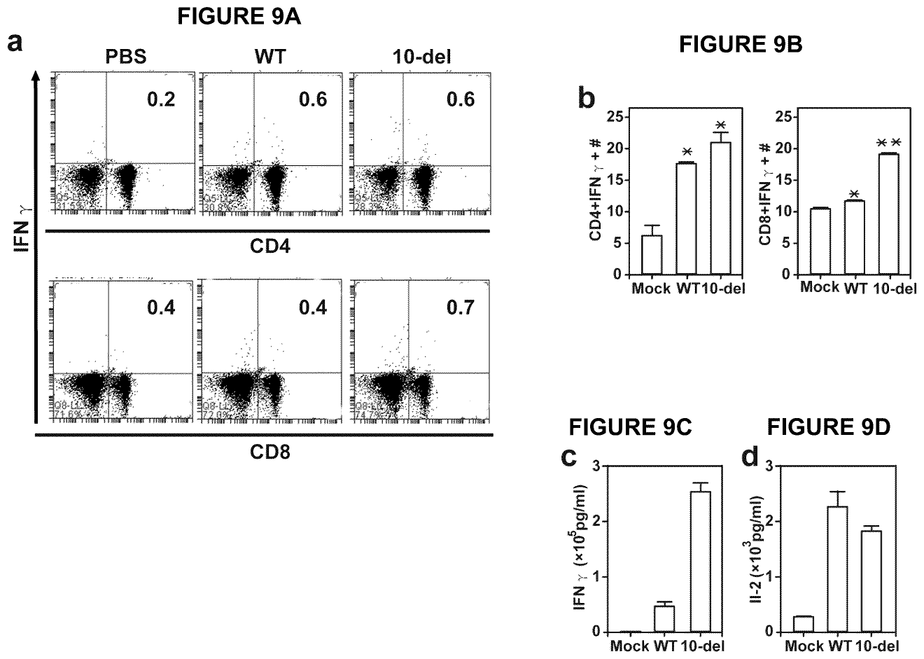

We analyzed the T cell responses in A129 mice immunized with 1×10⁴ IFU of WT and 10-del viruses. On day 28 post-immunization, ZIKV-specific T cells were re-stimulated with live WT virus in vitro, and analyzed using an intracellular cytokine staining (ICS) assay and a Bio-Plex immunoassay. The results showed that both WT and mutant virus-immune CD4⁺ and CD8⁺ T cells had higher IFN-γ responses than the mock-immunized group (FIGS. 9A & 9B). Furthermore, these immune T cells induced more IFN-γ (FIG. 9C) and IL2 (FIG. 9D) than the mock group; particularly, the 10-del mutant-immune T cells produced 4-fold higher IFN-γ than the WT virus-immune group. These results indicate that 10-del vaccine candidate induces a robust T cell response.

### Example 8: Safety of 10-del Vaccine Candidate

### Materials and Methods

Organ virus titers. The heart, lung, liver, spleen, kidney, muscle, brain, testis, and eye were harvested on day 6 and 10 post-infection after 1×10⁴ IFU of WT and 10-del virus vaccination via the S.C. route. Organ titrations were performed using a immunostaining focus assay as described above. In brief, 500 μl of DMEM with 2% FBS and penicillin/streptomycin along with a steel ball bearing were placed in a 2-ml Eppendorf tube. The organ (whole or part) was placed in the tube. Tubes were weighed, and organ weight was determined by subtracting the tube weight. Tissues were homogenized in a Qiagen TissueLyser II shaking at 26 p/second for 5 minutes. The homogenate was clarified by centrifugation for 5 min at 12,000 rpm and titrated on Vero monolayer using an immunostaining focus assay. The titer was then adjusted for volume and organ weight to report the organ loads as IFU/g (Reference 16).

Neurovirulence on newborn CD1 mice. Groups of 1-day-old outbred CD1 mice (n=7−10) were injected intracranially (I.C.) with WT or 10-del with serial tenfold dilutions from 10,000 IFU to 10 IFU. Mice were monitored daily for morbidity and mortality.

Experimental infection of mosquitoes with ZIKV. Aedes aegypti mosquitoes derived from a Galveston, Texas colony were exposed for 45 min to blood-meals consisting of 1% (weight/volume) sucrose, 20% (vol/vol) FBS, 5 mM ATP, 33% (vol/vol) PBS-washed human blood cells (UTMB Blood Bank), and 33% (vol/vol) DMEM medium and combined with 1 ml virus offered in Hemotek 2-ml heated reservoirs (Discovery Workshops) covered with a mouse skin. Virus titers in the blood meals were 1×10⁶ IFU/ml. Infectious blood meals were loaded on cartons containing Ae. aegypti. Fully engorged mosquitoes were incubated at 28° C., 80% relative humidity on a 12:12 h light: dark cycle with ad lib access to 10% sucrose solution for 7 days and harvested by freezing at −20° C. for 3 h. Whole mosquitoes were individually homogenized (Retsch MM300 homogenizer, Retsch Inc., Newton, PA) in DMEM with 20% FBS and 250 μg/ml amphotericin B and stored at −80° C. Samples were centrifuged for 10 min at 5,000 rpm, and 75 μl of each sample supernatant were inoculated into 96-well plates containing Vero cells at 37° C. and 5% CO₂ for 3 days, when they were fixed with a mixture of ice-cold acetone and methanol (1:1) solution and immunostained as described above. Infection was determined by detection of virus in the homogenized mosquito. The infection rate was recorded as the fraction of positive mosquitoes divided by the total number of engorged, incubated mosquitoes.

Testis and sperm count analyses: A129 mice were infected with 1×10⁴ IFU of WT or 10-del mutant virus. A mock-infected group with PBS was included as a negative control. On day 16 p.i., the mice were euthanized and necropsied; epididymis and testes were harvested immediately as previously described (Reference 32). Briefly, the epididymis was placed into 1 ml of pre-warmed M2 media at 37° C. To release the sperm, the epididymis was cut lengthwise six times and incubated for 10 min, agitating every 2 min at 37° C. Following the incubation, the media containing the sperm was immediately diluted 1:50 into pre-warmed M2 media and counted on a hemocytometer. Motile sperms were categorized into progressive and non-progressive. Progressively motile sperms are described as continuous displacement of the head by flagellar movement. Non-progressively motile sperms are described as little to no displacement of the head by flagellar movement. In the non-motile sperms, no flagellar movement was observed.

### Results

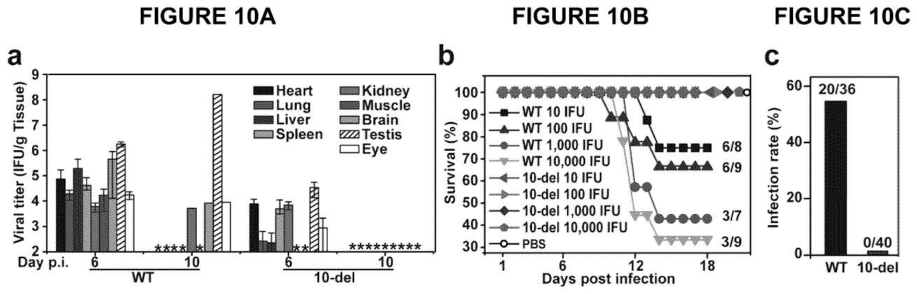

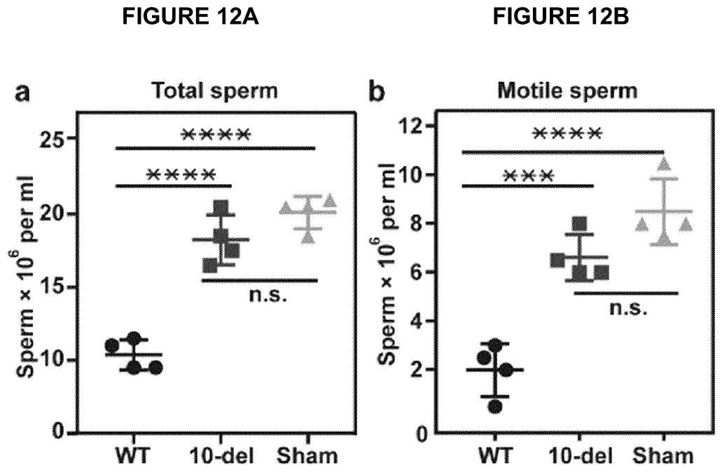

Three sets of experiments were performed to analyze the safety of the 10-del vaccine candidate. First, we measured the viral loads in different organs after S.C. inoculation of A129 mice with 1×10⁴ IFU of WT or 10-del viruses (FIG. 10A). On day 6 post-infection, the WT-infected mice had high viral loads in all organs tested, whereas the 10-del-infected mice had no virus in muscle or brain, and lower viral loads in heart, lung, liver, spleen, kidney, testes, and eye. On day 10 post-infection, WT virus-infected mice retained viral loads in kidney, brain, testis, and eye, among which testes had the highest mean titer; in contrast, no virus could be detected (<10² IFU/mL) in any organs from the 10-del-infected mice. Since ZIKV infection was reported to damage the testes in mice (References 17, 18), we examined the effect of immunization on the function of testes in the A129 mice. On day 16 post-immunization, similar weight and size of testes were recovered from the mock-, WT virus-, and 10-del mutant-infected mice. However, motile and total sperm counts were reduced in the WT virus-infected mice, whereas the 10-del virus-infected mice did not significantly compromise the sperm counts when compared with the mock group (FIG. 12).

Second, we examined the neurovirulence of 10-del virus through intracranial (I.C.) injection of one-day-old CD1 mice (FIG. 10B). The newborn mice succumbed to WT virus infection in a dose-responsive manner; even a dose of 10 IFU resulted in 25% mortality. Remarkably, mice infected with 10-del virus did not show any apparent disease or death, even at a dose of 1×10⁴ IFU. Finally, we determined if 10-del virus could infect Aedes aegypti mosquitoes, the main transmission vector of ZIKV in the Americas (References 19, 20). After exposure to artificial blood-meals containing 1×10⁶ IFU/ml of WT or 10-del virus and incubation for 7 days, 56% of the engorged mosquitoes were infected by the WT virus, whereas no mosquitoes were infected by the 10-del mutant. Furthermore, intrathorax injection of 10-del virus to mosquitoes did not yield any infectious virus on day 7 after injection. Collectively, our results demonstrated that the 10-del virus significantly reduced or eliminated viral loads in mouse organs, decreased neurovirulence by >1,000-fold, and attenuated its ability to infect the principal urban mosquito vector, all suggestive of an excellent safety profile.

Our data indicate that the 3′UTR 10-del ZIKV is a promising live-attenuated vaccine candidate with a good balance between immunogenicity and safety. A single immunization elicited robust antibody and T cell responses, and significantly, unlike the subunit and inactivated ZIKV vaccines published to date, likely induces sterilizing immunity and providing complete protection against parental and epidemic strains of ZIKV. Vaccine-induced sterilizing immunity is likely critical for a successful ZIKV vaccine to prevent viremia and congenital abnormalities. The safety profile of this vaccine candidate is highlighted by the low viremia, little and transient viral loads in organs, and limited weight loss in the severe A129 mouse model, as well as a complete lack of morbidity and mortality in one-day-old mice after receiving an intracranial (I.C.) injection. The latter safety result is impressive because I.C. inoculation with YFV 17D and JEV SA14-14-2 (two licensed live-attenuated flavivirus vaccines) results in lethal disease in one-day-old newborn mice (References 21, 22). Although potential homologous recombination between the WT and vaccine ZIKVs might pose a safety liability for the 10-del vaccine candidate, it should be noted that recombination events are rare and could not be detected in cell culture (References 23-27). Compared with the chimeric, live-attenuated ZIKV vaccine (e.g., YFV 17D expressing ZIKV prM-E or DENV expressing ZIKV prM-E (Reference 28)), our 3′UTR mutant vaccine has the advantage of retaining all ZIKV structural and nonstructural genes that may contribute to antiviral protection, as indicated from dengue vaccine studies (References 29, 30).

Compared with the chimeric, live-attenuated ZIKV vaccine (e.g., YFV 17D expressing ZIKV prM-E), our 3′UTR mutant vaccine has the advantage of retaining all ZIKV structural and nonstructural genes that may contribute to antiviral protection, as indicated from dengue vaccine studies (References 29, 30). Mechanistically, the 3′UTR mutant viruses appeared to be attenuated through decreased viral RNA replication and increased sensitivity to type-I interferon inhibition. The latter mechanism is in agreement with a recent report that genetic diversity at the 3′UTR of DENV contributes to epidemic potential (Reference 14). Taken together, our results indicate that the 3′UTR mutant ZIKV is an attractive vaccine candidate that should be advanced to non-human primates for further development.

Additional in vivo experiments to characterize safety and efficacy of live attenuated ZIKV strains with 3′UTR deletions

### Materials and Methods

Additional in vivo systems using the Materials and Methods and the examples which follow were also employed to characterize the safety and efficacy of vaccine candidates, including C57BL/6J mouse pregnancy, A129 mouse testis protection, viral loading in A129 mouse organ, CD-1 mouse neurovirulence, and rhesus macaque efficacy (see details below). The protocols for each of these experimental systems have been previously established (e.g., inoculum dose, infection route, challenge dose, and end-point measurement), and were not altered when evaluating these vaccine candidates. Therefore, different inoculum doses, challenge doses, and end-point measurements (e.g., qRT-PCR to measure viral RNA and focus forming assay to measure infection virus) were used in different in vivo systems according to the established protocols. The detailed information is described below and indicated in the Examples which follow.

Mouse Studies: All in vivo experiments using mice were performed in accordance with the recommendations in the Guide for the Care and Use of Laboratory Animals of the National Institutes of Health. The protocols were approved by the Institutional Animal Care and Use Committee (IACUC) at the Washington University School of Medicine (Assurance Number A3381-01) and the IACUC at the University of Texas Medical Branch (UTMB; Protocol Number 0209068B). Dissections and footpad injections were performed under anesthesia that was induced and maintained with ketamine hydrochloride and xylazine at the Washington University or isoflurane at UTMB. All efforts were made to minimize animal suffering. Rhesus macaque experiments were reviewed and approved by Vaccine Research Center Animal Care and Use Committee at the National Institute of Allergy and Infectious Diseases, the National Institutes of Health. The non-human primate experiments were performed in compliance with the pertinent regulations and policies from the National Institutes of Health.

Viruses and cells: The ZIKV Cambodian strain FSS13025 (GenBank number KU955593.1) was produced from an infectious cDNA clone (Reference 35). The ZIKV-3′UTR-Δ10-LAV and ZIKV-3′UTR-Δ20-LAV strains were generated as described. The Zika Puerto Rico strain PRVABC59 (GenBank number KU501215) and Dakar 41519 strain (GenBank number HQ234501.1) were obtained originally from Dr. Robert Tesh from the World Reference Center of Emerging Virus and Arboviruses (WRCEVA) at UTMB. The mouse-adapted ZIKV-Dakar 41519 strain was passaged twice in Rag1−/−mice (Jackson Laboratories) and described previously (Reference 29). Vero cells were purchased from the American Type Culture Collection (ATCC CCL-81; Bethesda, MD), and maintained at 37° C. with 5% CO₂ in a high glucose Dulbecco modified Eagle medium (DMEM; Invitrogen, Carlsbad, CA) with 10% fetal bovine serum (FBS; HyClone Laboratories, Logan, UT) and 1% penicillin/streptomycin (Invitrogen, Carlsbad, CA). All cell lines tested negative for mycoplasma.

Antibodies: The following antibodies were used in this study: anti-mouse IFN alpha/beta receptor 1 (Ifnar1) monoclonal antibody (clone MAR1-5A3; Leinco Technologies, Inc., St. Louis, Missouri); ZIKV-specific HMAF (hyper-immune ascites fluids; obtained from WRCEVA), anti-mouse IgG antibody labeled with horseradish peroxidase (KPL, Gaithersburg, MD), and goat anti-mouse IgG conjugated with Alexa Fluor 488 (Thermo Fisher Scientific, Providence, RI).

A129 mouse experiments: A129 mice were bred in the animal facilities at UTMB. All mice were housed in pathogen-free mouse facilities. Three-week or 15-week old male A129 mice were infected with PBS (two sham groups), 10⁴ FFU of ZIKV-3′UTR-Δ10-LAV (Δ10), or 10³ FFU of ZIKV-3′UTR-Δ20-LAV (Δ20). Mice were anesthetized and bled via the retro orbital sinus (R.O.) for viremia testing. At day 28 post-immunization, mice were measured for neutralizing antibody titers using an mCherry ZIKV infection assay (Reference 36). On the same day, one sham group of PBS-immunized mice and Δ10- and Δ20-immunized mice were challenged with 10⁶ FFU of ZIKV PRVABC59. Another sham group of mice was used as an unchallenged negative control. At day 49 post-immunization, mice were euthanized and necropsied. Epididymis and testes were harvested immediately as previously described (Reference 32). Motile and non-motile sperms were counted manually on an emocytometer by microscopy. Total sperm counts equal to the sum of motile and non-motile sperms. For quantification of viral loads, testes were homogenized and infectious viral levels were measured by a focus forming assay or quantitative reverse transcriptase PCR (qRT-PCR) (Reference 36).

The qRT-PCT primer/probe set includes forward primer (1193F: 5′-CCGCTGCCCAACACAAG-3′) (SEQ ID NO:10), reverse primer (1269R: 5′-CCACTAACGTTCTTTTGCAGACAT-3′) (SEQ ID NO:11), and probe (5′-FAM/AGCCTACCT/ZEN/TGACAAGCAATCAGACACTCAA/3IABkFQ-3′)(SEQ ID NO:12). The probe contains a 5′-FAM reporter dye, 3′ IBFQ quencher, and an internal ZEN quencher.

Mouse pregnancy experiments: C57BL/6J mice were bred and housed in pathogen-free mouse facilities at Washington University School of Medicine. One day prior to immunization, the eight-week old female C57BL/6J mice were dosed with 0.5 mg of anti-Ifnar1 antibody via an intraperitoneal route. Subsequently, mice were subcutaneously inoculated in the footpad with 10⁵ FFU of ZIKV-3′UTR-Δ10-LAV or PBS sham. Immunized wild-type (WT) C57BL/6 female mice were mated with naïve WT male mice. At embryonic day 5 (E5), pregnant dams were injected intraperitoneally with 2 mg of anti-Ifnar1 antibody. On E6, mice were inoculated subcutaneously with 10⁵ FFU of mouse-adapted ZIKV Dakar 41519 via footpad injection. All animals were sacrificed on E13 and analyzed for viral loads in placentas, fetuses, and maternal tissues. Briefly, maternal blood, organs from dams (brain and spleen) and fetuses (placenta and fetal head) were collected. Serum was prepared after coagulation and centrifugation. Organs were weighed and homogenized using a bead-beater apparatus (MagNA Lyser, Roche). Viral RNA was extracted from serum and tissue samples using the RNeasy Mini kit (Qiagen). The viral RNA levels were determined by TaqMan one-step qRT-PCR on an ABI 7500 Fast Instrument using standard cycling conditions. Viral burden is expressed on a log10 scale as viral RNA equivalents per gram or per milliliter after comparison with a standard curve produced using serial 5-fold dilutions of ZIKV RNA from known quantities of infectious virus. The following primer/probe set was used for ZIKV qRT-PCR: forward primer (1183F: 5′-CCACCAATGTTCTCTTGCAGACATATTG-3′)(SEQ ID NO:13), reverse primer (1268R: 5′-TTCGGACAGCCGTTGTCCAACACAAG-3′)(SEQ ID NO:14), and probe (1213F: 5′-56-FAM/AGCCTACCT TGACAAGCAGTC/3IABkFQ-3′) (SEQ ID NO:15). Wherever indicated, viral burden for some samples was determined by focus forming assay on Vero cells (Reference 28).

Quantification of viral load in organs from A129 mice: A129 mice were infected and organs were quantified for viral load using a focus forming assay (Reference 28). Viral RNA in testis also was quantified by qRT-PCR. Briefly, testes were harvested and placed in DMEM with beads for homogenization. After homogenization, the supernatant was used to extract viral RNA using RNeasy Mini kit (Qiagen). Extracted RNA was eluted in 40 μl RNase-free water. qRT-PCR assays were performed on the LightCycler® 480 System (Roche) following the manufacturer's protocol by using a 50-μl reaction of the QuantiTect Probe RT-PCR Kit (QIAGEN) and 10 μl RNA template. The viral load was calculated based on a standard curve produced using serial 10-fold dilutions of ZIKV RNA from known quantities of infectious virus. The qRT-PCT primer/probe set includes forward primer (1193F: 5′-CCGCTGCCCAACACAAG-3′)(SEQ ID NO:16), reverse primer (1269R: 5′-CCACTAACGTTCTTTTGCAGACAT-3′)(SEQ ID NO:17), and probe (5′-FAM/AGCCTACCT/ZEN/TGACAAGCAATCAGACACTCAA/3IABkFQ-3′) (SEQ ID NO:18). The probe contains a 5′-FAM reporter dye, 3′ IBFQ quencher, and an internal ZEN quencher.

Vaccination of non-human primates: Rhesus macaque (Macaca mulatta) experiments were performed at Bioqual, Inc. (Rockville, MD). All animal experiments were reviewed and approved by the Animal Care and Use Committee of the Vaccine Research Center, the National Institute of Allergy and Infectious Diseases, the National Institutes of Health. Animals were housed and cared in accordance with local, state, federal, and institutional policies in an American Association for Accreditation of Laboratory Animal Care-accredited facility at the Bioqual Inc. Rhesus macaques (3-4/group) were randomized by body weight, gender, and age, and subcutaneously administered with 10³ FFU of parental WT ZIKV strain FSS13025, ZIKV-3′UTR-Δ10-LAV, ZIKV-3′UTR-Δ20-LAV, or PBS sham at day 0. Blood was collected at day 2, 3, 4, 5, 7, and 10 for viremia testing and weekly for analysis of antibody responses by an mCherry ZIKV neutralization assay (References 36, 38). The immunized animals were subcutaneously challenged with 10³ FFU of ZIKV strain PRVABC59 at week 8. Blood samples were collected for determination of viral load at day 2, 3, 4, 5, 7, and 10 and neutralization antibody at week 2, 4, and 6 post-challenge.

Viremia from rhesus macaques was quantified by a qRT-PCR assay and focus forming assay as described in the proceeding section. For qRT-PCR quantification, viral RNA was extracted from rhesus serum using QIAamp Viral RNA Kits (QIAGEN) following the manufacture instruction. Extracted RNA was eluted in 40 μl RNase-free water. qRT-PCR assay was performed as described above. In vitro transcribed full-length ZIKV RNA was used as a standard for qRT-PCR quantification. The primer/probe set is described in the proceeding section.

Neutralization assay and neurovirulence: All antibody neutralization titers were determined using an mCherry ZIKV as previously reported (Reference 36). The dilution folds that neutralized 50% of mCherry ZIKV infection (NT50) were presented. For measuring neurovirulence, 1-day-old outbred CD-1 mice (Charles River) were injected intracranially with indicated amounts of viruses. The infected mice were monitored for morbidity and mortality as reported previously (Reference 36).

Mosquito infection: For measuring mosquito infection, artificial blood-meal spiked with 10⁶ FFU/ml of indicated viruses was used to feed Aedes aegypti mosquitoes (derived from a Galveston, Texas), and engorged mosquitoes were incubated at 28° C., 80% relative humidity on a 12:12 h light: dark cycle with ad libitum access to 10% sucrose. The infection rates were determined at day 7 post-feeding as reported previously (Reference 10).

Data analysis: All data from these experiments the results of which are described in the examples which follow and are contained in the figures referenced therein were analyzed with GraphPad Prism v7.02 software. Data are expressed as the mean ±standard deviation (SD). Comparisons of groups were performed using Mann-Whitney test or one-way ANOVA with a multiple comparisons correction. A P value of <0.05 indicates statistically significant.

### Example 9: Prevention of Vertical Transmission in Pregnant Mice

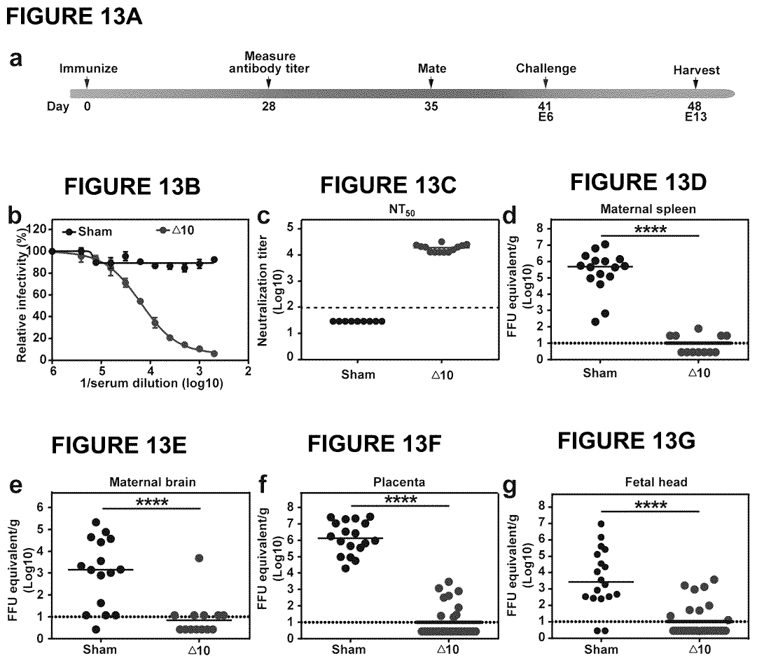

The live-attenuated vaccine candidate described herein which contains a 10-nucleotide deletion in the 3′UTR of ZIKV genome (ZIKV-3′UTR-Δ10-LAV) was tested for its ability to prevent in utero transmission. In these experiments we subcutaneously inoculated 10⁵ focus-forming units (FFU) of ZIKV-3′UTR-Δ10-LAV or PBS-sham into 8-week-old wild-type C57BL/6 female mice (FIG. 13A). Because mice are not a native host for ZIKV due in part to a species-dependent lack of antagonism of type I IFN signaling (References 39, 40), we administered 0.5 mg of anti-Ifnar1 blocking antibody to female mice one day prior to vaccination to facilitate transient replication of ZIKV-3′UTR-Δ10-LAV and to attempt to produce disease (Reference 41). At day 28 post-vaccination, the animals were phlebotomized and serum was analyzed for neutralizing antibody (FIG. 13B); all ZIKV-3′UTR-Δ10-LAV-immunized mice developed high neutralizing antibody titers of 18,900±5,900 (mean ±standard deviation; n=13), whereas, as expected, the PBS-immunized animals did not develop detectable neutralizing antibodies (FIG. 13C). At day 35 post-vaccination, the immunized females were mated with 12-week-old wild-type (WT) C57BL/6 male mice and monitored for vaginal plugs (FIG. 13A). At embryo day 6 (E6), pregnant mice were challenged subcutaneously with 10⁵ FFU of a mouse-adapted, pathogenic ZIKV African strain Dakar 41519 (Reference 42); to facilitate ZIKV-Dakar dissemination to the maternal decidua and fetal placenta, the pregnant mice were administered 2 mg of anti-Ifnar1 antibody at E5, one day before the challenge. At E13, maternal and fetal organs were harvested and measured for viral load.

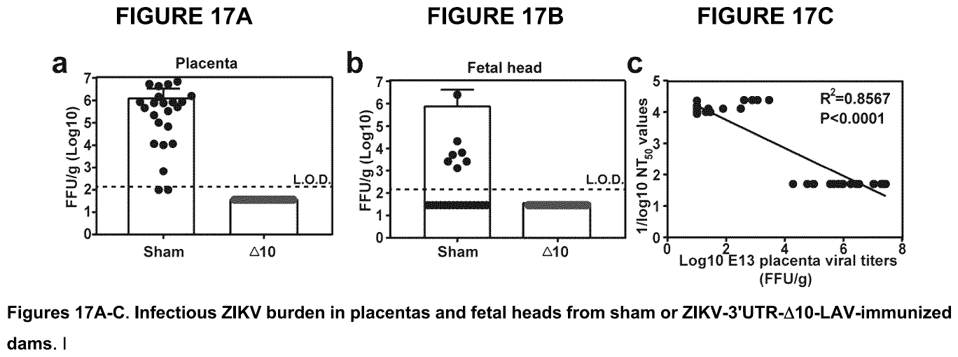

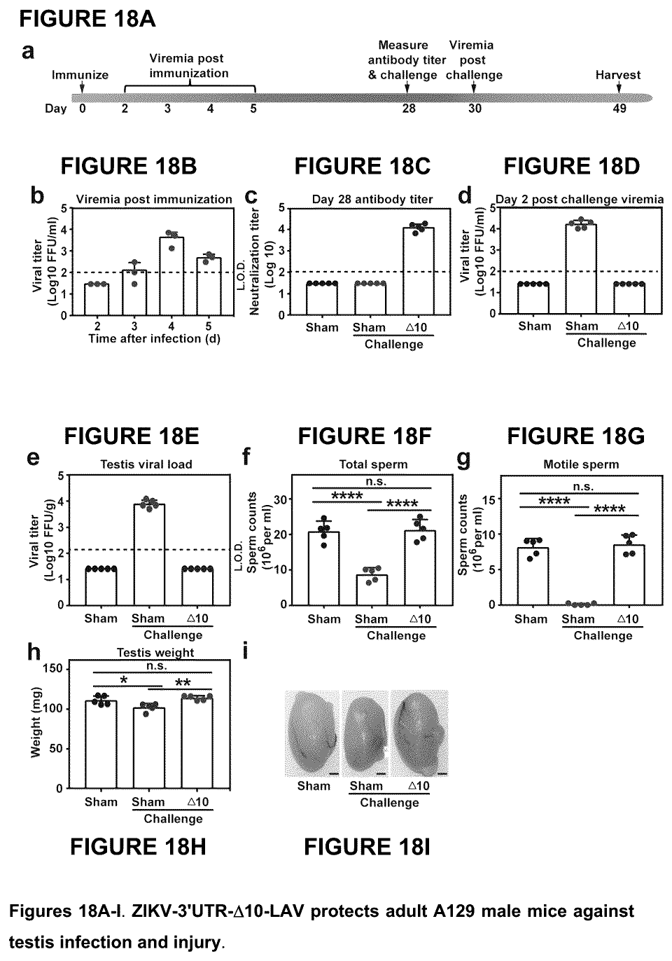

After a single immunization, ZIKV-3′UTR-Δ10-LAV reduced median viral loads in the maternal spleen and brain by ˜49,000-fold and ˜120-fold, respectively (FIG. 13D-E). Placentas and fetal heads from the vaccinated dams showed 138,000-fold and 260-fold decreases in viral RNA loads, respectively, when compared with the PBS-immunized dams (FIG. 13F-G). Notably, 21 of 30 (70%) placentas and 21 of 30 (70%) fetal heads from ZIKV-3′UTR-Δ10-LAV-immunized dams had viral RNA loads at or below the detection limit; no infectious virus was recovered by focus forming assay from the placentas or fetal heads from the vaccinated dams (FIG. 17A-B). In addition, the immune correlate between the EC₅₀ values and the levels of infectious viruses recovered in the placenta revealed an expected inverse relationship between neutralizing titers and levels of ZIKV particles in placenta (FIG. 17C). Collectively, the data suggest that ZIKV-3′UTR-Δ10-LAV protects maternal organs from infection, partially prevents viral transmission to the fetus during an early stage of pregnancy, and limits fetal replication.

### Example 10: Protection of ZIKV-induced Damages to Testes

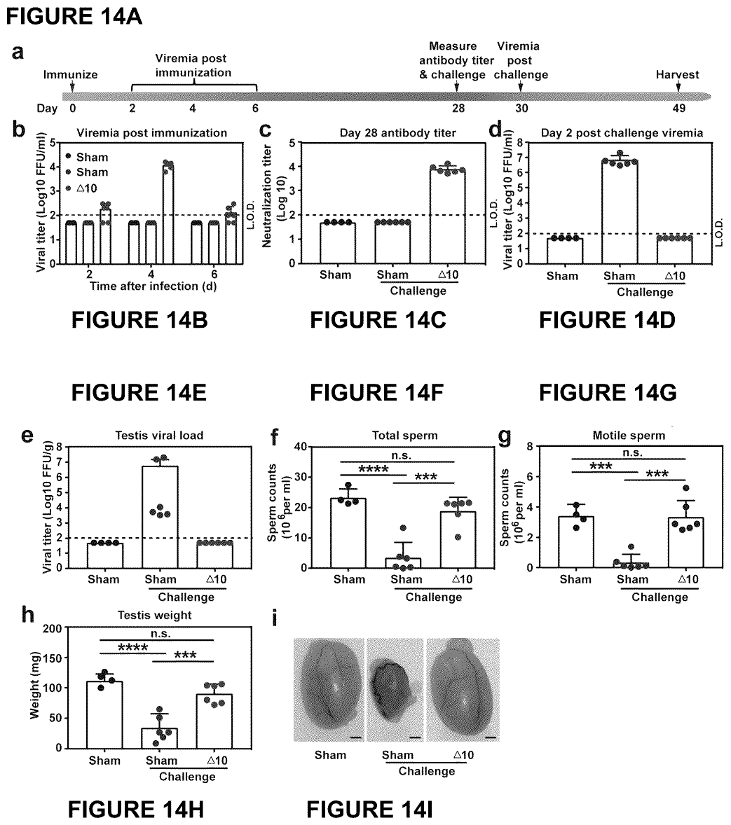

We examined the ability of ZIKV-3′UTR-Δ10-LAV to prevent testis infection and injury in Ifnar1^{-/-} A129 mice (FIG. 14A). Since male mice reach sexual maturity at 8-week-old, we tested the vaccine efficacy in two age groups of mice: 3-week-old young males and 15-week-old adult males. Three-week-old young A129 male mice were vaccinated with a single-dose of 10⁴ FFU of ZIKV-3′UTR-Δ10-LAV or PBS sham. The ZIKV-3′UTR-Δ10-LAV generated a peak viremia of 1.2±0.34×10⁴ FFU (n=6) at day 4 post-vaccination (FIG. 14B). At day 28 post-vaccination, ZIKV-3′UTR-Δ10-LAV had induced robust neutralizing antibody titers of 7,700±2,600 (n=6) (FIG. 14C). At the same day, the animals were challenged intraperitoneally with 10⁶ FFU of an epidemic ZIKV strain from Puerto Rico (PRVABC59). No viremia was detected from the ZIKV-3′UTR-Δ10-LAV-vaccinated mice after challenge, whereas the sham-vaccinated animals sustained a mean peak viremia of 7.1±5.9×10⁶ FFU/ml (n=6) at day 2 post-challenge (FIG. 14D). At day 21 post-challenge, viral burden in the testis of PBS-immunized mice reached 5.8±8.4×10⁶ FFU/g (n=6), whereas no infectious virus was detected in the testes of ZIKV-3′UTR-Δ10-LAV-immunized mice (FIG. 14E). Both total and motile sperm counts from the ZIKV-3′UTR-Δ10-LAV-immunized mice were equivalent to those from age-matched unvaccinated, unchallenged healthy male mice. In contrast, the PBS-immunized, ZIKV-challenged mice showed 85% and 90% reduction for total and motile sperm counts, respectively, at day 21 post-infection (FIGS. 14F&G). Consistent with these data, the testis weight and size from the PBS-immunized, ZIKV-challenged mice were reduced, whereas no such reduction was observed in the ZIKV-3′UTR-Δ10-LAV-immunized, ZIKV-challenged animals (FIGS. 14H&I).

In 15-week-old adult A129 male mice, vaccination with ZIKV-3′UTR-Δ10-LAV also protected against testis infection, injury, and oligospermia (FIG. 17A-G). However, challenge of the PBS-vaccinated adult males with ZIKV PRVABC59 did not reduce the testis weight and size (FIGS. 17H&I) as much as that observed in the corresponding sham-vaccinated, young males (FIGS. 17H&I), suggesting an age-dependent testis pathology. Collectively, the results indicate that a single-dose immunization of ZIKV-3′UTR-Δ10-LAV protects the testis from infection and injury in male mice.

### Example 11: Efficacy in Non-human Primates

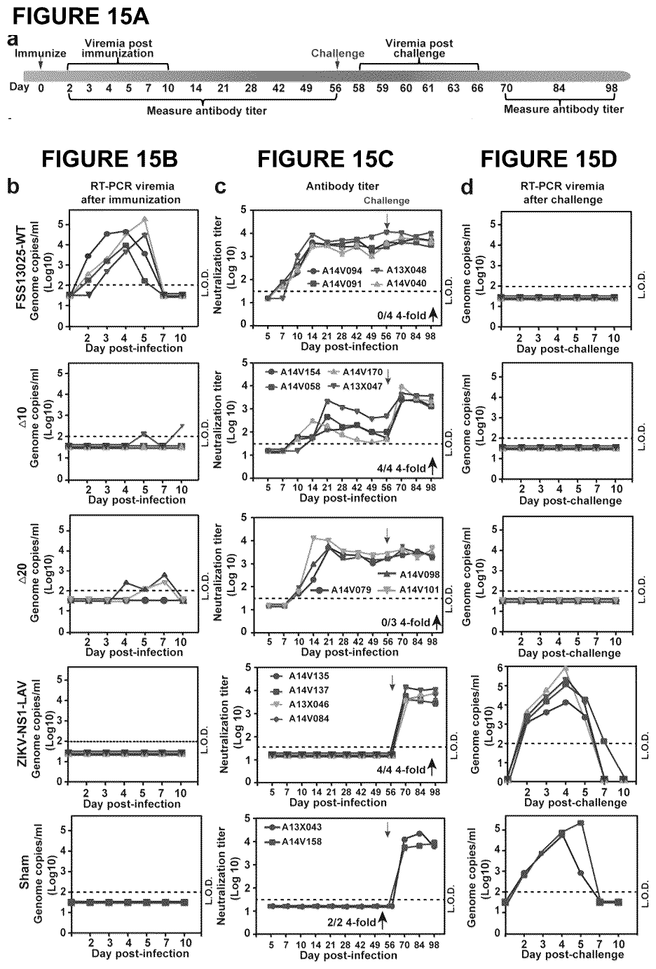

To determine whether the efficacy in mice extends to non-human primates (NHPs), we evaluated the viremia, immunogenicity, and potency of ZIKV-3′UTR-Δ10-LAV in rhesus macaques (RM; FIG. 15A). First, we assessed the level of attenuation of ZIKV-3′UTR-Δ10-LAV in RM by comparing viremia to the parental WT virus. After subcutaneous inoculation with 10³ FFU, WT ZIKV (2010 Cambodian strain FSS 13025) produced high levels of viremia in each of the four inoculated RM, with a mean peak viremia of 9.6 and 28.8×10⁴ genome copies/ml at days 4 and 5 post-infection, respectively (FIG. 15B, top panel). In contrast, only one of the four ZIKV-3′UTR-Δ10-LAV-inoculated RM exhibited viremia, and this level was just above the limit of detection of our qRT-PCR assay (FIG. 15B, second panel); thus, ZIKV-3′UTR-Δ10-LAV is highly attenuated in RM.

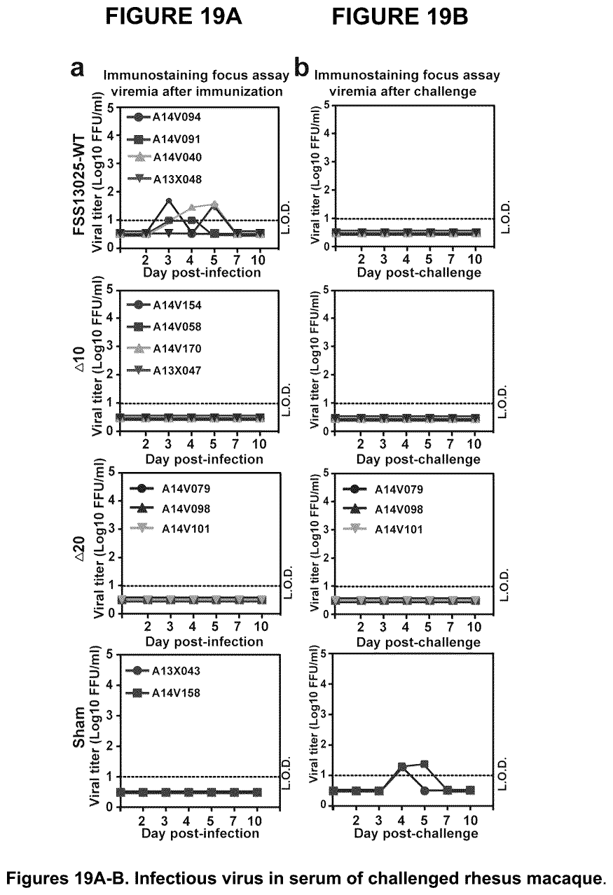

We next evaluated the immunogenicity of ZIKV-3′UTR-Δ10-LAV by measuring neutralizing antibodies from serum at days 5-98 post-immunization. Neutralizing antibodies elicited by WT ZIKV were detectable at day 7-10, peaked at 1/1,000 to 1/10,000 at day 14, and plateaued thereafter (FIG. 15C, top panel). Compared with the WT ZIKV infection, ZIKV-3′UTR-Δ10-LAV-inoculated animals showed slightly delayed production and lower levels of neutralizing antibody, with titers of ˜1/100 at days 21-56 (FIG. 15C, second panel). At day 56 post-immunization, all RM were challenged with 10³ FFU of the epidemic ZIKV strain PRVABC59. Notably, no viremia was detected upon challenge in any of the RM that were pre-infected with WT ZIKV or vaccinated with the ZIKV-3′UTR-Δ10-LAV (FIG. 15D, top two panels). As controls, two PBS-inoculated RM were challenged in parallel and high levels of viremia were measured (FIG. 15D bottom panel). In addition to analysis of viral RNA by qRT-PCR, we also performed focus forming assays to measure infectious virus; infectious ZIKV was detected only in naïve RM challenged with WT ZIKV (FIG. 19).

To examine whether the ZIKV challenge resulted in boosted immune responses, we measured neutralizing activity post-challenge. No increase in neutralizing activity was observed in the WT ZIKV-vaccinated RM after challenge (FIG. 15C, top panel), indicating that the initial infection likely conferred sterilizing immunity. In contrast, the neutralizing antibody titers rose after challenge from approximately 1/100 to 1/1,000-1/10,000 in the ZIKV-3′UTR-Δ10-LAV-immunized animals (FIG. 15C, second panel), demonstrating an anamnestic response and suggesting a low level of infection after challenge that was not detectable by qRT-PCR of serum. As expected, the PBS-inoculated control RM increased their neutralizing titers to ˜1/10,000 after challenge (FIG. 15C, bottom panel).

Since ZIKV-3′UTR-Δ10-LAV did not elicit sterilizing immunity in RM, we evaluated whether a second live-attenuated vaccine candidate ZIKV-3′UTR-Δ20-LAV (Reference 36), which contains a 20-nucleotide deletion in the 3′UTR, could induce a stronger immune response. ZIKV-3′UTR-Δ20-LAV was shown previously, and paradoxically, to be less attenuated than ZIKV-3′UTR-Δ10-LAV in A129 mice, most likely because it is less sensitive to type-I IFN inhibition compared to ZIKV-3′UTR-Δ10-LAV (Reference 36). After subcutaneous inoculation of 10³ FFU of ZIKV-3′UTR-Δ20-LAV, two of the three RM had low, but detectable viremia (FIG. 15B, third panel). The immunized animals rapidly produced neutralizing antibodies by day 10, with inhibitory titers plateauing at 1/1,000 to 1/10,000 by days 14-21 (FIG. 15C, third panel). After challenge with 10³ FFU of ZIKV PRVABC59 at day 56, viremia was not detected by qRT-PCR (FIG. 15D, third panel) and no rise in neutralizing antibody titers was observed (FIG. 15C, third panel) in the ZIKV-3′UTR-Δ20-LAV-immunized animals. Although low number of animals were used for each vaccine candidates, the results suggest that a single-dose vaccination of ZIKV-3′UTR-Δ20-LAV-induces sterilizing immunity in NHPs (i.e., no detectable viremia and no increase of neutralizing antibody titer after challenge).

We also evaluated another live-attenuated ZIKV vaccine candidate encoding an NS1 without glycosylation (ZIKV-NS1-LAV) in RM. ZIKV-NS1-LAV was recently shown to prevent in utero transmission in a mouse pregnancy model (Reference 43). After subcutaneous immunization of four RMs with 10³ FFU of ZIKV-NS1-LAV, none of the animals showed any detectable viral RNA (FIG. 15B, fourth panel). Back titering of the ZIKV-NS1-LAV inoculum using focus-forming assay confirmed the infectivity of viral stock with the expected infectious titer. Unexpectedly the immunization did not elicit any neutralizing activity (FIG. 15C, fourth panel). After challenge with 10³ FFU of ZIKV PRVABC59 at day 56, all four animals displayed robust viremia (FIG. 15D, fourth panel) and generated neutralizing antibody titers (FIG. 15C, fourth panel). These results indicate that ZIKV-NS1-LAV is incapable of replicating and triggering antibody responses in RM.

### Example 12: Testis Protection and Safety Analysis of ZIKV-3′UTR-Δ20-LAV

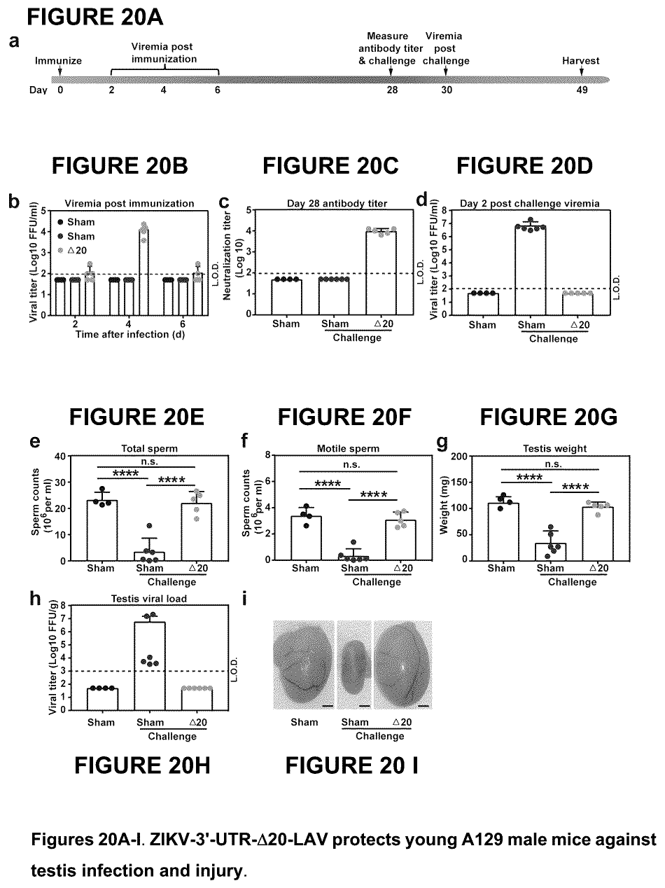

Because of the highly desirable sterilizing immunity induced by ZIKV-3′UTR-Δ20-LAV in RM, we further tested its efficacy and safety. Similar to ZIKV-3′UTR-Δ10-LAV, immunization of male A129 mice with 10³ FFU of ZIKV-3′UTR-Δ20-LAV completely prevented viral infection and testis injury after challenge with ZIKV PRVABC59, as determined by a lack of detectable viremia post-challenge, the absence of oligospermia, and no decrease in testis weight and size (FIG. 20). Next, five sets of experiments were performed to characterize the safety of ZIKV-3′UTR-Δ20-LAV.

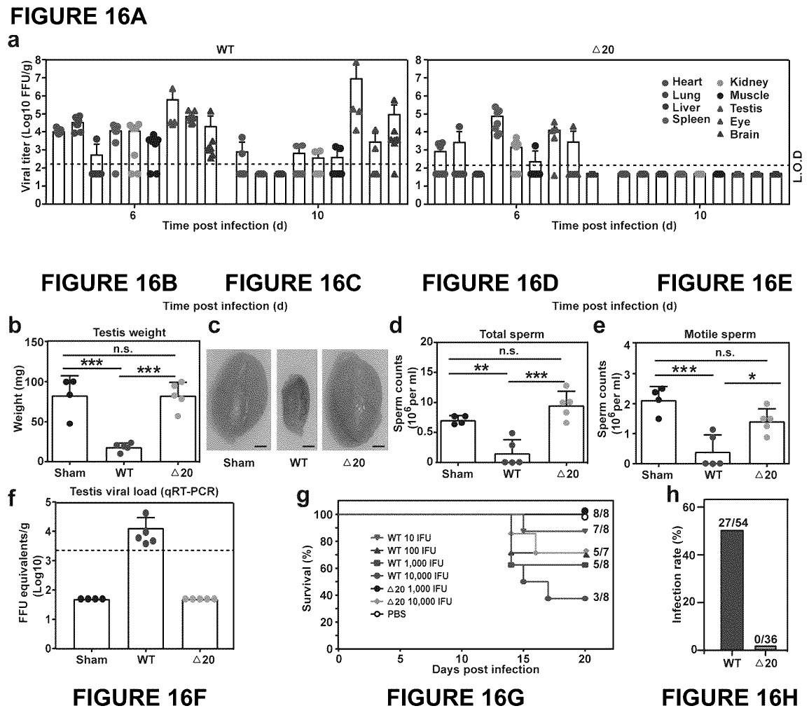

First, we measured the organ viral loads after subcutaneous inoculation of A129 mice with 10³ FFU of ZIKV-3′UTR-Δ20-LAV or parental WT ZIKV (FIG. 16A). At day 6 post-infection, WT ZIKV-infected mice exhibited high viral loads in all tested organs, whereas no virus was detected (≤10² FFU/ml) in liver or brain from the ZIKV-3′UTR-Δ20-LAV-infected mice, with other organs (except spleen) exhibiting lower levels of the vaccine virus than those of the WT-infected animals. At day 10 post-infection, WT ZIKV-infected mice retained viral loads in the heart, spleen, kidney, testis, eye, and brain, whereas no organs from the ZIKV-3′UTR-Δ20-LAV-infected mice had any detectable virus.

Second, we examined the potential adverse effect of ZIKV-3′UTR-Δ20-LAV on the testis in 3-week-old A129 mice. As expected, at day 21 post-infection, WT ZIKV infection reduced testis weight and size (FIG. 16A-C), lowered total and motile sperm counts (FIG. 16D-E), and resulted in viral RNA in the shrunken testis (FIG. 16F). In contrast, ZIKV-3′UTR-Δ20-LAV did not affect sperm counts or testis weight and size (FIG. 16B-E), with no detectable viral RNA in the testes (FIG. 16F).

Third, we evaluated the neurovirulence of ZIKV-3′UTR-Δ20-LAV through intracranial inoculation of 1-day-old CD-1 mice (FIG. 16G). As reported previously (Reference 36), neonates succumbed to WT ZIKV infection; even a dose of only 10 FFU resulted in 13% mortality (FIG. 16G). In contrast, no mortality was observed in mice that were inoculated with 10³ FFU of ZIKV-3′UTR-Δ20-LAV; however, infection with 10⁴ FFU of ZIKV-3′UTR-Δ20-LAV resulted in a mortality rate of 29%.

Fourth, we tested if the vaccine candidate could infect Aedes aegypti mosquitoes, the main vector of ZIKV (References 19, 20). After feeding on artificial blood-meals containing 10⁶ FFU/ml of ZIKV-3′UTR-Δ20-LAV or WT ZIKV, 50% of the engorged mosquitoes were infected by WT ZIKV, whereas no mosquitoes were infected by ZIKV-3′UTR-Δ20-LAV (FIG. 16H).

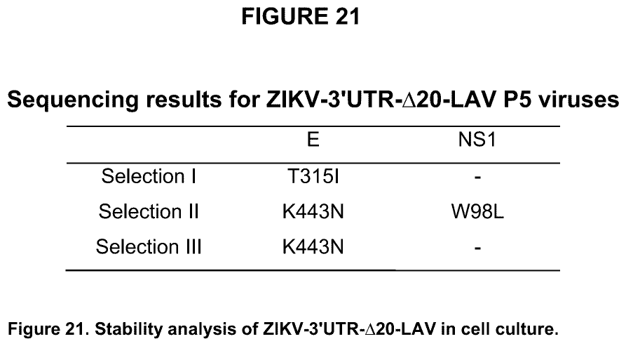

Finally, we tested the stability of ZIKV-3′UTR-Δ20-LAV in cell culture. After continuous culture of ZIKV-3′UTR-Δ20-LAV on Vero cells (an approved cell line for vaccine production (Reference 44) for five rounds, all recovered P5 viruses (derived from three independent experiments) retained the 20-nucleotide deletion. However, the P5 viruses accumulated additional mutations in the E- and NS1-encoding genes (FIG. 21), which may represent Vero-cell-adaptive mutation(s) or compensatory mutation(s) to 3′UTR deletion. Further passaging of the viruses to P10 did not change the 20-nucleotide deletion, indicating that the deletion is stable in cell culture. Moreover, we passaged ZIKV-3′UTR-Δ20-LAV in A129 mice for three rounds (3 days per round); all recovered viruses retained the 20-nucleotide deletion, further suggesting the stability of the mutant virus. Taken together, these results demonstrate an excellent safety profile of ZIKV-3′UTR-Δ20-LAV, including limited, transient viral loads in mouse organs, no adverse effect on testicular function, decreased neurovirulence, incompetency to infect mosquitoes, and good stability.

### Conclusions

Herein we demonstrate that live-attenuated ZIKV vaccine candidates containing deletions in the 3′ untranslated region of the ZIKV genome (ZIKV-3′UTR-LAV), i.e., 10 and 20 nucleotide deletions, prevent viral transmission during pregnancy and testis damage in mice, as well as infection of non-human primates. After a single-dose vaccination, pregnant mice challenged with ZIKV at embryonic day 6 (E6) and evaluated at E13 show markedly diminished levels of viral RNA in maternal, placental, and fetal tissues. Vaccinated male mice challenged with ZIKV are protected against testis infection, injury, and oligospermia. A single immunization of rhesus macaques elicited a rapid and robust antibody response, conferring complete protection upon challenge. Furthermore, the ZIKV-3′UTR-LAV vaccine candidates have a desirable safety profile. These results suggest that further development of ZIKV-3′UTR-LAV is warranted for humans.

Particularly our results showed that a single immunization of ZIKV-3′UTR-Δ10-LAV prevented maternal-to-fetal transmission early during pregnancy in C57BL/6 mice. Although no infectious challenge virus was detected, very low levels of viral RNA were recovered from ˜30% of placenta and fetal heads from the vaccinated dams after challenge; these breakthrough viral RNAs might derive from stable antibody-virus complexes, which can last for several days in vivo (Reference 47).

Based on these results the clinical implications of such breakthrough non-infectious viral RNA will be determined in other species and in particular will be evaluated in non-human primates (NHPs) and if successful in humans. In male A129 mice, a single-dose immunization of either ZIKV-3′UTR-Δ10-LAV or ZIKV-3′UTR-Δ20-LAV prevented testis infection and injury after challenge, indicating an additional benefit of vaccination to protect the male reproductive system. Notably, unprotected young A129 mice (3- or 7-week-old) infected with ZIKV developed smaller testes, whereas adult mice (19-week-old when infected) did not, suggesting that ZIKV infection might cause more severe reproductive damage in younger males. As noted above the clinical relevance of this observation will be confirmed in NHPs and humans.

In NHPs, a single-dose vaccination with ZIKV-3′UTR-Δ10-LAV or ZIKV-3′UTR-Δ20-LAV induced sufficient immune responses to prevent viremia, with the ZIKV-3′UTR-Δ10-LAV eliciting greater immunogenicity, as reflected by its ability to induce sterilizing immunity against challenge. One limitation of the current non-human primate results is the low number of animals used for each vaccine candidates (n=3−4). ZIKV vaccine-induced sterilizing immunity might be critical for protection of congenital abnormalities in humans.

Live-attenuated vaccines generally have the advantage of single dose, rapid induction of durable immunity. Since ZIKV is endemic primarily in low income countries, a vaccine with single-dose efficacy is of practical importance, particularly when controlling an explosive outbreak or immunizing population in remote areas where multiple doses and periodic boosting will be challenging (Reference 45). Thus, live-attenuated vaccines may be useful for immunizing populations living in and traveling to ZIKV-endemic areas. Besides our ZIKV-3′UTR-LAV, a single-dose immunization with nucleoside-modified mRNA expressing ZIKV prM-E (50 μg) (Reference 46) or a recombinant rhesus adenovirus serotype 52 vector expressing ZIKV prM-E (10¹¹ viral particles) (Reference 8) was also shown to rapidly elicit antibody response and prevent viremia in NHPs; whether these two vaccines achieved sterilizing immunity was not determined. All other vaccine platforms, including inactivated vaccine and prM-E DNA vaccine, need two shots to elicit robust antibody response for viremia protection in NHPs (References 7, 8).

It is conceivable that in immunocompromised individuals and pregnant women, vaccination with live-attenuated virus may be contraindicated to avoid potential adverse risks. However, these individuals could be protected using inactivated, subunit, or gene-based replication-defective vaccines. Therefore, it is desirable that multiple vaccine platforms be developed in parallel with those described herein in order to provide complementary options for preventing and controlling ZIKV infection and disease.

### Example 13: DNA Plasmid-launched Wild-type Zika Virus and Vaccines

In order to facilitate the development of vaccines for preventing and controlling ZIKV infection and disease the inventors have developed a DNA plasmid that can be directly transfected into cells to generate Zika virus (ZIKV). The new DNA-launched ZIKV full-length (FL) clone is assembled in the backbone of the pCC1 vector. Besides the pCC1 vector sequence, it contains an eukaryotic promoter CMV or SV40, the full genome of ZIKV strain FSS13025, HDVr sequence, and poly-A tail. The clones are named as CMV ZIKV FL and SV40 ZIKV FL depending on the types of promoters used to transcribe the viral RNA. DNA-launched ZIKV full-length clones used to construct live attenuated ZIKV vaccine candidates are listed in Appendix A.

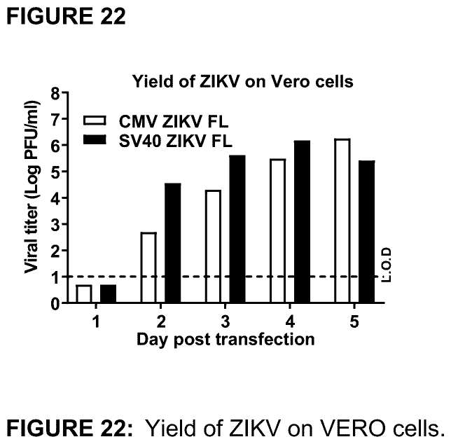

As shown in FIG. 22, both pCC1-CMV ZIKV FL and pCC1-SV40 ZIKV FL can efficiently launch wild-type ZIKV by transfection of DNA plasmid into Vero cells. In the experiments 5 micrograms of indicated DNA plasmid was transfected into Vero cells through electroporation. Culture fluids were collected from day 1 to 5. Infectious viral titers were measured by plaque assay on Vero cells.

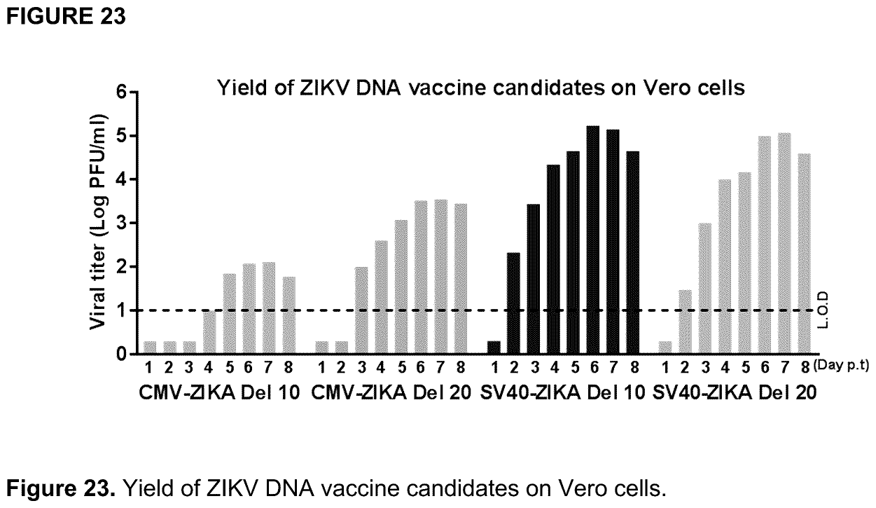

Using the DNA-launched ZIKV FL clones (SEQ ID NO:6 and 7), we made DNA-launched live attenuated ZIKV vaccine candidates. Specifically, we engineered the 3′UTR 10-del or 20-del mutations (References 19, 48) into the DNA-launched ZIKV FL clones, resulting in ZIKV Del 10 and ZIKV Del 20, respectively. Depending on the promoter types, these DNA vaccine candidates are named as CMV ZIKV Del 10, CMV ZIKV Del 20, SV40 ZIKV Del 10, and SV40 ZIKV Del 20. Detailed description of the sequences of these particular DNA vaccine candidates is summarized in Appendix B. As shown in FIG. 23, the four DNA-launched live-attenuated ZIKV vaccine plasmids can also produce robust levels of vaccines after transfecting the DNA into Vero cells.

### Appendix A: ZIKV Full-length Clones Used to Construct Live Attenuated ZIKV Vaccine Candidates

1. CMV ZIKV Full Length (FL) sequence: SEQ ID NO: 6 in Sequence Listing
2. SV40 ZIKV Full Length (FL) sequence: SEQ ID NO:7 in Sequence Listing

### Appendix B: Detailed Description of Sequences of Exemplary Live Attenuated ZIKV Vaccine Candidates

CMV ZIKV Del 10: Consists of a deletion variant of SEQ ID NO: 6 wherein the sequence “CCAGAAGAGG” (SEQ ID NO: 8 in Sequence Listing) is deleted.
CMV ZIKV Del 20: Consists of a deletion variant of SEQ ID NO: 6 wherein the sequence “CTGTGGATCTCCAGAAGAGG” (SEQ ID NO: 9 in Sequence Listing) is deleted.
SV40 ZIKV Del 10: Consists of a deletion variant of SEQ ID NO: 7 wherein the sequence “CCAGAAGAGG” (SEQ ID NO: 8 in Sequence Listing) is deleted.
SV40 ZIKV Del 10: Consists of a deletion variant of SEQ ID NO: 7 wherein the sequence “CTGTGGATCTCCAGAAGAGG” (SEQ ID NO: 9 in Sequence Listing) is deleted.

One skilled in the art will readily appreciate that the present invention is adapted to carry out the objects and obtain the ends and advantages mentioned, as well as those inherent therein. The prior examples along with the methods, procedures, treatments, molecules, and specific compounds described herein are presently representative of preferred embodiments, are examples, and are not intended as limitations on the scope of the invention. Changes therein and other uses will occur to those skilled in the art which are encompassed within the spirit of the invention as defined by the scope of the claims.

SEQUENCES
3′UTR-WT
SEQ ID NO: 1
GCACCAATCTTAGTGTTGTCAGGCCTGCTAGTCAGCCACAGCTTGGGGAAAGCTG
TGCAGCCTGTGACCCCCCCAGGAGAAGCTGGGAAACCAAGCCCATAGTCAGGCC
GAGAACGCCATGGCACGGAAGAAGCCATGCTGCCTGTGAGCCCCTCAGAGGACA
CTGAGTCAAAAAACCCCACGCGCTTGGAGGCGCAGGATGGGAAAAGAAGGTGG
CGACCTTCCCCACCCTTTAATCTGGGGCCTGAACTGGAGATCAGCTGTGGATCTC
CAGAAGAGGGACTAGTGGTTAGAGGAGACCCCCCGGAAAACGCAAAACAGCAT
ATTGACGCTGGGAAAGACCAGAGACTCCATGAGTTTCCACCACGCTGGCCGCCA
GGCACAGATCGCCGAATAGCGGCGGCCGGTGTGGGGAAATCCATGGTTTCT
3′UTR-10-del
SEQ ID NO: 2
GCACCAATCTTAGTGTTGTCAGGCCTGCTAGTCAGCCACAGCTTGGGGAAAGCTG
TGCAGCCTGTGACCCCCCCAGGAGAAGCTGGGAAACCAAGCCCATAGTCAGGCC
GAGAACGCCATGGCACGGAAGAAGCCATGCTGCCTGTGAGCCCCTCAGAGGACA
CTGAGTCAAAAAACCCCACGCGCTTGGAGGCGCAGGATGGGAAAAGAAGGTGG
CGACCTTCCCCACCCTTTAATCTGGGGCCTGAACTGGAGATCAGCTGTGGATCTG
ACTAGTGGTTAGAGGAGACCCCCCGGAAAACGCAAAACAGCATATTGACGCTGG
GAAAGACCAGAGACTCCATGAGTTTCCACCACGCTGGCCGCCAGGCACAGATCG
CCGAATAGCGGCGGCCGGTGTGGGGAAATCCATGGTTTCT
3′UTR-20-del
SEQ ID NO: 3
GCACCAATCTTAGTGTTGTCAGGCCTGCTAGTCAGCCACAGCTTGGGGAAAGCTG
TGCAGCCTGTGACCCCCCCAGGAGAAGCTGGGAAACCAAGCCCATAGTCAGGCC
GAGAACGCCATGGCACGGAAGAAGCCATGCTGCCTGTGAGCCCCTCAGAGGACA
CTGAGTCAAAAAACCCCACGCGCTTGGAGGCGCAGGATGGGAAAAGAAGGTGG
CGACCTTCCCCACCCTTTAATCTGGGGCCTGAACTGGAGATCAGGACTAGTGGTT
AGAGGAGACCCCCCGGAAAACGCAAAACAGCATATTGACGCTGGGAAAGACCA
GAGACTCCATGAGTTTCCACCACGCTGGCCGCCAGGCACAGATCGCCGAATAGC
GGCGGCCGGTGTGGGGAAATCCATGGTTTCT
3′UTR-30-del-a
SEQ ID NO: 4
GCACCAATCTTAGTGTTGTCAGGCCTGCTAGTCAGCCACAGCTTGGGGAAAGCTG
TGCAGCCTGTGACCCCCCCAGGAGAAGCTGGGAAACCAAGCCCATAGTCAGGCC
GAGAACGCCATGGCACGGAAGAAGCCATGCTGCCTGTGAGCCCCTCAGAGGACA
CTGAGTCAAAAAACCCCACGCGCTTGGAGGCGCAGGATGGGAAAAGAAGGTGG
CGACCTTCCCCACCCTTTAATCTGGGGCCTGAACGACTAGTGGTTAGAGGAGACC
CCCCGGAAAACGCAAAACAGCATATTGACGCTGGGAAAGACCAGAGACTCCATG
AGTTTCCACCACGCTGGCCGCCAGGCACAGATCGCCGAATAGCGGCGGCCGGTG
TGGGGAAATCCATGGTTTCT
3′UTR-30-del-b
SEQ ID NO: 5
GCACCAATCTTAGTGTTGTCAGGCCTGCTAGTCAGCCACAGCTTGGGGAAAGCTG
TGCAGCCTGTGACCCCCCCAGGAGAAGCTGGGAAACCAAGCCCATAGTCAGGCC
GAGAACGCCATGGCACGGAAGAAGCCATGCTGCCTGTGAGCCCCTCAGAGGACA
CTGAGTCAAAAAACCCCACGCGCTTGGAGGCGCAGGATGGGAAAAGAAGGTGG
CGACCTTCCCCACCCTTTAATCTGGGGCCTGAACTGGAGATCAGCTGTGGAGACC
CCCCGGAAAACGCAAAACAGCATATTGACGCTGGGAAAGACCAGAGACTCCATG
AGTTTCCACCACGCTGGCCGCCAGGCACAGATCGCCGAATAGCGGCGGCCGGTG
TGGGGAAATCCATGGTTTCT
CMV ZIKV FL sequence:
SEQ ID NO: 6
AGTTGTTGATCTGTGTGAATCAGACTGCGACAGTTCGAGTTTGAAGCGAAAGCTA
GCAACAGTATCAACAGGTTTTATTTTGGATTTGGAAACGAGAGTTTCTGGTCATG
AAAAACCCAAAGAAGAAATCCGGAGGATTCCGGATTGTCAATATGCTAAAACGC
GGAGTAGCCCGTGTGAGCCCCTTTGGGGGCTTGAAGAGGCTGCCAGCCGGACTT
CTGCTGGGTCATGGGCCCATCAGGATGGTCTTGGCGATTCTAGCCTTTTTGAGAT
TCACGGCAATCAAGCCATCACTGGGTCTCATCAATAGATGGGGTTCAGTGGGGA
AAAAAGAGGCTATGGAAATAATAAAGAAGTTTAAGAAAGATCTGGCTGCCATGC
TGAGAATAATCAATGCTAGGAAGGAGAAGAAGAGACGAGGCACAGATACTAGT
GTCGGAATTGTTGGCCTCCTGCTGACCACAGCCATGGCAGTGGAGGTCACTAGAC
GTGGGAATGCATACTATATGTACTTGGACAGAAGCGATGCTGGGGAGGCCATAT
CTTTTCCAACCACAATGGGGATGAATAAGTGTTATATACAGATCATGGATCTTGG
ACACATGTGTGATGCCACCATGAGCTATGAATGCCCTATGCTGGATGAGGGGGT
AGAACCAGATGACGTCGATTGTTGGTGCAACACGACGTCAACTTGGGTTGTGTAC
GGAACCTGCCACCACAAAAAAGGTGAAGCACGGAGATCTAGAAGAGCTGTGAC
GCTCCCCTCCCATTCCACTAGGAAGCTGCAAACGCGGtCGCAGACCTGGTTGGAA
TCAAGAGAATACACAAAGCACCTGATTAGAGTCGAAAATTGGATATTCAGGAAC
CCTGGCTTCGCGTTAGCAGCAGCTGCCATCGCTTGGCTTTTGGGAAGCTCAACGA
GCCAAAAAGTCATATACTTGGTCATGATACTGCTGATTGCCCCGGCATACAGCAT
CAGGTGCATAGGAGTCAGCAATAGGGACTTTGTGGAAGGTATGTCAGGTGGGAC
TTGGGTTGATGTTGTCTTGGAACATGGAGGTTGTGTTACCGTAATGGCACAGGAC
AAACCGACTGTCGACATAGAGCTGGTTACAACAACAGTCAGCAACATGGCGGAG
GTAAGATCCTACTGCTATGAGGCATCAATATCGGACATGGCTTCGGACAGCCGCT
GCCCAACACAAGGTGAAGCCTACCTTGACAAGCAATCAGACACTCAATATGTCT
GCAAAAGAACGTTAGTGGACAGAGGCTGGGGAAATGGATGTGGACTTTTTGGCA
AAGGGAGCCTGGTGACATGCGCTAAGTTTGCTTGCTCTAAGAAAATGACCGGGA
AGAGCATCCAGCCAGAGAATCTGGAGTACCGGATAATGCTGTCAGTTCATGGCT
CCCAGCACAGTGGGATGATCGTTAATGATACAGGACATGAAACTGATGAGAATA
GAGCGAAGGTTGAGATAACGCCCAATTCACCAAGAGCCGAAGCCACCCTGGGGG
GTTTTGGAAGCCTAGGACTTGATTGTGAACCGAGGACAGGCCTTGACTTTTCAGA
TTTGTATTACTTGACTATGAATAACAAGCACTGGTTGGTTCACAAGGAGTGGTTC
CACGACATTCCATTACCTTGGCAcGCTGGGGCAGACACCGGAACTCCACACTGGA
ACAACAAAGAAGCACTGGTAGAGTTCAAGGACGCACATGCCAAAAGGCAGACT
GTCGTGGTTCTAGGGAGTCAAGAAGGAGCAGTTCACACGGCCCTTGCTGGAGCT
CTGGAGGCTGAGATGGATGGTGCAAAGGGAAGGCTGTCCTCTGGCCACTTGAAA
TGTCGCCTGAAAATGGAcAAACTTAGATTGAAGGGCGTGTCATACTCCTTGTGTA
CCGCAGCGTTCACATTCACTAAGATCCCGGCTGAAACACTGCACGGGACAGTCA
CAGTGGAGGTACAGTACGCAGGGACAGATGGACCTTGCAAGGTTCCAGCTCAGA
TGGCGGTGGACATGCAAACTCTGACCCCAGTTGGGAGGTTGATAACCGCTAACC
CTGTAATCACTGAAAGCACTGAGAACTCCAAGATGATGCTGGAACTGGATCCAC
CATTTGGGGACTCTTACATTGTCATAGGAGTCGGGGAAAAGAAGATCACCCACC
ACTGGCACAGGAGTGGCAGCACCATTGGAAAAGCATTTGAAGCCACTGTGAGAG
GTGCCAAGAGAATGGCAGTCTTGGGAGACACAGCCTGGGACTTTGGATCAGTTG
GGGGTGCTCTCAACTCACTGGGCAAGGGCATCCATCAAATTTTTGGAGCAGCTTT
CAAATCATTGTTTGGAGGAATGTCCTGGTTCTCACAAATTCTCATTGGAACGTTG
CTGGTGTGGTTGGGTCTGAATACAAAGAATGGATCTATTTCCCTTATGTGCTTGG
CCTTAGGGGGAGTGTTGATCTTCTTATCCACAGCCGTCTCTGCTGATGTGGGGTG
CTCGGTGGACTTCTCAAAGAAGGAAACGAGATGCGGTACAGGGGTGTTCGTCTA
TAACGACGTTGAAGCTTGGAGGGACAGGTACAAGTACCATCCTGACTCCCCTCGT
AGATTGGCAGCAGCAGTCAAGCAAGCCTGGGAAGATGGGATCTGTGGGATCTCC
TCTGTTTCAAGAATGGAAAACATCATGTGGAGATCAGTAGAAGGGGAGCTCAAC
GCAATCCTGGAAGAGAATGGAGTTCAACTGACGGTCGTTGTGGGATCTGTAAAA
AACCCCATGTGGAGAGGTCCACAGAGATTGCCCGTGCCTGTGAACGAGCTGCCC
CATGGCTGGAAGGCTTGGGGGAAATCGTACTTCGTCAGGGCAGCAAAGACAAAT
AACAGCTTTGTCGTGGATGGTGACACACTGAAGGAATGCCCACTCAAACATAGA
GCATGGAACAGCTTTCTTGTGGAGGATCATGGGTTCGGGGTATTTCACACTAGTG
TCTGGCTCAAGGTTAGAGAAGATTATTCACTCGAGTGTGATCCAGCCGTCATTGG
AACAGCCGCTAAGGGAAAGGAGGCTGTGCACAGTGATCTAGGCTACTGGATTGA
GAGTGAGAAGAACGACACATGGAGGCTGAAGAGGGCCCACCTGATCGAGATGA
AAACATGTGAATGGCCAAAGTCCCACACATTGTGGACAGATGGAATAGAAGAAA
GTGATCTGATCATACCCAAGTCTTTAGCTGGGCCACTCAGCCATCACAACACCAG
AGAGGGCTACAGGACCCAAATGAAAGGGCCATGGCATAGTGAAGAGCTTGAAAT
TCGGTTTGAGGAATGCCCAGGCACTAAGGTCCACGTGGAGGAAACATGTGGAAC
AAGAGGACCATCTCTGAGATCAACCACTGCAAGCGGAAGGGTGATCGAGGAATG
GTGCTGCAGGGAGTGCACAATGCCCCCACTGTCGTTCCGGGCTAAAGATGGTTGT
TGGTATGGAATGGAGATAAGGCCCAGGAAAGAACCAGAAAGTAACTTAGTAAG
GTCAATGGTGACTGCAGGATCAACTGATCACATGGATCACTTCTCCCTTGGAGTG
CTTGTGATTCTGCTCATGGTACAGGAAGGGCTAAAGAAGAGAATGACCACAAAG
ATCATCATAAGCACATCAATGGCAGTGCTGGTAGCTATGATCCTGGGAGGATTTT
CAATGAGTGACCTGGCTAAGCTTGCAATTTTGATGGGTGCCACCTTCGCGGAAAT
GAACACTGGAGGAGATGTTGCTCATCTGGCGCTGATAGCGGCATTCAAAGTCAG
ACCTGCGTTGCTGGTATCTTTCATTTTCAGAGCTAATTGGACACCCCGTGAGAGC
ATGCTGCTGGCCTTGGCCTCGTGTCTTCTGCAAACTGCGATCTCCGCCTTGGAAG
GCGACCTGATGGTTCCCATCAATGGTTTTGCTTTGGCCTGGTTGGCAATACGAGC
GATGGTTGTTCCACGCACTGACAACATCACCTTGGCAATCCTGGCTGCTCTGACA
CCACTGGCCCGGGGCACACTGCTTGTGGCGTGGAGAGCAGGCCTTGCTACTTGCG
GGGGGTTCATGCTCCTTTCTCTGAAGGGGAAAGGCAGTGTGAAGAAGAACTTAC
CATTTGTCATGGCCCTGGGACTAACCGCTGTGAGGCTGGTCGACCCCATCAACGT
GGTGGGACTGCTGTTGCTCACAAGGAGTGGGAAGCGGAGCTGGCCCCCTAGTGA
AGTACTCACAGCTGTTGGCCTGATATGCGCATTGGCTGGAGGGTTCGCCAAGGCG
GATATAGAGATGGCTGGGCCCATGGCCGCGGTCGGTCTGCTAATTGTCAGTTACG
TGGTCTCAGGAAAGAGTGTGGACATGTACATTGAAAGAGCAGGTGACATCACAT
GGGAAAAAGATGCGGAAGTCACTGGAAACAGTCCCCGGCTCGATGTGGCACTAG
ATGAGAGTGGTGATTTCTCCCTAGTGGAGGATGATGGTCCCCCCATGAGAGAGAT
CATACTCAAAGTGGTCCTGATGGCCATCTGTGGCATGAACCCAATAGCCATACCC
TTTGCAGCTGGAGCGTGGTACGTGTATGTGAAGACTGGAAAAAGGAGTGGTGCT
CTATGGGATGTGCCTGCTCCCAAGGAAGTAAAAAAGGGGGAGACCACAGATGGA
GTGTACAGAGTAATGACTCGTAGACTGCTAGGTTCAACACAAGTTGGAGTGGGA
GTCATGCAAGAGGGGGTCTTCCACACTATGTGGCACGTCACAAAAGGATCCGCG
CTGAGAAGCGGTGAAGGGAGACTTGATCCATACTGGGGAGATGTCAAGCAGGAT
CTGGTGTCATACTGTGGTCCATGGAAGCTAGATGCCGCCTGGGACGGGCACAGC
GAGGTGCAGCTCTTGGCCGTGCCCCCCGGAGAGAGAGCGAGGAACATCCAGACT
CTGCCCGGAATATTTAAGACAAAGGATGGGGACATTGGAGCAGTTGCGCTGGAC
TACCCAGCAGGTACCTCAGGATCTCCAATCCTAGATAAGTGTGGGAGAGTGATA
GGACTCTATGGTAATGGGGTCGTGATCAAAAATGGGAGTTACGTTAGTGCCATCA
CCCAAGGGAGGAGGGAGGAAGAGACTCCTGTTGAGTGCTTCGAGCCTTCGATGC
TGAAGAAGAAGCAGCTAACTGTCTTAGACTTGCATCCTGGAGCTGGGAAAACCA
GGAGAGTTCTTCCTGAAATAGTCCGTGAAGCCATAAAAACAAGACTCCGCACTG
TGATCTTAGCTCCAACCAGGGTTGTCGCTGCTGAAATGGAGGAAGCCCTTAGAGG
GCTTCCAGTGCGTTATATGACAACAGCAGTCAATGTCACCCATTCTGGGACAGAA
ATCGTTGACTTAATGTGCCATGCCACCTTCACTTCACGTCTACTACAGCCAATCA
GAGTCCCCAACTATAATCTGTATATTATGGATGAGGCCCACTTCACAGATCCCTC
AAGTATAGCAGCAAGAGGATACATTTCAACAAGGGTTGAGATGGGCGAGGCGGC
TGCCATCTTCATGACTGCCACGCCACCAGGAACCCGTGACGCATTCCCGGACTCC
AACTCACCAATTATGGACACCGAAGTGGAAGTCCCAGAGAGAGCCTGGAGCTCA
GGCTTTGATTGGGTGACGGATCATTCTGGAAAAACAGTTTGGTTTGTTCCAAGCG
TGAGGAATGGCAATGAGATCGCAGCTTGTCTGACAAAGGCTGGAAAACGGGTCA
TACAGCTCAGCAGAAAGACTTTTGAGACAGAGTTCCAGAAAACAAAACATCAAG
AGTGGGACTTCGTCGTGACAACTGACATTTCAGAGATGGGCGCCAACTTTAAAGC
TGACCGTGTCATAGATTCCAGGAGATGCCTAAAGCCGGTCATACTTGATGGCGAG
AGAGTCATTCTGGCTGGACCCATGCCTGTCACACATGCCAGCGCTGCCCAGAGG
AGGGGGCGCATAGGCAGGAACCCCAACAAACCTGGAGATGAGTATCTGTATGGA
GGTGGGTGCGCAGAGACTGATGAAGACCATGCACACTGGCTTGAAGCAAGAATG
CTTCTTGACAACATTTACCTCCAAGATGGCCTCATAGCCTCGCTCTATCGACCTGA
GGCCGACAAAGTAGCAGCTATTGAGGGAGAGTTCAAGCTTAGGACGGAGCAAAG
GAAGACCTTTGTGGAACTCATGAAAAGAGGAGATCTTCCTGTTTGGCTGGCCTAT
CAGGTTGCATCTGCCGGAATAACCTACACAGATAGAAGATGGTGCTTTGATGGC
ACGACCAACAACACCATAATGGAAGACAGTGTGCCGGCAGAGGTGTGGACCAGA
TACGGAGAGAAAAGAGTGCTCAAACCGAGGTGGATGGACGCCAGAGTTTGTTCA
GATCATGCGGCCCTGAAGTCATTCAAAGAGTTTGCCGCTGGGAAAAGAGGAGCG
GCCTTTGGAGTGATGGAAGCCCTGGGAACACTGCCAGGACATATGACAGAGAGA
TTCCAGGAGGCCATTGACAACCTCGCTGTGCTCATGCGGGCAGAGACTGGAAGC
AGGCCCTACAAAGCCGCGGCGGCCCAATTACCGGAGACCCTAGAGACTATCATG
CTTTTGGGGTTGCTGGGAACAGTCTCGCTGGGAATCTTTTTCGTCTTGATGCGGA
ACAAGGGCATAGGGAAGATGGGCTTTGGAATGGTGACTCTTGGGGCCAGCGCAT
GGCTTATGTGGCTCTCGGAAATTGAGCCAGCCAGAATTGCATGTGTCCTCATTGT
TGTGTTCCTATTGCTGGTGGTGCTCATACCTGAGCCAGAAAAGCAAAGATCTCCC
CAGGACAACCAAATGGCAATCATCATCATGGTAGCAGTGGGTCTTCTGGGCTTGA
TTACCGCCAATGAACTCGGATGGTTGGAGAGAACAAAGAGTGACCTAAGCCATC
TAATGGGAAGGAGAGAGGAGGGGGCAACTATAGGATTCTCAATGGACATTGACC
TGCGGCCAGCCTCAGCTTGGGCTATCTATGCTGCTCTGACAACTTTCATTACCCCA
GCCGTCCAACATGCAGTGACCACTTCATACAACAACTACTCCTTAATGGCGATGG
CCACGCAAGCTGGAGTGTTGTTCGGTATGGGTAAAGGGATGCCATTCTATGCATG
GGACTTTGGAGTCCCGCTGCTAATGATAGGTTGCTACTCACAATTAACACCCCTG
ACCCTAATAGTGGCCATCATTTTGCTCGTGGCGCACTACATGTACTTGATCCCAG
GGCTGCAGGCAGCAGCTGCGCGTGCTGCCCAGAAGAGAACGGCAGCTGGCATCA
TGAAGAACCCTGTTGTGGATGGAATAGTGGTGACTGACATTGACACAATGACAA
TTGACCCCCAAGTGGAGAAAAAGATGGGACAGGTGCTACTCATAGCAGTAGCTG
TCTCCAGCGCCATACTGTCGCGGACCGCCTGGGGGTGGGGTGAGGCTGGGGCCC
TGATCACAGCTGCAACTTCCACTTTGTGGGAGGGCTCTCCGAACAAGTACTGGAA
CTCCTCCACAGCCACCTCACTGTGTAACATTTTTAGGGGAAGCTACTTGGCTGGA
GCTTCTCTAATCTACACAGTAACAAGAAACGCTGGCTTGGTCAAGAGACGTGGG
GGTGGAACGGGAGAGACCCTGGGAGAGAAATGGAAGGCCCGCCTGAACCAGAT
GTCGGCCCTGGAGTTCTACTCCTACAAAAAGTCAGGCATCACCGAGGTGTGCAG
AGAAGAGGCCCGCCGCGCCCTCAAGGACGGTGTGGCAACGGGAGGCCACGCTGT
GTCCCGAGGAAGTGCAAAGCTGAGATGGTTGGTGGAGAGGGGATACCTGCAGCC
CTATGGAAAGGTCATTGATCTTGGATGTGGCAGAGGGGGCTGGAGTTACTATGCC
GCCACCATCCGCAAAGTTCAAGAAGTGAAAGGATACACAAAAGGAGGCCCTGGT
CATGAAGAACCCATGTTGGTGCAAAGCTATGGGTGGAACATAGTCCGTCTTAAG
AGTGGGGTGGACGTCTTTCATATGGCGGCTGAGCCGTGTGACACGTTGCTGTGTG
ATATAGGTGAGTCATCATCTAGTCCTGAAGTGGAAGAAGCACGGACGCTCAGAG
TCCTCTCCATGGTGGGGGATTGGCTTGAAAAAAGACCAGGAGCCTTTTGTATAAA
AGTGTTGTGCCCATACACCAGCACTATGATGGAAACCCTGGAGCGACTGCAGCG
TAGGTATGGGGGAGGACTGGTCAGAGTGCCACTCTCCCGCAACTCTACACATGA
GATGTACTGGGTCTCTGGAGCGAAAAGCAACACCATAAAAAGTGTGTCCACCAC
GAGCCAGCTCCTTTTGGGGCGCATGGACGGGCCCAGGAGGCCAGTGAAATATGA
AGAGGATGTGAATCTCGGCTCTGGCACGCGGGCTGTGGTAAGCTGCGCTGAAGC
TCCCAACATGAAGATCATTGGTAACCGCATTGAGAGGATCCGCAGTGAGCACGC
GGAAACGTGGTTCTTTGACGAGAACCACCCATATAGGACATGGGCTTACCATGG
AAGCTACGAGGCCCCCACACAAGGGTCAGCGTCCTCTCTAATAAACGGGGTTGT
CAGGCTCCTGTCAAAACCCTGGGATGTGGTGACTGGAGTCACAGGAATAGCCAT
GACCGACACCACACCGTATGGTCAGCAAAGAGTTTTCAAGGAAAAAGTGGACAC
TAGGGTGCCAGACCCCCAAGAAGGCACTCGTCAGGTTATGAGCATGGTCTCTTCC
TGGTTGTGGAAAGAGTTAGGCAAACACAAACGGCCACGAGTCTGTACCAAAGAA
GAGTTCATCAACAAGGTTCGTAGCAACGCAGCATTAGGGGCAATATTTGAAGAG
GAAAAAGAGTGGAAGACTGCAGTGGAAGCTGTGAACGATCCAAGGTTCTGGGCT
CTAGTGGACAAGGAAAGAGAGCACCACCTGAGAGGAGAGTGCCAGAGCTGTGT
GTACAACATGATGGGAAAAAGAGAAAAGAAACAAGGGGAATTTGGAAAGGCCA
AGGGCAGCCGCGCCATCTGGTACATGTGGCTAGGGGCTAGATTTCTAGAGTTCGA
AGCCCTTGGATTCTTGAACGAGGATCACTGGATGGGGAGAGAGAATTCAGGAGG
TGGTGTTGAAGGGCTAGGATTACAAAGACTCGGATATGTCTTAGAAGAGATGAG
TCGCATACCAGGAGGAAGGATGTATGCAGATGATACTGCTGGCTGGGACACCCG
CATCAGCAGGTTTGATCTGGAGAATGAAGCTCTAATCACCAACCAAATGGAGAA
AGGGCACAGGGCCTTGGCATTGGCCATAATCAAGTACACATACCAAAACAAAGT
GGTAAAGGTCCTTAGACCAGCTGAAAAAGGGAAGACAGTTATGGACATTATTTC
AAGACAAGACCAAAGGGGGAGCGGACAAGTTGTCACTTACGCTCTTAATACATT
TACCAACCTAGTGGTGCAGCTCATTCGGAATATGGAGGCTGAGGAAGTTCTAGA
GATGCAAGACTTGTGGCTGCTGCGGAGGTCAGAGAAAGTGACCAACTGGTTGCA
GAGCAATGGATGGGATAGGCTCAAACGAATGGCAGTCAGTGGAGATGATTGCGT
TGTGAAACCAATTGATGATAGGTTTGCACATGCTCTCAGGTTCTTGAATGATATG
GGAAAAGTTAGGAAGGACACACAAGAGTGGAAGCCCTCAACTGGATGGGACAA
CTGGGAAGAAGTTCCGTTTTGCTCCCACCACTTCAACAAGCTCCATCTCAAGGAC
GGGAGGTCCATTGTGGTTCCCTGCCGCCACCAAGATGAACTGATTGGCCGAGCTC
GCGTCTCACCGGGGGCGGGATGGAGCATCCGGGAGACTGCTTGCCTAGCAAAAT
CATATGCGCAAATGTGGCAGCTCCTTTATTTCCACAGAAGGGACCTCCGACTGAT
GGCCAATGCCATTTGTTCATCTGTGCCAGTTGACTGGGTTCCAACTGGGAGAACT
ACCTGGTCAATCCATGGAAAGGGAGAATGGATGACCACTGAAGACATGCTTGTG
GTGTGGAACAGAGTGTGGATTGAGGAGAACGACCACATGGAAGACAAGACCCC
AGTTACGAAATGGACAGACATTCCCTATTTGGGAAAAAGGGAAGACTTGTGGTG
TGGGTCTCTCATAGGGCACAGACCGCGCACCACCTGGGCTGAGAACATTAAAAA
CACAGTCAACATGATGCGTAGGATCATAGGTGATGAAGAAAAGTACGTGGACTA
CCTATCCACCCAAGTTCGCTACTTGGGCGAAGAAGGGTCCACACCTGGAGTGCTA
TAAGCACCAATCTTAGTGTTGTCAGGCCTGCTAGTCAGCCACAGCTTGGGGAAAG
CTGTGCAGCCTGTGACCCCCCCAGGAGAAGCTGGGAAACCAAGCCCATAGTCAG
GCCGAGAACGCCATGGCACGGAAGAAGCCATGCTGCCTGTGAGCCCCTCAGAGG
ACACTGAGTCAAAAAACCCCACGCGCTTGGAGGCGCAGGATGGGAAAAGAAGG
TGGCGACCTTCCCCACCCTTTAATCTGGGGCCTGAACTGGAGATCAGCTGTGGAT
CTCCAGAAGAGGGACTAGTGGTTAGAGGAGACCCCCCGGAAAACGCAAAACAG
CATATTGACGCTGGGAAAGACCAGAGACTCCATGAGTTTCCACCACGCTGGCCG
CCAGGCACAGATCGCCGAATAGCGGCGGCCGGTGTGGGGAAATCCATGGgTCTgg
gtcggcatggcatctccacctcctcgcggtccgacctgggctacttcggtaggctaagggagaagaatcgatgctgtgccttc
tagttgccagccatctgttgtttgcccctcccccgtgccttccttgaccctggaaggtgccactcccactgtcctttcctaat
aaaatgaggaaattgcatcgcattgtctgagtaggtgtcattctattctggggggtggggtggggcaggacagcaagggggag
gattgggaagacaatagcaggcatgctggggatgcggtgggctctatgggcccgggccgtcgaccaattctcatgtttgacag
cttatcatcgaatttctgccattcatccgcttattatcacttattcaggcgtagcaaccaggcgtttaagggcaccaataact
gccttaaaaaaattacgccccgccctgccactcatcgcagtactgttgtaattcattaagcattctgccgacatggaagccat
cacaaacggcatgatgaacctgaatcgccagcggcatcagcaccttgtcgccttgcgtataatatttgcccatggtgaaaacg
ggggcgaagaagttgtccatattggccacgtttaaatcaaaactggtgaaactcacccagggattggctgagacgaaaaacat
attctcaataaaccctttagggaaataggccaggttttcaccgtaacacgccacatcttgcgaatatatgtgtagaaactgcc
ggaaatcgtcgtggtattcactccagagcgatgaaaacgtttcagtttgctcatggaaaacggtgtaacaagggtgaacacta
tcccatatcaccagacaccgtctttcattgccatacgaaattccggatgagcattcatcaggcgggcaagaatgtgaataaag
gccggataaaacttgtgcttatttttctttacggtctttaaaaaggccgtaatatccagctgaacggtctggttataggtaca
ttgagcaactgactgaaatgcctcaaaatgttctttacgatgccattgggatatatcaacggtggtatatccagtgatttttt
tctccattttagcttccttagctcctgaaaatctcgataactcaaaaaatacgcccggtagtgatcttatttcattatggtga
aagttggaacctcttacgtgccgatcaacgtctcattttcgccaaaagttggcccagggcttcccggtatcaacagggacacc
aggatttatttattctgcgaagtgatcttccgtcacaggtatttattcgcgataagctcatggagcggcgtaaccgtcgcaca
ggaaggacagagaaagcgcggatctgggaagtgacggacagaacggtcaggacctggattggggaggcggttgccgccgctgc
tgctgacggtgtgacgttctctgttccggtcacaccacatacgttccgccattcctatgcgatgcacatgctgtatgccggta
taccgctgaaagttctgcaaagcctgatgggacataagtccatcagttcaacggaagtctacacgaaggtttttgcgctggat
gtggctgcccggcaccgggtgcagtttgcgatgccggagtctgatgcggttgcgatgctgaaacaattatcctgagaataaat
gccttggcctttatatggaaatgtggaactgagtggatatgctgtttttgtctgttaaacagagaagctggctgttatccact
gagaagcgaacgaaacagtcgggaaaatctcccattatcgtagagatccgcattattaatctcaggagcctgtgtagcgttta
taggaagtagtgttctgtcatgatgcctgcaagcggtaacgaaaacgatttgaatatgccttcaggaacaatagaaatcttcg
tgcggtgttacgttgaagtggagcggattatgtcagcaatggacagaacaacctaatgaacacagaaccatgatgtggtctgt
ccttttacagccagtagtgctcgccgcagtcgagcgacagggcgaagccctcggctggttgccctcgccgctgggctggcggc
cgtctatggccctgcaaacgcgccagaaacgccgtcgaagccgtgtgcgagacaccgcggccggccgccggcgttgtggatac
ctcgcggaaaacttggccctcactgacagatgaggggcggacgttgacacttgaggggccgactcacccggcgcggcgttgac
agatgaggggcaggctcgatttcggccggcgacgtggagctggccagcctcgcaaatcggcgaaaacgcctgattttacgcga
gtttcccacagatgatgtggacaagcctggggataagtgccctgcggtattgacacttgaggggcgcgactactgacagatga
ggggcgcgatccttgacacttgaggggcagagtgctgacagatgaggggcgcacctattgacatttgaggggctgtccacagg
cagaaaatccagcatttgcaagggtttccgcccgtttttcggccaccgctaacctgtcttttaacctgcttttaaaccaatat
ttataaaccttgtttttaaccagggctgcgccctgtgcgcgtgaccgcgcacgccgaaggggggtgcccccccttctcgaacc
ctcccggtcgagtgagcgaggaagcaccagggaacagcacttatatattctgcttacacacgatgcctgaaaaaacttccctt
ggggttatccacttatccacggggatatttttataattattttttttatagtttttagatcttcttttttagagcgccttgta
ggcctttatccatgctggttctagagaaggtgttgtgacaaattgccctttcagtgtgacaaatcaccctcaaatgacagtcc
tgtctgtgacaaattgcccttaaccctgtgacaaattgccctcagaagaagctgttttttcacaaagttatccctgcttattg
actcttttttatttagtgtgacaatctaaaaacttgtcacacttcacatggatctgtcatggcggaaacagcggttatcaatc
acaagaaacgtaaaaatagcccgcgaatcgtccagtcaaacgacctcactgaggcggcatatagtctctcccgggatcaaaaa
cgtatgctgtatctgttcgttgaccagatcagaaaatctgatggcaccctacaggaacatgacggtatctgcgagatccatgt
tgctaaatatgctgaaatattcggattgacctctgcggaagccagtaaggatatacggcaggcattgaagagtttcgcgggga
aggaagtggttttttatcgccctgaagaggatgccggcgatgaaaaaggctatgaatcttttccttggtttatcaaacgtgcg
cacagtccatccagagggctttacagtgtacatatcaacccatatctcattcccttctttatcgggttacagaaccggtttac
gcagtttcggcttagtgaaacaaaagaaatcaccaatccgtatgccatgcgtttatacgaatccctgtgtcagtatcgtaagc
cggatggctcaggcatcgtctctctgaaaatcgactggatcatagagcgttaccagctgcctcaaagttaccagcgtatgcct
gacttccgccgccgcttcctgcaggtctgtgttaatgagatcaacagcagaactccaatgcgcctctcatacattgagaaaaa
gaaaggccgccagacgactcatatcgtattttccttccgcgatatcacttccatgacgacaggatagtctgagggttatctgt
cacagatttgagggtggttcgtcacatttgttctgacctactgagggtaatttgtcacagttttgctgtttccttcagcctgc
atggattttctcatactttttgaactgtaatttttaaggaagccaaatttgagggcagtttgtcacagttgatttccttctct
ttcccttcgtcatgtgacctgatatcgggggttagttcgtcatcattgatgagggttgattatcacagtttattactctgaat
tggctatccgcgtgtgtacctctacctggagtttttcccacggtggatatttcttcttgcgctgagcgtaagagctatctgac
agaacagttcttctttgcttcctcgccagttcgctcgctatgctcggttacacggctgcggcgagcgctagtgataataagtg
actgaggtatgtgctcttcttatctccttttgtagtgttgctcttattttaaacaactttgcggttttttgatgactttgcga
ttttgttgttgctttgcagtaaattgcaagatttaataaaaaaacgcaaagcaatgattaaaggatgttcagaatgaaactca
tggaaacacttaaccagtgcataaacgctggtcatgaaatgacgaaggctatcgccattgcacagtttaatgatgacagcccg
gaagcgaggaaaataacccggcgctggagaataggtgaagcagcggatttagttggggtttcttctcaggctatcagagatgc
cgagaaagcagggcgactaccgcacccggatatggaaattcgaggacgggttgagcaacgtgttggttatacaattgaacaaa
ttaatcatatgcgtgatgtgtttggtacgcgattgcgacgtgctgaagacgtatttccaccggtgatcggggttgctgcccat
aaaggtggcgtttacaaaacctcagtttctgttcatcttgctcaggatctggctctgaaggggctacgtgttttgctcgtgga
aggtaacgacccccagggaacagcctcaatgtatcacggatgggtaccagatcttcatattcatgcagaagacactctcctgc
ctttctatcttggggaaaaggacgatgtcacttatgcaataaagcccacttgctggccggggcttgacattattccttcctgt
ctggctctgcaccgtattgaaactgagttaatgggcaaatttgatgaaggtaaactgcccaccgatccacacctgatgctccg
actggccattgaaactgttgctcatgactatgatgtcatagttattgacagcgcgcctaacctgggtatcggcacgattaatg
tcgtatgtgctgctgatgtgctgattgttcccacgcctgctgagttgtttgactacacctccgcactgcagtttttcgatatg
cttcgtgatctgctcaagaacgttgatcttaaagggttcgagcctgatgtacgtattttgcttaccaaatacagcaatagtaa
tggctctcagtccccgtggatggaggagcaaattcgggatgcctggggaagcatggttctaaaaaatgttgtacgtgaaacgg
atgaagttggtaaaggtcagatccggatgagaactgtttttgaacaggccattgatcaacgctcttcaactggtgcctggaga
aatgctctttctatttgggaacctgtctgcaatgaaattttcgatcgtctgattaaaccacgctgggagattagataatgaag
cgtgcgcctgttattccaaaacatacgctcaatactcaaccggttgaagatacttcgttatcgacaccagctgccccgatggt
ggattcgttaattgcgcgcgtaggagtaatggctcgcggtaatgccattactttgcctgtatgtggtcgggatgtgaagttta
ctcttgaagtgctccggggtgatagtgttgagaagacctctcgggtatggtcaggtaatgaacgtgaccaggagctgcttact
gaggacgcactggatgatctcatcccttcttttctactgactggtcaacagacaccggcgttcggtcgaagagtatctggtgt
catagaaattgccgatgggagtcgccgtcgtaaagctgctgcacttaccgaaagtgattatcgtgttctggttggcgagctgg
atgatgagcagatggctgcattatccagattgggtaacgattatcgcccaacaagtgcttatgaacgtggtcagcgttatgca
agccgattgcagaatgaatttgctggaaatatttctgcgctggctgatgcggaaaatatttcacgtaagattattacccgctg
tatcaacaccgccaaattgcctaaatcagttgttgctcttttttctcaccccggtgaactatctgcccggtcaggtgatgcac
ttcaaaaagcctttacagataaagaggaattacttaagcagcaggcatctaaccttcatgagcagaaaaaagctggggtgata
tttgaagctgaagaagttatcactcttttaacttctgtgcttaaaacgtcatctgcatcaagaactagtttaagctcacgaca
tcagtttgctcctggagcgacagtattgtataagggcgataaaatggtgcttaacctggacaggtctcgtgttccaactgagt
gtatagagaaaattgaggccattcttaaggaacttgaaaagccagcaccctgatgcgaccacgttttagtctacgtttatctg
tctttacttaatgtcctttgttacaggccagaaagcataactggcctgaatattctctctgggcccactgttccacttgtatc
gtcggtctgataatcagactgggaccacggtcccactcgtatcgtcggtctgattattagtctgggaccacggtcccactcgt
atcgtcggtctgattattagtctgggaccacggtcccactcgtatcgtcggtctgataatcagactgggaccacggtcccact
cgtatcgtcggtctgattattagtctgggaccatggtcccactcgtatcgtcggtctgattattagtctgggaccacggtccc
actcgtatcgtcggtctgattattagtctggaaccacggtcccactcgtatcgtcggtctgattattagtctgggaccacggt
cccactcgtatcgtcggtctgattattagtctgggaccacgatcccactcgtgttgtcggtctgattatcggtctgggaccac
ggtcccacttgtattgtcgatcagactatcagcgtgagactacgattccatcaatgcctgtcaagggcaagtattgacatgtc
gtcgtaacctgtagaacggagtaacctcggtgtgcggttgtatgcctgctgtggattgctgctgtgtcctgcttatccacaac
attttgcgcacggttatgtggacaaaatacctggttacccaggccgtgccggcacgttaacCGTTACATAACTTACGGTAAAT
GGCCCGCCTGGCTGACCGC
CCAACGACCCCCGCCCATTGACGTCAATAATGACGTATGTTCCCATAGTAACGCC
AATAGGGACTTTCCATTGACGTCAATGGGTGGAGTATTTACGGTAAACTGCCCAC
TTGGCAGTACATCAAGTGTATCATATGCCAAGTACGCCCCCTATTGACGTCAATG
ACGGTAAATGGCCCGCCTGGCATTATGCCCAGTACATGACCTTATGGGACTTTCC
TACTTGGCAGTACATCTACGTATTAGTCATCGCTATTACCATGGTGATGCGGTTTT
GGCAGTACATCAATGGGCGTGGATAGCGGTTTGACTCACGGGGATTTCCAAGTCT
CCACCCCATTGACGTCAATGGGAGTTTGTTTTGGCACCAAAATCAACGGGACTTT
CCAAAATGTCGTAACAACTCCGCCCCATTGACGCAAATGGGCGGTAGGCGTGTA
CGGTGGGAGGTCTATATAAGCAGAGCTCTCTGGCTAAC
SV40 ZIKV FL sequence:
SEQ ID NO: 7
agttgttgatctgtgtgaatcagactgcgacagttcgagtttgaagcgaaagctagcaacagtatcaacaggttttattttgg
atttggaaacgagagtttctggtcatgaaaaacccaaagaagaaatccggaggattccggattgtcaatatgctaaaacgcgg
agtagcccgtgtgagcccctttgggggcttgaagaggctgccagccggacttctgctgggtcatgggcccatcaggatggtct
tggcgattctagcctttttgagattcacggcaatcaagccatcactgggtctcatcaatagatggggttcagtggggaaaaaa
gaggctatggaaataataaagaagtttaagaaagatctggctgccatgctgagaataatcaatgctaggaaggagaagaagag
acgaggcacagatactagtgtcggaattgttggcctcctgctgaccacagccatggcagtggaggtcactagacgtgggaatg
catactatatgtacttggacagaagcgatgctggggaggccatatcttttccaaccacaatggggatgaataagtgttatata
cagatcatggatcttggacacatgtgtgatgccaccatgagctatgaatgccctatgctggatgagggggtagaaccagatga
cgtcgattgttggtgcaacacgacgtcaacttgggttgtgtacggaacctgccaccacaaaaaaggtgaagcacggagatcta
gaagagctgtgacgctcccctcccattccactaggaagctgcaaacgcggtcgcagacctggttggaatcaagagaatacaca
aagcacctgattagagtcgaaaattggatattcaggaaccctggcttcgcgttagcagcagctgccatcgcttggcttttggg
aagctcaacgagccaaaaagtcatatacttggtcatgatactgctgattgccccggcatacagcatcaggtgcataggagtca
gcaatagggactttgtggaaggtatgtcaggtgggacttgggttgatgttgtcttggaacatggaggttgtgttaccgtaatg
gcacaggacaaaccgactgtcgacatagagctggttacaacaacagtcagcaacatggcggaggtaagatcctactgctatga
ggcatcaatatcggacatggcttcggacagccgctgcccaacacaaggtgaagcctaccttgacaagcaatcagacactcaat
atgtctgcaaaagaacgttagtggacagaggctggggaaatggatgtggactttttggcaaagggagcctggtgacatgcgct
aagtttgcttgctctaagaaaatgaccgggaagagcatccagccagagaatctggagtaccggataatgctgtcagttcatgg
ctcccagcacagtgggatgatcgttaatgatacaggacatgaaactgatgagaatagagcgaaggttgagataacgcccaatt
caccaagagccgaagccaccctggggggttttggaagcctaggacttgattgtgaaccgaggacaggccttgacttttcagat
ttgtattacttgactatgaataacaagcactggttggttcacaaggagtggttccacgacattccattaccttggcacgctgg
ggcagacaccggaactccacactggaacaacaaagaagcactggtagagttcaaggacgcacatgccaaaaggcagactgtcg
tggttctagggagtcaagaaggagcagttcacacggcccttgctggagctctggaggctgagatggatggtgcaaagggaagg
ctgtcctctggccacttgaaatgtcgcctgaaaatggacaaacttagattgaagggcgtgtcatactccttgtgtaccgcagc
gttcacattcactaagatcccggctgaaacactgcacgggacagtcacagtggaggtacagtacgcagggacagatggacctt
gcaaggttccagctcagatggcggtggacatgcaaactctgaccccagttgggaggttgataaccgctaaccctgtaatcact
gaaagcactgagaactccaagatgatgctggaactggatccaccatttggggactcttacattgtcataggagtcggggaaaa
gaagatcacccaccactggcacaggagtggcagcaccattggaaaagcatttgaagccactgtgagaggtgccaagagaatgg
cagtcttgggagacacagcctgggactttggatcagttgggggtgctctcaactcactgggcaagggcatccatcaaattttt
ggagcagctttcaaatcattgtttggaggaatgtcctggttctcacaaattctcattggaacgttgctggtgtggttgggtct
gaatacaaagaatggatctatttcccttatgtgcttggccttagggggagtgttgatcttcttatccacagccgtctctgctg
atgtggggtgctcggtggacttctcaaagaaggaaacgagatgcggtacaggggtgttcgtctataacgacgttgaagcttgg
agggacaggtacaagtaccatcctgactcccctcgtagattggcagcagcagtcaagcaagcctgggaagatgggatctgtgg
gatctcctctgtttcaagaatggaaaacatcatgtggagatcagtagaaggggagctcaacgcaatcctggaagagaatggag
ttcaactgacggtcgttgtgggatctgtaaaaaaccccatgtggagaggtccacagagattgcccgtgcctgtgaacgagctg
ccccatggctggaaggcttgggggaaatcgtacttcgtcagggcagcaaagacaaataacagctttgtcgtggatggtgacac
actgaaggaatgcccactcaaacatagagcatggaacagctttcttgtggaggatcatgggttcggggtatttcacactagtg
tctggctcaaggttagagaagattattcactcgagtgtgatccagccgtcattggaacagccgctaagggaaaggaggctgtg
cacagtgatctaggctactggattgagagtgagaagaacgacacatggaggctgaagagggcccacctgatcgagatgaaaac
atgtgaatggccaaagtcccacacattgtggacagatggaatagaagaaagtgatctgatcatacccaagtctttagctgggc
cactcagccatcacaacaccagagagggctacaggacccaaatgaaagggccatggcatagtgaagagcttgaaattcggttt
gaggaatgcccaggcactaaggtccacgtggaggaaacatgtggaacaagaggaccatctctgagatcaaccactgcaagcgg
aagggtgatcgaggaatggtgctgcagggagtgcacaatgcccccactgtcgttccgggctaaagatggttgttggtatggaa
tggagataaggcccaggaaagaaccagaaagtaacttagtaaggtcaatggtgactgcaggatcaactgatcacatggatcac
ttctcccttggagtgcttgtgattctgctcatggtacaggaagggctaaagaagagaatgaccacaaagatcatcataagcac
atcaatggcagtgctggtagctatgatcctgggaggattttcaatgagtgacctggctaagcttgcaattttgatgggtgcca
ccttcgcggaaatgaacactggaggagatgttgctcatctggcgctgatagcggcattcaaagtcagacctgcgttgctggta
tctttcattttcagagctaattggacaccccgtgagagcatgctgctggccttggcctcgtgtcttctgcaaactgcgatctc
cgccttggaaggcgacctgatggttcccatcaatggttttgctttggcctggttggcaatacgagcgatggttgttccacgca
ctgacaacatcaccttggcaatcctggctgctctgacaccactggcccggggcacactgcttgtggcgtggagagcaggcctt
gctacttgcggggggttcatgctcctttctctgaaggggaaaggcagtgtgaagaagaacttaccatttgtcatggccctggg
actaaccgctgtgaggctggtcgaccccatcaacgtggtgggactgctgttgctcacaaggagtgggaagcggagctggcccc
ctagtgaagtactcacagctgttggcctgatatgcgcattggctggagggttcgccaaggcggatatagagatggctgggccc
atggccgcggtcggtctgctaattgtcagttacgtggtctcaggaaagagtgtggacatgtacattgaaagagcaggtgacat
cacatgggaaaaagatgcggaagtcactggaaacagtccccggctcgatgtggcactagatgagagtggtgatttctccctag
tggaggatgatggtccccccatgagagagatcatactcaaagtggtcctgatggccatctgtggcatgaacccaatagccata
ccctttgcagctggagcgtggtacgtgtatgtgaagactggaaaaaggagtggtgctctatgggatgtgcctgctcccaagga
agtaaaaaagggggagaccacagatggagtgtacagagtaatgactcgtagactgctaggttcaacacaagttggagtgggag
tcatgcaagagggggtcttccacactatgtggcacgtcacaaaaggatccgcgctgagaagcggtgaagggagacttgatcca
tactggggagatgtcaagcaggatctggtgtcatactgtggtccatggaagctagatgccgcctgggacgggcacagcgaggt
gcagctcttggccgtgccccccggagagagagcgaggaacatccagactctgcccggaatatttaagacaaaggatggggaca
ttggagcagttgcgctggactacccagcaggtacctcaggatctccaatcctagataagtgtgggagagtgataggactctat
ggtaatggggtcgtgatcaaaaatgggagttacgttagtgccatcacccaagggaggagggaggaagagactcctgttgagtg
cttcgagccttcgatgctgaagaagaagcagctaactgtcttagacttgcatcctggagctgggaaaaccaggagagttcttc
ctgaaatagtccgtgaagccataaaaacaagactccgcactgtgatcttagctccaaccagggttgtcgctgctgaaatggag
gaagcccttagagggcttccagtgcgttatatgacaacagcagtcaatgtcacccattctgggacagaaatcgttgacttaat
gtgccatgccaccttcacttcacgtctactacagccaatcagagtccccaactataatctgtatattatggatgaggcccact
tcacagatccctcaagtatagcagcaagaggatacatttcaacaagggttgagatgggcgaggcggctgccatcttcatgact
gccacgccaccaggaacccgtgacgcattcccggactccaactcaccaattatggacaccgaagtggaagtcccagagagagc
ctggagctcaggctttgattgggtgacggatcattctggaaaaacagtttggtttgttccaagcgtgaggaatggcaatgaga
tcgcagcttgtctgacaaaggctggaaaacgggtcatacagctcagcagaaagacttttgagacagagttccagaaaacaaaa
catcaagagtgggacttcgtcgtgacaactgacatttcagagatgggcgccaactttaaagctgaccgtgtcatagattccag
gagatgcctaaagccggtcatacttgatggcgagagagtcattctggctggacccatgcctgtcacacatgccagcgctgccc
agaggagggggcgcataggcaggaaccccaacaaacctggagatgagtatctgtatggaggtgggtgcgcagagactgatgaa
gaccatgcacactggcttgaagcaagaatgcttcttgacaacatttacctccaagatggcctcatagcctcgctctatcgacc
tgaggccgacaaagtagcagctattgagggagagttcaagcttaggacggagcaaaggaagacctttgtggaactcatgaaaa
gaggagatcttcctgtttggctggcctatcaggttgcatctgccggaataacctacacagatagaagatggtgctttgatggc
acgaccaacaacaccataatggaagacagtgtgccggcagaggtgtggaccagatacggagagaaaagagtgctcaaaccgag
gtggatggacgccagagtttgttcagatcatgcggccctgaagtcattcaaagagtttgccgctgggaaaagaggagcggcct
ttggagtgatggaagccctgggaacactgccaggacatatgacagagagattccaggaggccattgacaacctcgctgtgctc
atgcgggcagagactggaagcaggccctacaaagccgcggcggcccaattaccggagaccctagagactatcatgcttttggg
gttgctgggaacagtctcgctgggaatctttttcgtcttgatgcggaacaagggcatagggaagatgggctttggaatggtga
ctcttggggccagcgcatggcttatgtggctctcggaaattgagccagccagaattgcatgtgtcctcattgttgtgttccta
ttgctggtggtgctcatacctgagccagaaaagcaaagatctccccaggacaaccaaatggcaatcatcatcatggtagcagt
gggtcttctgggcttgattaccgccaatgaactcggatggttggagagaacaaagagtgacctaagccatctaatgggaagga
gagaggagggggcaactataggattctcaatggacattgacctgcggccagcctcagcttgggctatctatgctgctctgaca
actttcattaccccagccgtccaacatgcagtgaccacttcatacaacaactactccttaatggcgatggccacgcaagctgg
agtgttgttcggtatgggtaaagggatgccattctatgcatgggactttggagtcccgctgctaatgataggttgctactcac
aattaacacccctgaccctaatagtggccatcattttgctcgtggcgcactacatgtacttgatcccagggctgcaggcagca
gctgcgcgtgctgcccagaagagaacggcagctggcatcatgaagaaccctgttgtggatggaatagtggtgactgacattga
cacaatgacaattgacccccaagtggagaaaaagatgggacaggtgctactcatagcagtagctgtctccagcgccatactgt
cgcggaccgcctgggggtggggtgaggctggggccctgatcacagctgcaacttccactttgtgggagggctctccgaacaag
tactggaactcctccacagccacctcactgtgtaacatttttaggggaagctacttggctggagcttctctaatctacacagt
aacaagaaacgctggcttggtcaagagacgtgggggtggaacgggagagaccctgggagagaaatggaaggcccgcctgaacc
agatgtcggccctggagttctactcctacaaaaagtcaggcatcaccgaggtgtgcagagaagaggcccgccgcgccctcaag
gacggtgtggcaacgggaggccacgctgtgtcccgaggaagtgcaaagctgagatggttggtggagaggggatacctgcagcc
acttggaaaggtcattgatcttggatgtggcagagggggctggagttactatgccgccaccatccgcaaagttcaagaagtga
aaggatacacaaaaggaggccctggtcatgaagaacccatgttggtgcaaagctatgggtggaacatagtccgtcttaagagt
ggggtggacgtctttcatatggcggctgagccgtgtgacacgttgctgtgtgatataggtgagtcatcatctagtcctgaagt
ggaagaagcacggacgctcagagtcctctccatggtgggggattggcttgaaaaaagaccaggagccttttgtataaaagtgt
tgtgcccatacaccagcactatgatggaaaccctggagcgactgcagcgtaggtatgggggaggactggtcagagtgccactc
tcccgcaactctacacatgagatgtactgggtctctggagcgaaaagcaacaccataaaaagtgtgtccaccacgagccagct
ccttttggggcgcatggacgggcccaggaggccagtgaaatatgaagaggatgtgaatctcggctctggcacgcgggctgtgg
taagctgcgctgaagctcccaacatgaagatcattggtaaccgcattgagaggatccgcagtgagcacgcggaaacgtggttc
tttgacgagaaccacccatataggacatgggcttaccatggaagctacgaggcccccacacaagggtcagcgtcctctctaat
aaacggggttgtcaggctcctgtcaaaaccctgggatgtggtgactggagtcacaggaatagccatgaccgacaccacaccgt
atggtcagcaaagagttttcaaggaaaaagtggacactagggtgccagacccccaagaaggcactcgtcaggttatgagcatg
gtctcttcctggttgtggaaagagttaggcaaacacaaacggccacgagtctgtaccaaagaagagttcatcaacaaggttcg
tagcaacgcagcattaggggcaatatttgaagaggaaaaagagtggaagactgcagtggaagctgtgaacgatccaaggttct
gggctctagtggacaaggaaagagagcaccacctgagaggagagtgccagagctgtgtgtacaacatgatgggaaaaagagaa
aagaaacaaggggaatttggaaaggccaagggcagccgcgccatctggtacatgtggctaggggctagatttctagagttcga
agcccttggattcttgaacgaggatcactggatggggagagagaattcaggaggtggtgttgaagggctaggattacaaagac
tcggatatgtcttagaagagatgagtcgcataccaggaggaaggatgtatgcagatgatactgctggctgggacacccgcatc
agcaggtttgatctggagaatgaagctctaatcaccaaccaaatggagaaagggcacagggccttggcattggccataatcaa
gtacacataccaaaacaaagtggtaaaggtccttagaccagctgaaaaagggaagacagttatggacattatttcaagacaag
accaaagggggagcggacaagttgtcacttacgctcttaatacatttaccaacctagtggtgcagctcattcggaatatggag
gctgaggaagttctagagatgcaagacttgtggctgctgcggaggtcagagaaagtgaccaactggttgcagagcaatggatg
ggataggctcaaacgaatggcagtcagtggagatgattgcgttgtgaaaccaattgatgataggtttgcacatgctctcaggt
tcttgaatgatatgggaaaagttaggaaggacacacaagagtggaagccctcaactggatgggacaactgggaagaagttccg
ttttgctcccaccacttcaacaagctccatctcaaggacgggaggtccattgtggttccctgccgccaccaagatgaactgat
tggccgagctcgcgtctcaccgggggcgggatggagcatccgggagactgcttgcctagcaaaatcatatgcgcaaatgtggc
agctcctttatttccacagaagggacctccgactgatggccaatgccatttgttcatctgtgccagttgactgggttccaact
gggagaactacctggtcaatccatggaaagggagaatggatgaccactgaagacatgcttgtggtgtggaacagagtgtggat
tgaggagaacgaccacatggaagacaagaccccagttacgaaatggacagacattccctatttgggaaaaagggaagacttgt
ggtgtgggtctctcatagggcacagaccgcgcaccacctgggctgagaacattaaaaacacagtcaacatgatgcgtaggatc
ataggtgatgaagaaaagtacgtggactacctatccacccaagttcgctacttgggcgaagaagggtccacacctggagtgct
ataagcaccaatcttagtgttgtcaggcctgctagtcagccacagcttggggaaagctgtgcagcctgtgacccccccaggag
aagctgggaaaccaagcccatagtcaggccgagaacgccatggcacggaagaagccatgctgcctgtgagcccctcagaggac
actgagtcaaaaaaccccacgcgcttggaggcgcaggatgggaaaagaaggtggcgaccttccccaccctttaatctggggcc
tgaactggagatcagctgtggatctccagaagagggactagtggttagaggagaccccccggaaaacgcaaaacagcatattg
acgctgggaaagaccagagactccatgagtttccaccacgctggccgccaggcacagatcgccgaatagcggcggccggtgtg
gggaaatccatgggtctgggtcggcatggcatctccacctcctcgcggtccgacctgggctacttcggtaggctaagggagaa
gaatcgatgaacttgtttattgcagcttataatggttacaaataaagcaatagcatcacaaatttcacaaataaagcattttt
ttcactgcattctagttgtggtttgtccaaactcatcaatgtatcttatgcccgggccgtcgaccaattctcatgtttgacag
cttatcatcgaatttctgccattcatccgcttattatcacttattcaggcgtagcaaccaggcgtttaagggcaccaataact
gccttaaaaaaattacgccccgccctgccactcatcgcagtactgttgtaattcattaagcattctgccgacatggaagccat
cacaaacggcatgatgaacctgaatcgccagcggcatcagcaccttgtcgccttgcgtataatatttgcccatggtgaaaacg
ggggcgaagaagttgtccatattggccacgtttaaatcaaaactggtgaaactcacccagggattggctgagacgaaaaacat
attctcaataaaccctttagggaaataggccaggttttcaccgtaacacgccacatcttgcgaatatatgtgtagaaactgcc
ggaaatcgtcgtggtattcactccagagcgatgaaaacgtttcagtttgctcatggaaaacggtgtaacaagggtgaacacta
tcccatatcaccagctcaccgtctttcattgccatacgaaattccggatgagcattcatcaggcgggcaagaatgtgaataaa
ggccggataaaacttgtgcttatttttctttacggtctttaaaaaggccgtaatatccagctgaacggtctggttataggtac
attgagcaactgactgaaatgcctcaaaatgttctttacgatgccattgggatatatcaacggtggtatatccagtgattttt
ttctccattttagcttccttagctcctgaaaatctcgataactcaaaaaatacgcccggtagtgatcttatttcattatggtg
aaagttggaacctcttacgtgccgatcaacgtctcattttcgccaaaagttggcccagggcttcccggtatcaacagggacac
caggatttatttattctgcgaagtgatcttccgtcacaggtatttattcgcgataagctcatggagcggcgtaaccgtcgcac
aggaaggacagagaaagcgcggatctgggaagtgacggacagaacggtcaggacctggattggggaggcggttgccgccgctg
ctgctgacggtgtgacgttctctgttccggtcacaccacatacgttccgccattcctatgcgatgcacatgctgtatgccggt
ataccgctgaaagttctgcaaagcctgatgggacataagtccatcagttcaacggaagtctacacgaaggtttttgcgctgga
tgtggctgcccggcaccgggtgcagtttgcgatgccggagtctgatgcggttgcgatgctgaaacaattatcctgagaataaa
tgccttggcctttatatggaaatgtggaactgagtggatatgctgtttttgtctgttaaacagagaagctggctgttatccac
tgagaagcgaacgaaacagtcgggaaaatctcccattatcgtagagatccgcattattaatctcaggagcctgtgtagcgttt
ataggaagtagtgttctgtcatgatgcctgcaagcggtaacgaaaacgatttgaatatgccttcaggaacaatagaaatcttc
gtgcggtgttacgttgaagtggagcggattatgtcagcaatggacagaacaacctaatgaacacagaaccatgatgtggtctg
tccttttacagccagtagtgctcgccgcagtcgagcgacagggcgaagccctcggctggttgccctcgccgctgggctggcgg
ccgtctatggccctgcaaacgcgccagaaacgccgtcgaagccgtgtgcgagacaccgcggccggccgccggcgttgtggata
cctcgcggaaaacttggccctcactgacagatgaggggcggacgttgacacttgaggggccgactcacccggcgcggcgttga
cagatgaggggcaggctcgatttcggccggcgacgtggagctggccagcctcgcaaatcggcgaaaacgcctgattttacgcg
agtttcccacagatgatgtggacaagcctggggataagtgccctgcggtattgacacttgaggggcgcgactactgacagatg
aggggcgcgatccttgacacttgaggggcagagtgctgacagatgaggggcgcacctattgacatttgaggggctgtccacag
gcagaaaatccagcatttgcaagggtttccgcccgtttttcggccaccgctaacctgtcttttaacctgcttttaaaccaata
tttataaaccttgtttttaaccagggctgcgccctgtgcgcgtgaccgcgcacgccgaaggggggtgcccccccttctcgaac
cctcccggtcgagtgagcgaggaagcaccagggaacagcacttatatattctgcttacacacgatgcctgaaaaaacttccct
tggggttatccacttatccacggggatatttttataattattttttttatagtttttagatcttcttttttagagcgccttgt
aggcctttatccatgctggttctagagaaggtgttgtgacaaattgccctttcagtgtgacaaatcaccctcaaatgacagtc
ctgtctgtgacaaattgcccttaaccctgtgacaaattgccctcagaagaagctgttttttcacaaagttatccctgcttatt
gactcttttttatttagtgtgacaatctaaaaacttgtcacacttcacatggatctgtcatggcggaaacagcggttatcaat
cacaagaaacgtaaaaatagcccgcgaatcgtccagtcaaacgacctcactgaggcggcatatagtctctcccgggatcaaaa
acgtatgctgtatctgttcgttgaccagatcagaaaatctgatggcaccctacaggaacatgacggtatctgcgagatccatg
ttgctaaatatgctgaaatattcggattgacctctgcggaagccagtaaggatatacggcaggcattgaagagtttcgcgggg
aaggaagtggttttttatcgccctgaagaggatgccggcgatgaaaaaggctatgaatcttttccttggtttatcaaacgtgc
gcacagtccatccagagggctttacagtgtacatatcaacccatatctcattcccttctttatcgggttacagaaccggttta
cgcagtttcggcttagtgaaacaaaagaaatcaccaatccgtatgccatgcgtttatacgaatccctgtgtcagtatcgtaag
ccggatggctcaggcatcgtctctctgaaaatcgactggatcatagagcgttaccagctgcctcaaagttaccagcgtatgcc
tgacttccgccgccgcttcctgcaggtctgtgttaatgagatcaacagcagaactccaatgcgcctctcatacattgagaaaa
agaaaggccgccagacgactcatatcgtattttccttccgcgatatcacttccatgacgacaggatagtctgagggttatctg
tcacagatttgagggtggttcgtcacatttgttctgacctactgagggtaatttgtcacagttttgctgtttccttcagcctg
catggattttctcatactattgaactgtaatttttaaggaagccaaatttgagggcagtttgtcacagttgatttccttctct
ttcccttcgtcatgtgacctgatatcgggggttagttcgtcatcattgatgagggttgattatcacagtttattactctgaat
tggctatccgcgtgtgtacctctacctggagtttttcccacggtggatatttcttcttgcgctgagcgtaagagctatctgac
agaacagttcttctttgcttcctcgccagttcgctcgctatgctcggttacacggctgcggcgagcgctagtgataataagtg
actgaggtatgtgctcttcttatctccttttgtagtgttgctcttattttaaacaactttgcggttttttgatgactttgcga
ttttgttgttgctttgcagtaaattgcaagatttaataaaaaaacgcaaagcaatgattaaaggatgttcagaatgaaactca
tggaaacacttaaccagtgcataaacgctggtcatgaaatgacgaaggctatcgccattgcacagtttaatgatgacagcccg
gaagcgaggaaaataacccggcgctggagaataggtgaagcagcggatttagttggggtttcttctcaggctatcagagatgc
cgagaaagcagggcgactaccgcacccggatatggaaattcgaggacgggttgagcaacgtgttggttatacaattgaacaaa
ttaatcatatgcgtgatgtgtttggtacgcgattgcgacgtgctgaagacgtatttccaccggtgatcggggttgctgcccat
aaaggtggcgtttacaaaacctcagtttctgttcatcttgctcaggatctggctctgaaggggctacgtgttttgctcgtgga
aggtaacgacccccagggaacagcctcaatgtatcacggatgggtaccagatcttcatattcatgcagaagacactctcctgc
ctttctatcttggggaaaaggacgatgtcacttatgcaataaagcccacttgctggccggggcttgacattattccttcctgt
ctggctctgcaccgtattgaaactgagttaatgggcaaatttgatgaaggtaaactgcccaccgatccacacctgatgctccg
actggccattgaaactgttgctcatgactatgatgtcatagttattgacagcgcgcctaacctgggtatcggcacgattaatg
tcgtatgtgctgctgatgtgctgattgttcccacgcctgctgagttgtttgactacacctccgcactgcagtttttcgatatg
cttcgtgatctgctcaagaacgttgatcttaaagggttcgagcctgatgtacgtattttgcttaccaaatacagcaatagtaa
tggctctcagtccccgtggatggaggagcaaattcgggatgcctggggaagcatggttctaaaaaatgttgtacgtgaaacgg
atgaagttggtaaaggtcagatccggatgagaactgtttttgaacaggccattgatcaacgctcttcaactggtgcctggaga
aatgctctttctatttgggaacctgtctgcaatgaaattttcgatcgtctgattaaaccacgctgggagattagataatgaag
cgtgcgcctgttattccaaaacatacgctcaatactcaaccggttgaagatacttcgttatcgacaccagctgccccgatggt
ggattcgttaattgcgcgcgtaggagtaatggctcgcggtaatgccattactttgcctgtatgtggtcgggatgtgaagttta
ctcttgaagtgctccggggtgatagtgttgagaagacctctcgggtatggtcaggtaatgaacgtgaccaggagctgcttact
gaggacgcactggatgatctcatcccttcttttctactgactggtcaacagacaccggcgttcggtcgaagagtatctggtgt
catagaaattgccgatgggagtcgccgtcgtaaagctgctgcacttaccgaaagtgattatcgtgttctggttggcgagctgg
atgatgagcagatggctgcattatccagattgggtaacgattatcgcccaacaagtgcttatgaacgtggtcagcgttatgca
agccgattgcagaatgaatttgctggaaatatttctgcgctggctgatgcggaaaatatttcacgtaagattattacccgctg
tatcaacaccgccaaattgcctaaatcagttgttgctcttttttctcaccccggtgaactatctgcccggtcaggtgatgcac
ttcaaaaagcctttacagataaagaggaattacttaagcagcaggcatctaaccttcatgagcagaaaaaagctggggtgata
tttgaagctgaagaagttatcactcttttaacttctgtgataaaacgtcatctgcatcaagaactagtttaagctcacgacat
cagtttgctcctggagcgacagtattgtataagggcgataaaatggtgcttaacctggacaggtctcgtgttccaactgagtg
tatagagaaaattgaggccattcttaaggaacttgaaaagccagcaccctgatgcgaccacgttttagtctacgtttatctgt
ctttacttaatgtcctttgttacaggccagaaagcataactggcctgaatattctctctgggcccactgttccacttgtatcg
tcggtctgataatcagactgggaccacggtcccactcgtatcgtcggtctgattattagtctgggaccacggtcccactcgta
tcgtcggtctgattattagtctgggaccacggtcccactcgtatcgtcggtctgataatcagactgggaccacggtcccactc
gtatcgtcggtctgattattagtctgggaccatggtcccactcgtatcgtcggtctgattattagtctgggaccacggtccca
ctcgtatcgtcggtctgattattagtctggaaccacggtcccactcgtatcgtcggtctgattattagtctgggaccacggtc
ccactcgtatcgtcggtctgattattagtctgggaccacgatcccactcgtgttgtcggtctgattatcggtctgggaccacg
gtcccacttgtattgtcgatcagactatcagcgtgagactacgattccatcaatgcctgtcaagggcaagtattgacatgtcg
tcgtaacctgtagaacggagtaacctcggtgtgcggttgtatgcctgctgtggattgctgctgtgtcctgcttatccacaaca
ttttgcgcacggttatgtggacaaaatacctggttacccaggccgtgccggcacgttaactgtgtcagttagggtgtggaaag
tccccaggctccccagcaggcagaagtatgcaaagcatgcatctcaattagtcagcaaccaggtgtggaaagtccccaggctc
cccagcaggcagaagtatgcaaagcatgcatctcaattagtcagcaaccatagtcccgcccctaactccgcccatcccgcccc
taactccgcccagttccgcccattctccgccccatggctgactaattttttttatttatgcagaggccgaggccgcctc
3′ UTR Zika 10 Nucleotide Deletion
SEQ ID NO: 8
CCAGAAGAGG
3′ UTR Zika 20 Nucleotide Deletion
SEQ ID NO: 9
CTGTGGATCTCCAGAAGAGG

The contents of the following references and all other references which are cited in this application are incorporated by reference in their entirety.

### REFERENCES

1 Costello, A. et al. Defining the syndrome associated with congenital Zika virus infection. Bull World Health Organ 94, 406-406A, doi: 10.2471/BLT.16.176990 (2016).
2 Weaver, S. C. et al. Zika virus: History, emergence, biology, and prospects for control. Antiviral Res 130, 69-80, doi: 10.1016/j.antiviral.2016.03.010 (2016).
3 Shan, C. et al. Zika Virus: Diagnosis, Therapeutics, and Vaccine ACS Infectious Diseases 2, 170-172 (2016).
4 Dawes, B. E. et al. Research and development of Zika virus vaccines. NPJ Vaccines 1, 16007, doi: 10.1038/npjvaccines.2016.7 (2016).
5 Barrett, A. D. T. Zika vaccine candidates progress through nonclinical development and enter clinical trials. Npj Vaccines 1, 16023, doi: 10.1038/npjvaccines.2016.23 (2016).
6 Larocca, R. A. et al. Vaccine protection against Zika virus from Brazil. Nature 536, 474-478, doi: 10.1038/nature 18952 (2016).
7 Dowd, K. A. et al. Rapid development of a DNA vaccine for Zika virus. Science 354, 237-240, doi: 10.1126/science.aai9137 (2016).
8 Abbink, P. et al. Protective efficacy of multiple vaccine platforms against Zika virus challenge in rhesus monkeys. Science 353, 1129-1132, doi: 10.1126/science.aah6157 (2016).
9 Whitehead, S. S. Development of TV003/TV005, a single dose, highly immunogenic live attenuated dengue vaccine; what makes this vaccine different from the Sanofi-Pasteur CYD vaccine? Expert review of vaccines 15, 509-517, doi: 10.1586/14760584.2016.1115727 (2016).
10 Shan, C. et al. An Infectious cDNA Clone of Zika Virus to Study Viral Virulence, Mosquito Transmission, and Antiviral Inhibitors. Cell Host Microbe 19, 891-900, doi: 10.1016/j.chom.2016.05.004 (2016).
11 Xie, X. et al. Zika Virus Replicons for Drug Discovery. EBioMedicine 12, 156-160, doi: 10.1016/j.ebiom.2016.09.013 (2016).
12 Lo, L., Tilgner, M., Bernard, K. & Shi, P.-Y. Functional analysis of mosquito-borne flavivirus conserved sequence elements within 3′ untranslated region of West Nile virus using a reporting replicon that differentiates between viral translation and RNA replication. J. Virol. 77, 10004-10014 (2003).
13 Pijlman, G. P. et al. A highly structured, nuclease-resistant, noncoding RNA produced by flavivirus es is required for pathogenicity. Cell Host Microbe 4, 579-591, doi: 10.1016/j.chom.2008.10.007 (2008).
14 Manokaran, G. et al. Dengue subgenomic RNA binds TRIM25 to inhibit interferon expression for epidemiological fitness. Science 350, 217-221, doi: 10.1126/science.aab3369 (2015).
15 Acceptability of cell substrates for production of biologicals. Report of a WHO Study Group on Biologicals. World Health Organ Tech Rep Ser 747, 1-29 (1987).
16 Rossi, S. L. et al. Characterization of a Novel Murine Model to Study Zika Virus. Am J Trop Med Hyg 94, 1362-1369, doi: 10.4269/ajtmh.16-0111 (2016).
17 Govero, J. et al. Zika virus infection damages the testes in mice. Nature 540, 438-442, doi: 10.1038/nature20556 (2016).
18 Ma, W. et al. Zika Virus Causes Testis Damage and Leads to Male Infertility in Mice. Cell 167, 1511-1524 e1510, doi: 10.1016/j.cell.2016.11.016 (2016).
19 Guerbois, M. et al. Outbreak of Zika Virus Infection, Chiapas State, Mexico, 2015, and First Confirmed Transmission by Aedes aegypti Mosquitoes in the Americas. J Infect Dis, doi: 10.1093/infdis/jiw302 (2016).
20 Ferreira-de-Brito, A. et al. First detection of natural infection of Aedes aegypti with Zika virus in Brazil and throughout South America. Mem Inst Oswaldo Cruz 111, 655-658, doi: 10.1590/0074-02760160332 (2016).
21 Yun, S. I. et al. Comparison of the live-attenuated Japanese Encephalitis vaccine SA14-14-2 strain with Its pre-attenuated virulent parent SA14 strain: Similarities and differences in vitro and in vivo. J Gen Virol, doi: 10.1099/jgv.0.000574 (2016).
22 Barrett, A. D. & Gould, E. A. Comparison of neurovirulence of different strains of yellow fever virus in mice. J Gen Virol 67 (Pt 4), 631-637, doi: 10.1099/0022-1317-67-4-631 (1986).
23 Khromykh, A. A., Sedlak, P. L. & Westaway, E. G. Trans-Complementation analysis of the flavivirus Kunjin ns5 gene reveals an essential role for translation of its N-terminal half in RNA replication. J. Virol. 73, 9247-9255 (1999).
24 Khromykh, A., Kenney, M. & Westaway, E. trans-Complementation of flavivirus RNA polymerase gene NS5 by using Kunjin virus replicon-expressing BHK cells. J. Virol. 72, 7270-7279 (1998).
25 Qing, M., Liu, W., Yuan, Z., Gu, F. & Shi, P. Y. A high-throughput assay using dengue-1 virus-like particles for drug discovery. Antiviral Res 86, 163-171 (2010).
26 Xie, X., Zou, J., Puttikhunt, C., Yuan, Z. & Shi, P. Y. Two distinct sets of NS2A molecules are responsible for dengue virus RNA synthesis and virion assembly. J Virol 89, 1298-1313, doi: 10.1128/JVI.02882-14 (2015).
27 Pugachev, K. et al. High fidelity of yellow fever virus RNA polymerase. J. Virol. 78, 1032-1038 (2004).
28 Xie, X. et al. Understanding Zika Virus Stability and Developing a Chimeric Vaccine through Functional Analysis. MBio 8, doi: 10.1128/mBio.02134-16 (2017).
29 Yauch, L. E. et al. A protective role for dengue virus-specific CD8⁺ T cells. J Immunol 182, 4865-4873, doi: 10.4049/jimmunol.0801974 (2009).
30 Weiskopf, D. et al. Comprehensive analysis of dengue virus-specific responses supports an HLA-linked protective role for CD8⁺ T cells. Proc Natl Acad Sci U S A 110, E2046-2053, doi: 10.1073/pnas.1305227110 (2013).
31 Shi, P. Y., Tilgner, M., Lo, M. K., Kent, K. A. & Bernard, K. A. Infectious cDNA clone of the epidemic West Nile virus from New York City. J. Virol. 76, 5847-5856 (2002).
32 Hansen, D. A., Esakky, P., Drury, A., Lamb, L. & Moley, K. H. The aryl hydrocarbon receptor is important for proper seminiferous tubule architecture and sperm development in mice. Biol Reprod 90, 8, doi: 10.1095/biolreprod.113.108845 (2014).
33 Ye, Q. et al. Genomic characterization and phylogenetic analysis of Zika virus circulating in the Americas. Infect Genet Evol 43, 43-49, doi: 10.1016/j.meegid.2016.05.004 (2016).
34 Akiyama, B. M. et al. Zika virus produces noncoding RNAs using a multi-pseudoknot structure that confounds a cellular exonuclease. Science 354, 1148-1152, doi: 10.1126/science.aah3963 (2016).
35 Taglietti et al. Skin Ther. Lett. 13:6-8 (2008)
36 Shan, C. et al. A live-attenuated Zika virus vaccine candidate induces sterilizing immunity in mouse models. Nat Med, doi: 10.1038/nm.4322 (2017).
37 Xie, X. et al. Understanding Zika Virus Stability and Developing a Chimeric Vaccine through Functional Analysis. MBio 8, doi: 10.1128/mBio.02134-16 (2017).
38 Shan, C. et al. A Rapid Zika Diagnostic Assay to Measure Neutralizing Antibodies in Patients. EBioMedicine 17, 157-162, doi: 10.1016/j.ebiom.2017.03.006 (2017).
39 Lazear, H. M. et al. A Mouse Model of Zika Virus Pathogenesis. Cell Host Microbe 19, 720-730, doi: 10.1016/j.chom.2016.03.010 (2016).
40 Grant, A. et al. Zika Virus Targets Human STAT2 to Inhibit Type I Interferon Signaling. Cell Host Microbe 19, 882-890, doi: 10.1016/j.chom.2016.05.009 (2016).
41 Miner, J. J. et al. Zika Virus Infection during Pregnancy in Mice Causes Placental Damage and Fetal Demise. Cell 165, 1081-1091, doi: 10.1016/j.cell.2016.05.008 (2016)
42 Sapparapu, G. et al. Neutralizing human antibodies prevent Zika virus replication and fetal disease in mice. Nature 540, 443-447, doi: 10.1038/nature20564 (2016).
43 Richner, J. et al. Vaccine mediated protection against Zika virus induced congenital disease. Cell 170, 273-283 (2017).
44 Acceptability of cell substrates for production of biologicals. Report of a WHO Study Group on Biologicals. World Health Organ Tech Rep Ser 747, 1-29 (1987).
45 Organization, W. H. WHO/UNICEF Zika virus vaccine target product profile: Vaccine to protect congenital Zika syndrome for use during an emergence. (2017).
46 Pardi, N. et al. Zika virus protection by a single low-dose nucleoside-modified mRNA vaccination. Nature, doi: 10.1038/nature21428 (2017).
47 Ong, E. Z. et al. Preclinical evaluation of VIS513, a therapeutic antibody against dengue virus, in non-human primates. Antiviral Res 144, 44-47, doi: 10.1016/j.antiviral.2017.05.007 (2017).
48 Shan C, et al. A single-dose live-attenuated vaccine prevents Zika virus pregnancy transmission and testis damage. Nature Communications: in press (2017).

1. A live attenuated Zika virus (ZIKV) strain, comprising a deletion in the 3′ untranslated region (3′UTR) of the ZIKV genome.

2. The live attenuated ZIKV strain of claim 1, wherein the 3′UTR deletion ranges from a 10-nucleotide deletion to a 50-nucleotide deletion (i.e., Δ10, Δ11, Δ12, Δ13, Δ14, Δ15, Δ16, Δ17, Δ18, Δ19, Δ20, Δ21, Δ22, Δ23, Δ24, Δ25, Δ26, Δ27, Δ28, Δ29, Δ30, Δ31, Δ32, Δ33, Δ34, Δ35, Δ36, Δ37, Δ38, Δ39, Δ40, Δ41, Δ42, Δ43, Δ44, Δ45, Δ46, Δ47, Δ48, Δ49 or Δ50 3′UTR deletion).

3. The live attenuated ZIKV strain of claim 1, wherein the 3′UTR deletion is a 10-nucleotide deletion, a 20-nucleotide deletion, or a 30-nucleotide deletion.

4. The live attenuated ZIKV strain of claim 1, wherein the 3′UTR deletion is a 10-nucleotide deletion.

5. The live attenuated ZIKV strain of claim 1, wherein the 3′UTR deletion is a 20-nucleotide deletion.

6. The live attenuated ZIKV strain of any of the foregoing claims, comprising a 3′UTR having at least 80%, at least 85%, at least 90%, at least 95%, at least 98%, at least 99%, or 100% identity to the nucleic acid sequence of SEQ ID NO: 2, 3, 4, or 5.

7. (canceled)

8. (canceled)

9. (canceled)

10. (canceled)

11. The live attenuated ZIKV strain of claim 1, which is an mCherry ZIKV strain.

12. The live attenuated ZIKV strain of claim 1, which comprises or consists of a deletion variant of SEQ ID NO:6 wherein the sequence “CCAGAAGAGG” (3′UTR 10-nucleotide deletion) (SEQ ID NO:8) is deleted therefrom.

13. The live attenuated ZIKV strain of claim 1, which comprises or consists of a deletion variant of SEQ ID NO:6 wherein the sequence “CTGTGGATCTCCAGAAGAGG” (3′UTR 20-nucleotide deletion) (SEQ ID NO:9) is deleted therefrom.

14. The live attenuated ZIKV strain of claim 1, which comprises or consists of a deletion variant of SEQ ID NO:7 wherein the sequence “CCAGAAGAGG” (3′UTR 10-nucleotide deletion) (SEQ ID NO:8) is deleted therefrom.

15. The live attenuated ZIKV strain of claim 1, which comprises or consists of a deletion variant of SEQ ID NO:7 wherein the sequence “CTGTGGATCTCCAGAAGAGG” (3′UTR 20-nucleotide deletion) (SEQ ID NO:9) is deleted therefrom.

16. An immunogenic composition comprising a live attenuated ZIKV strain according to any of the foregoing claims, which further comprises at least one pharmaceutically acceptable carrier or excipient.

17. The immunogenic composition of claim 16, which is suitable for parenteral or enteral administration.

18. A method of eliciting an immune response in a subject in need thereof by administering a composition comprising a prophylactically or therapeutically effective amount of a live attenuated ZIKV strain according to claim 1.

19. (canceled)

20. (canceled)

21. The method of claim 18, wherein the subject is a pregnant female.

22. (canceled)

23. (canceled)

24. The method of claim 18, wherein at least 1.0×10¹, 1.0×10², 1.0×10³, 1.0×10⁴, 1.0×10⁵, or 1.0×10⁶ IFUs of the live attenuated ZIKV strain is administered to the subject.

25. (canceled)

26. (canceled)
